(19) United States
(12) Patent Application Publication
(10) Pub. No.: US 2015/0315651 A1
(43) Pub. Date: Nov. 5, 2015

(54) METHODS AND KITS FOR DETERMINING A PLACEBO PROFILE IN SUBJECTS FOR CLINICAL TRIALS AND FOR TREATMENT OF PATIENTS

(71) Applicant: Biometheus LLC, Naples, FL (US)

(72) Inventors: Gunther Winkler, Naples, FL (US); Kathryn T. Hall, Jamaica Plains, MA (US); Ted J. Kaptchuk, Cambridge, MA (US)

(21) Appl. No.: 14/801,820

(22) Filed: Jul. 16, 2015

Related U.S. Application Data
(63) Continuation-in-part of application No. 14/516,523, filed on Oct. 16, 2014.
(60) Provisional application No. 61/891,975, filed on Oct. 17, 2013.
(60) Provisional application No. 61/891,973, filed on Oct. 17, 2013.

(51) Int. Cl. C12Q 1/68

(52) U.S. Cl. CPC C12Q 1/6883; C12Q 2600/172; C12Q 2600/156; C12Q 2600/106

(57) ABSTRACT

The present invention is directed to methods and assays for identifying subjects participating in clinical trials that may exhibit a placebo response and identifying treatments for subjects with varying degrees of placebo responses. In one aspect, a method of selecting subjects to participate in a clinical trial is disclosed. In another aspect, methods for treating a subject and determining a treatment dosage are disclosed. In an exemplary embodiment, a method for determining a response to a treatment of a subject having, suspected of having, or at risk for developing a disorder, such as cardiovascular disorder, irritable bowel syndrome, diabetes, autoimmune disorders, inflammation, neurological disorders, chronic pain, cancer, cancer treatments, allergies, depression, migraines, addiction, obesity, and other disorders, syndromes, or diseases, is disclosed.

### RELATED APPLICATIONS

This patent application is a continuation-in-part of, and claims the benefit of, U.S. patent application Ser. No. 14/516,523 filed Oct. 16, 2014 entitled METHODS AND KITS FOR DETERMINING A PLACEBO PROFILE IN SUBJECTS FOR CLINICAL TRIALS AND FOR TREATMENT OF PATIENTS, and naming Gunther Winkler, Kathryn T. Hall, and Ted J. Kaptchuk as inventors, which claims the benefit of U.S. Provisional Patent Application No. 61/891,973 filed on Oct. 17, 2013, entitled METHODS AND KITS FOR DETERMINING A PLACEBO PROFILE IN SUBJECTS FOR CLINICAL TRIALS, naming Gunther Winkler, Kathryn T. Hall, and Ted J. Kaptchuk as inventors and U.S. Provisional Patent Application No. 61/891,975 filed on Oct. 17, 2013, entitled METHODS AND KITS FOR DETERMING A PLACEBO PROFILE IN SUBJECTS FOR CLINICAL TRIALS, naming Gunther Winkler, Kathryn T. Hall, and Ted J. Kaptchuk. The entire content of the foregoing applications are incorporated herein by reference, including all text, tables and drawings.

### SEQUENCE LISTING

The instant application contains a Sequence Listing which has been submitted electronically in ASCII format and is hereby incorporated by reference in its entirety. Said ASCII copy, created on Dec. 29, 2014, is named Biometheus0434399_ST25.txt and is 16,480 bytes in size.

### FIELD OF THE INVENTION

Technology provided herein relates, in part, to human genotypes associated with placebo responses and human genotypes associated with differential responses to medical treatments, methods of conducting controlled treatment studies to account for such genotypes in a population, and method of treating subjects or altering treatment of subjects having specific genotypes identified herein.

### BACKGROUND OF THE INVENTION

Placebo effects are ubiquitous in clinical care and present both opportunities and significant issues. Opportunities exist for caregivers to enhance therapeutic effects of medications and medical procedures through exploitation of a patient's capability of a healing placebo response. It has been demonstrated herein that clinical intervention paired with placebo responses can significantly improve outcomes for patients.

In addition, placebo effects significantly impact the success of the development of new therapeutic interventions. Increasingly, many drugs, medical devices and clinical procedures that have considerable evidence and early demonstration of efficacy are not further developed because of the prohibitive cost of demonstrating the FDA required superiority to placebo controls in large clinical trials. This is one critical factor that has led to a slowing time to registration and has led to sharply rising costs of medicines and medical devices. Methods for reducing the size and cost of the required clinical trials and still demonstrating superiority to placebo is desired and has been sought after by the US government and especially by the FDA which has established a “Critical Path Initiative” to explore faster and cheaper development of new drugs, devices and procedures. Methods to reduce and control placebo effects during clinical trials are a critical element in achieving this goal. It has been known that some patients have a natural propensity to increased placebo response and it is desirable to identify and to exclude such patients from clinical trials. However, until now, methods to identify such patients prior to enrollment into clinical trials have failed or have provided inconsistent and unpredictable results. Establishing a systematic and accurate way to identify placebo responders and thereby predicting or even controlling placebo effects in clinical trials would be a major step towards more efficient, faster and cheaper drug development which would greatly benefit health care developers, patients and society in general.

### SUMMARY OF THE INVENTION

Presented herein, in some embodiments, is a method of conducting a randomized clinical trial comprising a) detecting a genotype of a placebo-associated polymorphism in a sub-population of human subjects, wherein the human subjects are candidates for a clinical trial, thereby providing a first sub-group of subjects comprising a first genotype of the polymorphism and a second sub-group of subjects comprising a second genotype of the polymorphism, wherein the first and second genotypes are not the same, b) distributing the first sub-group evenly, unevenly and/or randomly into at least a first study group and a second study group, wherein at least the first study group is administered a first treatment and the second study group is administered a placebo treatment. In some aspects after administering the first treatment and the placebo treatment, a response is measured from the subjects of the first and second study groups, and wherein the safety and/or efficacy of the first treatment is evaluated by comparing the measured response between one or more subjects of the first and second study groups. In some embodiments the second subgroup is excluded from at least the first and second study groups. In some embodiments the second subgroup is excluded from participating in the randomized clinical trial. In some embodiments the second genotype is associated with an enhanced placebo response. In certain embodiments the second subgroup is distributed evenly, unevenly and/or randomly into at least the first study group and the second study group. In certain embodiments the method comprises providing a third sub-group of subjects comprising a third genotype of the polymorphism, wherein the third genotype is different than the first and the second genotype. In some aspects the third sub-group is associated with an enhanced placebo response and wherein the third sub-group is excluded from at least the first and second study groups. In some aspects the third sub-group is distributed evenly, unevenly and/or randomly into at least the first study group and the second study group. The first genotype can comprise a homozygous variant of the polymorphism or a heterozygous variant of the polymorphism. The second genotype can comprise a homozygous variant of the polymorphism or a heterozygous variant of the polymorphism. In some embodiments the placebo-associated polymorphism is selected from a placebo-associated polymorphisms in Table 5. In some embodiments the placebo-associated polymorphism is a COMT polymorphism. In some embodiments the COMT polymorphism is selected from the group consisting of rs4680, rs4818, rs6269, rs4633, rs4485648 and rs740601. In some embodiments the placebo-associated polymorphism is selected from the group consisting of rs6323, rs6609257, rs2873804, rs6280, rs6265, rs4570625, rs4251417, rs2296972, rs622337, rs510769, rs324420, rs1611115, and rs1799971. In some embodiments the first experimental treatment comprises administering a pharmaceutical composition for the treatment of a disorder or a condition selected from the group consisting of irritable bowel syndrome, diabetes, an autoimmune disorder, inflammation, a neurological disorder, chronic or acute pain, cancer, allergies, depression, migraines, addiction, obesity and cardiovascular disease. In some embodiments the response is a symptom or clinical characteristic of the disorder or condition. In some embodiments the first experimental treatment comprises administering an analgesic, a drug that interacts with an opioid pathway, a drug that interacts with a serotonin pathway, a drug that binds directly to a COMT protein, a drug that inhibits an activity or function of a COMT protein, a beta-blocker, an alpha-blocker, an agonist of alpha adrenergic receptor, or a agonist of beta-adrenergic receptor blocker. In certain embodiments the second genotype comprises a placebo allele listed in Table 6. In some embodiments the genotype is selected from an A/A homozygote, A/G heterozygote and G/G homozygote of an rs4680 COMT polymorphism.

In some embodiments presented herein is a method of treating a subject having a placebo-associated polymorphism with a placebo comprising, a) administering a treatment to a human subject having, or are at risk of having a disorder or condition, and wherein the treatment is indicated for the disorder or condition, b) determining an efficacy of the treatment according to a response of the human subject to the treatment, wherein the response is a clinical characteristic or symptom of the disease or disorder, c) determining a genotype for a placebo-associated polymorphism in the subject and d) administering the treatment and a placebo treatment to the subject if the genotype is associated with an enhanced placebo effect. In some embodiments after d), the method comprises repeating the determining step of b), and if the efficacy determined is substantially greater than the efficacy determined in b), then either i) continue administering the treatment and placebo treatment to the subject as needed or ii) continue administering the placebo treatment and administer a lower dosage of the treatment.

In some embodiments presented herein is a method of treating a subject having a placebo-associated polymorphism with a placebo comprising, a) administering a treatment to a human subject having, or are at risk of having a disorder or condition, and wherein the treatment is indicated for the disorder or condition, b) determining an efficacy, and a presence or degree of an adverse effect of the treatment according to one or more responses of the human subject to the treatment, c) determining a genotype for a placebo-associated polymorphism in the subject and d) if the genotype is associated with an enhanced placebo effect, administering the treatment and a placebo treatment to the subject, wherein the amount or dosage of the treatment is reduced compared to the amount administered in a). In some embodiments after d), the method comprises repeating the determining step of b), and if i) the efficacy determined is the same or greater than the efficacy determined in b), and ii) the presence or degree of the adverse effect is eliminated or reduced compared to the presence or degree of the adverse effect determined in b), then continue administering the reduced treatment and placebo treatment to the subject as needed.

In some embodiments presented herein is a method of identifying a genotype of a placebo associated polymorphism that is associated with an enhanced placebo response comprising a) detecting two or more genotypes of a placebo-associated polymorphism in a sub-population of human subjects, wherein the human subjects have or are suspected of having a disorder or condition, thereby providing a first sub-group and second sub-group, the first sub-group of subjects comprising a first genotype of the polymorphism and a second sub-group of subjects comprising a second genotype of the polymorphism, wherein the first and second genotypes are not the same, b) administering a substantially same placebo treatment to one or more subjects of the first and second sub-groups, c) measuring a response of one or more subjects in the first and second sub-groups, wherein the response is a clinical characteristic or symptom of the disorder or condition, and d) comparing the response to the placebo treatment between one or more subjects of the first and second sub-groups, wherein an improvement in the response of one or more subjects in the first sub-group compared to the second sub-group indicates the genotype of the placebo associated polymorphism of the first sub-group is associated with an enhanced placebo response. An improvement in the response is sometimes a reduction or elimination of one or more adverse symptoms.

Also presented herein is a method of identifying a genotype of a placebo associated polymorphism that is associated with an enhanced treatment response comprising a) detecting two or more genotypes of a placebo-associated polymorphism in a sub-population of human subjects, wherein the human subjects have or are suspected of having a disorder or condition that is treatable by a first treatment, thereby providing a first sub-group and second sub-group, the first sub-group of subjects comprising a first genotype of the polymorphism and a second sub-group of subjects comprising a second genotype of the polymorphism, wherein the first and second genotypes are not the same, b) administering a substantially same treatment to one or more subjects of the first and second sub-groups, c) measuring a response of one or more subjects in the first and second sub-groups, wherein the efficacy and/or safety of the treatment is determined by measuring the response, and d) comparing the efficacy and/or safety of the treatment between one or more subjects of the first and second sub-groups, wherein an increase in the efficacy and/or safety of one or more subjects in the first sub-group compared to the second sub-group indicates the genotype of the placebo associated polymorphism of the first sub-group is associated with an enhanced treatment response to the first treatment. In some embodiments the subjects of the first and second sub-groups are distributed evenly, unevenly or randomly among two or more study groups. In some embodiments the response of each subject of the first and second sub-groups is individually tracked and/or monitored.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1A is a bar graph showing the effect of the rs4680 COMT genotype on change in IBS-SSS after receiving a placebo treatment. Number of Val158met met alleles showed a significant linear effect on IBS-SSS (beta=0.17; p=0.032). IBS-SSS includes abdominal pain severity, abdominal pain frequency, abdominal distention severity, dissatisfaction with bowel habits, and disruption of quality of life. Change in IBSSSS=(IBS-SSS at baseline−IBS-SSS at 3-weeks). Regression model included COMT genotype (number of met alleles) and baseline IBS-SSS. Error bars indicate the standard error of the mean. N=104.

FIG. 1B is a bar graph showing the effect of the rs4633 COMT genotype on change in IBS-SSS after receiving a placebo treatment.

FIG. 1C is a bar graph showing the effect of the rs4818 COMT genotype on change in IBS-SSS after receiving a placebo treatment.

FIG. 2A is a bar graph showing the interaction effect of rs4680 COMT genotype and placebo treatment arms on change in IBS-SSS. The interaction between COMT genotype (number of met alleles) and treatment arm was statistically significant (beta=0.17; p=0.035). Regression model included the following parameters: COMT genotype (number of met alleles), treatment arm, baseline IBS-SSS and their interaction (COMT genotype x treatment arm). Error bars indicate the standard error of the mean. N=104.

FIG. 2B is a bar graph showing the interaction effect of rs4633 COMT genotype and placebo treatment arms on change in IBS-SSS.

FIG. 2C is a bar graph showing the interaction effect of rs4818 COMT genotype and placebo treatment arms on change in IBS-SSS.

FIG. 3A is a bar graph showing the interaction effect of rs4680 COMT genotype and placebo treatment arms on Adequate Relief. The interaction (COMT genotype x treatment arm) was statistically significant (beta=0.25; p=0.009), but not COMT genotype (beta=0.01; p=0.954). Adequate Relief was assessed by a single dichotomous categorization at three weeks, which asked patients: “Over the past week have you had adequate relief of your IBS symptoms?” Regression model parameters included: COMT genotype (number of met alleles), treatment arm and their interaction (COMT genotype×treatment arm). Error bars are standard error of the mean. N=102.

FIG. 3B is a bar graph showing the interaction effect of rs4633 COMT genotype and placebo treatment arms on Adequate Relief.

FIG. 3C is a bar graph showing the interaction effect of rs4818 COMT genotype and placebo treatment arms on Adequate Relief.

FIG. 4 shows results of a placebo study testing the placebo response of subjects to a pain stimulus. Each bar represents a quantitation of placebo effect (y-axis) of subjects comprising a Val/Val genotype (Val/Val), Val/Met genotype (Val/Met) or a Met/Met genotype (Met/Met) for amino acid 158 of COMT (COMT Val158met). N=48. (p=0.058) The study shown was conducted at Mass General Hospital, Harvard Medical School.

### DETAILED DESCRIPTION OF THE INVENTION

As described herein, certain genotypes of a polymorphism can differentially effect a subjects response to a treatment (e.g., a medical treatment) or to a placebo.

A polymorphism is a natural variation of a DNA sequence occurring at a specific location in the genome of a species (e.g., humans). A polymorphism is often present in a population in two or more possible variations (e.g., as two or more specific sequence variants). A polymorphism often exist within a chromosome of in one of the two or more variations. In a diploid individual having two copies of each chromosome, a polymorphism can exist in one form or another on each chromosome, thereby defining two alleles. For example, for hypothetical polymorphism B having two possible variants (e.g., adenine (A) or guanine (G)) at position Z of chromosome 21, an individual may have an A at position Z on one copy of chromosome 21 defining a first allele and a G at position Z on the other copy of chromosome 21 defining a second allele. Collectively, the genetic make up of a specific variant of a polylmorphism present on both alleles in a diploid individual is referred to herein as a genotype of the polymorphism. A genotype of a polymorphism can be homozygous for a specific sequence variant

Sometimes a polymorphism, in one form or another, has no detectable or known effects on an individual. However, some polymorphism, in one form or another, due to having an effect on a gene or gene product, can result in different phenotypes within a population. As described herein, subjects having specific genotypes respond differently to certain medical treatments and/or to placebo treatment. In certain embodiments, with such knowledge in hand, clinical trials are designed to remove subjects with specific genotypes/phenotypes (e.g., subjects having a genotype associated with a strong placebo response) from an experimental trial. In some embodiments, clinical trials are designed to evenly and/or randomly distribute subjects with specific genotypes/phenotypes among study groups for the purpose of conducting a clinical trial. This process is sometimes referred to as blocking. In certain embodiments, blocking comprises dividing subjects into subgroups called blocks according to common genotypes. Blocks can then be distributed in the same or different study groups where each study group is administered a treatment. The response (e.g., a response to a treatment) of subjects within a block can be tracked independently, controlled for and/or combined with other blocks for statistical analysis. Such a process is often known as blocked randomization. Therefore, in certain embodiments, subjects (e.g., blocks or subgroups of subjects) can be tracked and their response to a treatment can be individually monitored, assessed and compared to other genotypes within a study. In some embodiments, subjects having a specific genotype identified herein respond differently to a medical treatment and such subjects and their corresponding genotype can be identified by tracking and/or blocking. In some embodiments, subjects having a specific genotype identified herein respond differently to a medical treatment and such subjects, when tracked or blocked, and/or any data derived from such subjects can be excluded from a study (e.g., from a trial, from an analysis). In some embodiments, subjects having a specific genotype identified herein respond differently to a medical treatment, and therefore treatments can be modified for such subjects according to their genotype in an effort to optimize the beneficial effects of a treatment.

Polymorphisms have been identified herein that, in one variant form or another, affect a subjects response to a treatment and/or to a placebo treatment. These polymorphisms are collectively referred to as placebo-associated polymorphisms (PAPs). In some embodiments, a placebo-associated polymorphism (PAP) is one selected from Table 5. In some embodiments, PAP is a COMT polymorphism (e.g., rs4680 (Val158met)). As described herein, the modulation of a placebo response or effect conferred by one or more PAPs may have implications for personalized medicine, treatment regimens and development of strategies in conducting clinical trials.

Certain exemplary embodiments will now be described to provide an overall understanding of the principles of the structure, function, manufacture, and use of the compositions and methods disclosed herein. One or more examples of these embodiments are illustrated in the accompanying drawings. Those skilled in the art will understand that the compositions and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Subjects

A subject can be any living or non-living organism, including but not limited to a human, non-human animal, plant, bacterium, fungus, virus or protist. A subject is sometimes a human subject. A subject may be any age (e.g., an embryo, a fetus, infant, child, adult). A subject can be of any sex (e.g., male, female, or combination thereof). A subject may be pregnant. A subject can be a patient (e.g. a human patient).

In some embodiments a subject has or is suspected of having a disorder, ailment, disease or condition. In some embodiments a condition includes, for example, damage or injury (past or present), psychological conditions, genetic conditions, chronic or acute conditions, and the like or combinations thereof. A condition can be acute or chronic. A disorder can be a genetic disorder (e.g., a disorder, ailment or disease associated with a genetic variation in a subjects genome). A subject can be a subject with a predisposition (e.g., a genetic predisposition) towards developing a disorder. A subject may be suspected of having a disorder due to a genetic predisposition of having said disorder. In some embodiments, a subject comprises a genetic variation and is determined to have a disorder due to the presence of the genetic variation.

Subjects further include those that are at increased risk of developing a disorder or disease. “At risk” subjects typically can have risk factors for developing or having a particular disorder or disease. Non-limiting examples of risk factors include a family history (e.g., genetic predisposition), the presence or absence of a genetic variation, gender, race, age, lifestyle (e.g., diet, smoking), occupation, environmental factors (e.g., exposure), the like or combinations thereof.

In some embodiments a subject is a subject in need of a treatment (e.g., a medical treatment, an experimental treatment) or drug (e.g., pharmaceutical drug, experimental drug). In some embodiments a subject in need of a treatment or drug is a subject having or suspected of having a disorder, disease or condition. In some embodiments a subject in need of a treatment or drug is a subject experiencing one or more symptoms of a disorder, disease or condition. In some embodiments a subject in need of a treatment or drug is a subject at risk of developing a disorder or disease. In some embodiments a subject in need of a treatment or drug is, or is suspected of being, infected with a pathogen.

In certain embodiments a subject having, suspected of having or at risk of having a disorder, ailment, disease or condition is considered for participating in a clinical trial or study group. In certain embodiments a subject having, suspected of having or at risk of having a disorder, ailment, disease or condition is a candidate for participating in a clinical trial or is a candidate for receiving a treatment. A candidate for participating in a study (e.g., a clinical trial) is often a subject who has, or is at risk of having a disorder, ailment, disease or condition, where at least one treatment administered during the study is being evaluating for the treatment of said disorder, ailment, disease or condition. A treatment is often evaluated by determining a response of a subject to the treatment. In certain embodiments a subject having, suspected of having or at risk of having a disorder, ailment, disease or condition is selected to participate in a clinical trial by a method described herein. In certain embodiments a subject having, suspected of having or at risk of having a disorder, ailment, disease or condition is selected to receive a treatment by a method described herein. In certain embodiments a subject having, suspected of having or at risk of having a disorder, ailment, disease or condition is selected to receive a placebo treatment by a method described herein.

Symptoms and Responses

In some embodiments a response is determined or measured. A response can often be a measured response. A response can be subjective or objective. In some embodiments a measured response is a recorded response. A measured response may be directly measured or indirectly measured. In certain embodiments a response is the presence of, absence of, change in, or amount of a suitable physiological parameter. Non-limiting examples of a physiological parameter include symptoms (e.g., objective symptoms and subjective symptoms), blood pressure, body temperature, heart rate, heart volume, any suitable blood or serum agent or substance (non-limiting examples of which include a metabolite, urea, creatinine, a sugar or metabolite thereof (e.g., glucose, lactose, lactic acid, etc.), cells (e.g., red blood cells, lymphocytes, macrophages, eosinophils, platelets, etc.), a nucleic acid, salts (e.g., sodium, potassium, etc.), proteins or protein markers (e.g., cytokines, chemokines, hormones, enzymes, receptors, ligands, etc.), lipids starches, drugs or drug metabolites thereof, the like or combinations thereof), bone density, images of the body or a parts thereof (e.g., X-rays, ultrasound, CAT scan, etc.), mobility or lack thereof, physical tasks (e.g., walking, running, jumping, sitting, and the like), range of motion of a body part, sensations (e.g., of pain, heat, cold, itching, prickliness, touch, pressure, the like, the lack thereof, or combinations thereof), cognitive abilities, vision, hearing, taste, smell, the like, the lack thereof, or combinations thereof. A physiological parameter can be observed, reported by a subject and/or measured. Certain physical parameters are sometimes one or more clinical characteristics that are associated with the presence of a disorder, condition or disease. In some embodiments a measured response comprises determining the presence, absence or degree of a symptom. For example, measuring a response can comprise determining an increase, decrease (e.g., reduction) or no change of a symptom.

In some embodiments a subject is experiencing one or more symptoms. Symptoms can be chronic or acute. A symptom can be constant or intermittent. In certain embodiments a symptom can be the absence of a normal sensation or biological process. A symptom can be caused by any disorder, disease or condition. In some embodiments a cause of a symptom is not known. In some embodiments a symptom is related to a psychological disorder. In some embodiments a symptom is manifested by a psychological disorder. In some embodiments a symptom is a neurological symptom or related to a neurological disorder, disease or condition.

In certain embodiments a symptom can be any measureable and/or observable symptom (e.g., a symptom that can be measured and/or observed by a medical professional). Non-limiting examples of measurable and/or observable symptoms include inflammation, swelling, infection, organ failure, hyperplasia, the presence of cancer (e.g., a tumor, lymphoma), coughing, wheezing, temperature (e.g., fever, hypothermia, hyperthermia), the presence of a pathogen, rash, weight gain, jaundice, bruising, convulsions, seizures, discharge, bleeding, sweating, tremors, urinary incontinence, bowel obstruction, arrhythmia, depression, psychosis, pain, the like or combinations thereof.

In certain embodiments a symptom is a subjective symptom. A subjective symptom can be a subject's perception of any physical or mental symptom. In some embodiments a subjective symptom is a symptom experienced by a subject where the presence, absence, description and/or degree (e.g., amount, intensity) of the symptom is determined according to a subjective response from the subject experiencing the symptom. In some embodiments the presence, absence, description and/or degree of a subjective symptom can only be assessed by a subjective response. A subjective response can include any suitable audible and/or physical response that communicates the presence, absence, description and/or degree of a symptom to a another (e.g., a third party, a medical professional). Non-limiting examples of audible responses include one or more sounds, gestures, verbal responses and/or spoken words. Non-limiting examples of a physical response include gestures, signs (e.g., use of sign language), physical movements (e.g., any suitable movement of a body part, twitching, reflex movements, the like, or combinations thereof), drawing, writing (e.g., written words, written responses), the like or combinations thereof. A response can be communicated directly or indirectly to another. For example, a response can be communicated indirectly to another by means of a communication device (e.g., a computer, cell phone, an app (e.g., a health tracking app)) and the like).

A subjective response is often a response to a suitable sensory stimuli configured to induce a response. In some embodiments a sensory stimuli is a query or question directed to a subject. Often a sensory stimuli is a question asking a subject about the presence, absence or degree of a symptom. In some embodiments a sensory stimuli is a question asking a subject to describe a symptom. In certain embodiments a sensory stimuli is a question asking a subject about the presence, absence or degree of a stimuli. In certain embodiments a sensory stimuli is a question asking a subject about the presence, absence or degree of a symptom during or following a treatment. In some embodiments a subjective response is a subject's answer to a question regarding a symptom (e.g., regarding the presence, absence, description and/or degree of a symptom). In some embodiments a sensory stimuli is a visual stimuli. Non-limiting examples of visual stimuli include written questions, pictures, light, color, shades, shapes, the like, changes thereof, or combinations thereof. In some embodiments a sensory stimuli is a sound (e.g., music), smell, and/or taste (a food or drink). In some embodiments a sensory stimuli is an epidermal stimuli, non-limiting examples of which include a suitable touch (e.g., grasp, hold, poke, prick, brush) wind, heat, cold, electrical stimuli, the like or combinations thereof. In some embodiments a subjective response is not in response to a stimuli configured to induce a response. For example, sometimes a subject provides a voluntary subjective response regarding a symptom without a stimuli or provocation.

Non-limiting examples of a subjective symptom include any pain, (e.g., phantom pain, headache, muscle pain, aches, abdominal (stomach) pain, chest pain, heart burn, and the like), discomfort, pressure, cramping, sensations of constipation, sensations of bloating, nausea, feeling sick, tingling, sensations of touch, sensations of warmth, heat, hot flashes, burning, cold, freezing, weakness, fatigue, anxiety, restlessness, restless leg syndrome, vestibular sensations (e.g., dizziness, balance, spatial orientation), inability to concentrate, memory problems, sensations of sight/vision, blurred vision or blindness (e.g., without a biological, biochemical or physiological explanation), anger, cravings, disrupted mood, optical flashes or pulses of light, perception of color, light, and/or darkness, depression, psychosis, hallucinations, insomnia, hearing and auditory sensations, tinnitus, sensations of smell, the like, presence thereof, lack thereof, or combinations thereof.

A differential response refers to two or more subjects that have a different response to the same treatment. A differential response may correlate with different genotypes. For example, a first group of subjects who are homozygous for a variant of a polymorphism may have a significantly different response to a treatment than a second group of subjects that are homozygous for a second variant of the polymorphism. Subjects having different genotypes of a PAP may have similar or different phenotypes and may have similar or differential responses to the same treatment. In some embodiments a differential response refers to a statistically significant difference in a measured response between two or more groups of subjects. A placebo-associated polymorphism is sometimes associated with a differential response of two groups of subjects to a treatment where the two groups of subjects differ in genotype for the polymorphism.

PAPs

In some embodiments a placebo-associated polymorphism is a polymorphism that is associated with a differential response to a treatment where subjects having a first genotype representing at least one variant of the polymorphism respond differently to a treatment when compared to subjects having a second genotype representing a different variant of the polymorphism. In some embodiments, a PAP comprises a single nucleotide polymorphism (SNP). In some embodiments a placebo-associated polymorphism is a polymorphism that is associated with a differential response to a treatment between subjects having a first genotype representing the presence of the polymorphism and subjects having a second genotype representing an absence of the polymorphism. In some embodiments a placebo-associated polymorphism is a polymorphism that is associated with a differential response to a treatment between subjects having a first genotype representing a homozygote of a specific variant of a polymorphism (e.g., a specific variant present on both alleles) and subjects having a second genotype representing the heterozygote of the same polymorphism (e.g., a specific variant present on only one allele). Table 5 and Table 6 in Example 5 provides a list of PAPs.

A genotype of a human subject can comprise the presence and/or absence of one or more PAPs. In some embodiments a subject comprises the presence or absence of 1 to 200, 1 to 100, 1 to 50, 1 to 20, 1 to 10, or 1 to 5 PAPs. In some embodiments a subject comprises the presence or absence of 1, 2, 3, 4, 5, 6, 7, 8, 9 or 10 PAPs, some or all of which can be associated with a placebo effect and/or an increased likelihood that a subject will have a placebo response.

COMT

Catecholamines play a key role in cognitive, behavioral, sensory, endocrine and autonomic nervous system regulation. Thus functional polymorphisms in catechol-O-methyltransferase (COMT, EC2.1.1.6, UniProtKB: P21964), an enzyme that metabolizes catecholamines is associated with a variety of clinical conditions. Catecho-O-methyltransferase (COMT) is an enzyme that catalyzes the O-methylation of various compounds, like catechol estrogens and dietary polyphenols, using S-adenosylmethionine (SAM) as the methyl donor and has also a role in dopamine inactivation. Dopamine is a catecholamine neurotransmitter or hormone that is important in signaling in the reward-motivation and motor control neural pathways. Dysfunction in the dopamine system is associated with several diseases and disorders including schizophrenia, attention deficit hyperactivity disorder, addiction and Parkinson's for example.

COMT is encoded on human chromosome 22 (Entrez Gene ID: 1312, NCBI Reference Sequence: NG 011526.1 showing nucleotides 1 to 35236 of human COMT gene) and several polymorphisms in the gene can affect the expression and function of the enzyme. As described herein, the presence of a polymorphism (e.g., a particular genotype of a polymorphism) in a COMT gene can be associated with a placebo response and/or an increased likelihood that a subject will have a placebo response. One or more PAPs can be in a COMT gene, a transcribed region of a COMT gene, a COMT intron, a COMT exon, a COMT coding sequence, a regulatory regions that can effect COMT expression (e.g., mRNA expression or protein expression), the like or combinations thereof. In some embodiments the presence of a particular polymorphism in a COMT nucleic acid results in an amino acid mutation (e.g., an amino acid substitution, insertion or deletion). In some embodiments an amino acid mutation in a COMT polypeptide reduces COMT enzyme activity compared to a wild-type (e.g., non-mutated enzyme) by 2-fold or more, 3-fold or more, 4-fold or more, 5-fold or more or 6-fold or more. In certain embodiments reduced COMT activity is associated with a placebo effect and/or an increased likelihood that a subject will have a placebo effect. In some embodiments the presence of a PAP in a COMT gene (e.g., a particular genotype of a COMT polymorphism) results in the reduced expression of a COMT polypeptide. In some embodiments the presence of a COMT polymorphism reduces expression of a COMT polypeptide compared to expression of a COMT polypeptide from a wild-type gene (e.g., a majority genotype, the absence of a COMT polymorphism, a COMT gene lacking said polymorphism) by 2-fold or more, 3-fold or more, 4-fold or more, 5-fold or more or 10-fold or more. In certain embodiments the presence of a polymorphism in a COMT nucleic acid that reduces expression of a COMT polypeptide is associated with a placebo effect and/or an increased likelihood that a subject will have a placebo effect. In some embodiments a method comprises selecting a sub-group of human subjects wherein the presence of a COMT polymorphism is detected in subjects of the sub-group and the presence of the COMT polymorphism results in reduced activity or reduced expression of a COMT polypeptide.

The presence of a polymorphism (e.g., a PAP) refers to the presence of a specific allele of a polymorphism. The presence of a specific allele can refer to a heterozygous or homozygous genotype comprising the allele. A detected polymorphism in a COMT gene can be present in one or both alleles (e.g., both alleles of a genotype for a diploid subject). In some embodiments a detected COMT polymorphism is present in one allele and the subject is heterozygous for the COMT polymorphism (e.g., heterozygous for the presence of the COMT polymorphism). In certain embodiments a detected COMT polymorphism is present in both alleles and the subject is homozygous for the COMT polymorphism (e.g., homozygous for the presence of the COMT polymorphism). In some embodiments the presence of a COMT polymorphism refers to the presence of a recessive allele of a polymorphism. In some embodiments the presence of a COMT polymorphism refers to the presence of a minor allele of a polymorphism. In some embodiments the presence of a COMT polymorphism indicates the presence of a specific genotype in a subject. A genotype can include one or more than one COMT polymorphisms. For example the presence of a COMT polymorphism can refer to a genotype that includes the presence of an rs4680 polymorphism and the presence of an rs4618 polymorphism.

The COMT Val158met or rs4680 polymorphism is a G to A transition at codon 158 (e.g., amino acid 158, P21964) in the transmembrane form and codon 108 (e.g., amino acid 108) in the secreted form of the COMT protein. The COMT rs4680 polymorphism occurs at nucleotide number 973 of the COMT mRNA transcript variant X1 shown herein. The rs numbers (“rs#”; “refSNP cluster ID number”) shown herein represent a well-known nomenclature for single nucleotide polymorphisms (SNP). The reference rs numbers and information regarding their representative SNPs (e.g., nucleotide positions) can be searched and found online at URL http://www.ncbi.nlm.nih.gov/SNP/, accessed Oct. 13, 2014, for example. The G allele of rs4680 codes for a valine and is sometimes referred to herein as the Val158 allele. The A allele of rs4680 codes for methionine and is sometimes referred to herein as the Met158 allele. The presence of an rs4680 polymorphism refers to a genotype of a subject having at least one A allele of the rs4680 polymorphism. The presence of an A allele can refer to a heterozygous or homozygous genotype comprising an A allele of an rs4680 polymorphism. In some embodiments, detection of the presence of the A allele of rs4680 (Met158) determines the presence of an rs4680 polymorphism. In some embodiments, detection of the absence of an rs4680 refers to detecting the absence of an A allele of an rs4680 polymorphism. In some embodiments, detection of the presence of two G alleles of an rs4680 (Val158)(e.g., a G/G homozygote) determines the absence of an rs4680 polymorphism. The presence or absence of an rs4680 polymorphism can be determined by a suitable method or by a method described herein. The Val158 form of a COMT polypeptide is 3 to 4-fold more active than the Met158 form at body temperature. As a result of this mutation, homozygotes for the Val158 allele, in the absence of substances that block the enzyme, have been shown to have significantly lower levels of catecholamines e.g. dopamine and epinephrine as compared to Met158 allele homozygotes. This functional polymorphism has been correlated with variations in memory function, cognition, attentional processing, high blood pressure, pain processing and sensitivity.

Other polymorphisms in COMT including rs4818 and rs4633 have been shown to affect the stability of the COMT messenger RNA and thus the expression levels of the enzyme. The rs4633 polymorphism represents a C to T transversion. The presence of an rs4633 polymorphism refers to a genotype of a subject having at least one T allele of the rs4633 polymorphism. The presence of an rs4818 polymorphism refers to a genotype of a subject having at least one C allele of an rs4818 polymorphism. The rs6269 polymorphism represents a A to G transversion. The presence of an rs6269 polymorphism refers to a genotype of a subject having at least one G allele of the rs6269 polymorphism.

Detecting the presence of a COMT polymorphism can include detecting the presence of one or more COMT polymorphisms. In certain embodiments detecting the presence of a COMT polymorphism comprises detecting the presence and/or absence of an rs4680 polymorphism, an rs4818 polymorphism, an rs6269 polymorphism, and an rs4633 polymorphism, alone or in combination. For example, a subjects genotype can comprise the presence of an rs4680 polymorphism, the presence of an rs4818 polymorphism and the absence of an rs4633 polymorphism.

The data presented in the Examples section herein suggests that genetic variations that change the expression or activity of COMT can affect a subject's response to a placebo or treatment. For example, a significant correlation of the rs4680 COMT polymorphism and the resulting response to a placebo treatment or a placebo inducing therapeutic encounter between a subject and a caregiver is demonstrated herein. In some embodiments, a therapeutic encounter with a caregiver is considered an active component of a non-specific placebo treatment. For example in some embodiments the presence of a therapeutic encounter is used to identify reliable predictors of a placebo effect. Other studies did not include an arm in which a placebo treatment was administered within the context of an augmented therapeutic encounter. Indeed, many of these studies were done with volunteer subjects in experimental rather than clinical contexts in which there was no therapeutic encounter at all.

Some skilled in the art believe it is unlikely that a single gene locus (e.g., like a polymorphism in COMT) can fully account for a complex behavioral phenotype such as a placebo response. The data presented suggests that a single nucleotide polymorphism in COMT (e.g., like rs4680) can account for a significant increase in a subjects placebo response. Therefore, the data presented herein was surprising and unexpected.

In some embodiments, a COMT polymorphism is at least an rs4680, rs4818, rs6269, and/or rs4633 polymorphism. A COMT polymorphism can be an rs4680 polymorphism and encode a methionine/methionine haplotype, a valine/methionine haplotype, or a valine/valine haplotype. Some examples of COMT polymorphisms, but not limited to the listed COMT polymorphisms, that can be used with the methods disclosed herein are show in Table 1.

TABLE 1
COMT (SEQ. ID. NOs: 1-75) Polymorphisms

| COMT | Context Sequence |
|---|---|
| rs1055503 | AAGGGAGGGGAGGGGGCCCCGACGG[A/G]GCCACAGTAAAGGAGTGGAGG |
| | GGCC |
| rs1109436 | TGCAATTCAAAATCAAGGGCTGCTT[C/T]GAGGAGGCCTCTCCACCGGGCTG |
| | CT |
| rs11544670 | TGCCGGAGGCCCCGCCTCTGCTGTT[G/T]GCAGCTGTGTTGCTGGGCCTGG |
| | TGC |
| rs11569716 | TCCGTGTCTGCTTCTGTATTTTGTG[C/T]GGTTTTAGAGGATCCCTGGGCTGCC |
| rs12814 | CCCGGCCCCCCTCTCGGGCTCTCTC[A/T]CCCAGCCTGGTACTGAAGGTGCC |
| | AG |
| rs13055002 | TGGCCCCAGGGGCTAGGCACAGGCG[C/T]GGTGCCGTGGCCTAGTGAGGA |
| | GCAC |
| rs13306281 | CGAGCAGAAGGAGTGGGCCATGAAC[A/G]TGGGCGACAAGAAAGGTGGGGT |
| | CCG |
| rs1544325 | GAGACAAATTAGAAATGTCAGTCTG[A/G]AGAGAGTGGTAGGTAGCCAGATA |
| | CT |
| rs165599 | TGTTAGCCCCATGGGGACGACTGCC[A/G]GCCTGGGAAACGAAGAGGAGTC |
| | AGC |
| rs165631 | ACCGGTACCTGCCGGACACGCTTCT[C/T]TTGGAGGTGAGCCCCAACCAGGA |
| | TG |
| rs165722 | CTTCCCTGTTCTCTTCTGCTCTGTC[T/C]TCTGGTGCCCTGAGGCTGGCCTCCA |
| rs165728 | AGTGAGGATGCAGTGCTGGTTTCTG[T/C]CCACCTACACCTAGAGCTGTCCC |
| | CA |
| rs165737 | TTGGGACACCCACCCTCACGGCCTC[C/T]CCACCTGGTGCTCGCTCACCTGC |
| | AG |
| rs165774 | AAACTGGACACTGCTGTTAGCAGCC[A/G]GACTAGGAGCACGAGGGGCACA |
| | GCC |
| rs174675 | GAAATGCTGTCAGAAGCCTACCCCA[C/T]GGTACCTGTCATGGGCCTTTTCATT |
| rs174695 | GGGACAGGCGGGCACTGGGTGCCTC[C/T]TTGCACCAGCCAGGCCCAGCCT |
| | GCA |
| rs174696 | CTGCGTCCGGCCGTATTCCAGCTTT[C/T]AAAACAACAAAAAACAACAAAAACT |
| rs174697 | GGGTTCTGTGAGCATCGGAGGCACG[A/G]GGGGTGAGGGGCTCAGGAGCA |
| | GGTT |
| rs174699 | CTCCTCTGACACTGTCGCTTCTCCA[T/C]GGCATTAGATTTTCAGTCCTGCTCA |
| rs2239393 | TTACAGGAGAAGCTGTTATCACCCC[A/G]TTTCCAGGGGGCTGGGAACCCTG |
| | GG |
| rs28562050 | GAGGTTTCTCCCGATAGTGGACCCC[A/C]CGGGCCATGCCCTATGGCTGCGT |
| | GT |
| rs28565095 | TGGGGAGCCAGCTGCCTGTGCAGGT[A/G]CAAAATGGGTGGCAGAAGTGGG |
| | GTG |
| rs28621910 | CCATGCAGGTCTGGCCCATGGAAGG[C/G]AGGGGAGGGGGCCCCGACGGG |
| | GCCA |
| rs2904564 | CGGACTAGGAGCACGAGGGGCACAG[C/G]CCCCATGCCTGGCTAGGTAGG |
| | GCCG |
| rs3218737 | CGTGGGCGACAAGAAAGGTGGGGTC[C/T]GGGCCAGCAGGTGCTCAGCTCT |
| | GGG |
| rs34068292 | AGCCACTGCTCGTGCAAAGAAAGCA[C/T]GTGTCTCCTGCAGATCTTCCTCCTG |
| rs34234839 | TCCTCTGGAGTCCCAGGGTGCCAGG[G/T]TCCCTGATGACCCCTGCAGGCC |
| | CTG |
| rs34300220 | CTGGGCCCAAGCAATCCTCCCACTT[C/T]AGCCTCCTGAGTAGCTGCAGCTA |
| | TA |
| rs34446512 | GCTCTGGGCCCAGCCTGCTCTCCCC[C/G]AAGCAAGCCACTGCTCGTGCAAA |
| | GA |
| rs34450201 | GCCTTCTGCTGTCTCTTCTGAGGCC[A/G]GAAAGGCGAAGCACTGCCCTCGC |
| | CC |
| rs34691071 | GAGCTGGAGGGGGGGTCTTCCTGGG[C/T]GGCCTGTGTAGCAGCAGCTCCA |
| | GGC |
| rs34819182 | CTCTGGGACAGGGACCCAGGACCAG[G/T]CATCAAAGCCCTTACAGGAGAA |
| | GCT |
| rs34966851 | ATCTGGATTTGATGGCATTTTTATT[A/G]TGGTGTTGTTTTAGAGACAGTGTCT |
| rs35478083 | GCAGCACCAGCCCCTCTGGGCCCCA[C/T]GTGGCACAGAGTGGAAGCATCT |
| | CCT |
| rs35481270 | AGAGGCAATGGCTCAGACCAGCTCC[C/T]GCATCCCTGTAGTTGCCTCCCTG |
| | GC |
| rs35593000 | TTGCTTACAGAAGAGGCAATGGCTC[A/C]GACCAGCTCCCGCATCCCTGTAG |
| | TT |
| rs35617967 | GGGCTCTGGCCTTTGGCCTTTTCCT[C/G]TGAGCTGCATGTTGGCCACTGTG |
| | AC |
| rs35777291 | CTGAAAACATCTTGTAAGCTTTTTT[A/T]GGCCAATATATTATCAAACGCAATT |
| rs35919169 | GGACCTTGGTCATCGCCAGGTTAGG[A/G]TTTATGTTGATGATTATCCAAAGGC |
| rs36082074 | CTGGGGACAGGTGGACCCCGCAGCA[A/G]CACCAGCCCCTCTGGGCCCCAT |
| | GTG |
| rs3788319 | GTGAAGTGATCTGACGTTGGGTGGG[A/G]GTCTCCGGACTTGGGGTGGGGA |
| | ATC |
| rs3788320 | GAGGGCCTTGGTGACTTCTCCAACA[A/G]GCTCCCTGTTGGAGGCCTTGGGG |
| | TA |
| rs3810595 | TACAGCCTCTCCTTTGGCCACCCGT[C/G]ACTACCCCCAACTCCGGGCCATG |
| | GG |
| rs45454096 | ACTGGGATAGGGTGTGGGGAATTCG[A/G]ACCGCTGTGAAGTGATCTGACGT |
| | TG |
| rs4633 | CCAAGGAGCAGCGCATCCTGAACCA[C/T]GTGCTGCAGCATGCGGAGCCCG |
| | GGA |
| rs4646312 | TGGTTTGTGTATGTTCTTGGTAAAC[T/C]AGCCCTTGGTCTTACACATCATTTC |
| rs4646315 | TTCAGTCAGCCTCAGCCTCTCCAAA[C/G]AGCCAGGCATTCCAGTAGAGCCC |
| | TG |
| rs4646316 | CCCAGACCAGACACCAGGGCAGAAA[C/T]GGCACAGGACCAAGGAGATGGG |
| | GTG |
| rs4680 | CCAGCGGATGGTGGATTTCGCTGGC[A/G]TGAAGGACAAGGTGTGCATGCC |
| | TGA |
| rs4818 | GCCTGCTGTCACCAGGGGCGAGGCT[C/G]ATCACCATCGAGATCAACCCCG |
| | ACT |
| rs4986871 | ACCATCGAGATCAACCCCGACTGTG[C/T]CGCCATCACCCAGCGGATGGTGG |
| | AT |
| rs5992500 | TGCCATGGTCGGATGAGAGCAGCCA[C/T]GGACAATGTCTGTGCAATTGTGC |
| | GT |
| rs5993882 | AAAGTTACGCTTAATAATGAATGTT[T/G]CAGCACTTTCTTCTCTTCAGGTATT |
| rs5993883 | GTTGAAAGTTACTGAAAACATCTTG[G/T]AAGCTTTTTTAGGCCAATATATTAT |
| rs5993889 | GTAAGCAAACCCTTTTCTGATAGCA[G/T]AGATAGGCAAGCATCCTATGAGGTT |
| rs61910731 | TCCCCAACCCTGCACAGGCAAGATC[G/T]TGGACGCCGTGATTCAGGAGCAC |
| | CA |
| rs6267 | GCTGCAGCATGCGGAGCCCGGGAAC[G/T]CACAGAGCGTGCTGGAGGCCAT |
| | TGA |
| rs6268 | GCACCAGAGGGCACGAGAAGGCTGG[C/T]TCCCTGGCGCTGACACGTCAGG |
| | CAA |
| rs6269 | GCATTTCTGAACCTTGCCCCTCTGC[G/A]AACACAAGGGGGCGATGGTGGCA |
| | CT |
| rs6270 | CTGAGGCACTGGGGCTGGGGCCTGT[G/C]CCTTATCGGCTGGAACGAGTTC |
| | ATC |
| rs72549343 | TCCTGCAGCCCATCCACAACCTGCT[—/ |
| | A]CATGGGTGACACCAAGGAGCAGCGC |
| rs737864 | GTTGCTTTTTATTGTTTAAAGTGAC[C/T]CTCCAAGCCTGAGGGACCGTAGGAG |
| rs737865 | GCTTTTTGGATTTTTCCAGCCAGGG[A/G]TTTTTGTGTCCTGTTGCTTTTTATT |
| rs737866 | GCTAACAGACCTGCTTTTTGGATTT[C/T]TCCAGCCAGGGATTTTTGTGTCCTG |
| rs739368 | ACTGTGTGCTCCGGTTGTTGCATCC[A/G]AGGGTTGATCGGATGGTGGTTCC |
| | CA |
| rs740601 | CAGGGCCCCAAGGTGGGCGGTTCGG[G/T]GATTCAGAGAGGGCAGCTCTGT |
| | GTT |
| rs740602 | AGCATGCGGAGCCCGGGAACGCACA[A/G]AGCGTGCTGGAGGCCATTGACA |
| | CCT |
| rs740603 | TGTGAGGCACTGAGGATGCCCTCAC[G/A]CGTGCATCTGCATGTGGCGTGCA |
| | TG |
| rs769224 | ACCACTGGAAGGACCGGTACCTGCC[A/G]GACACGCTTCTCTTGGAGGTGA |
| | GCC |
| rs9306235 | GCCCATCCCAGAGGCATGTGGGGTC[A/G]GATACCAGTGTTTCAAGGCACCT |
| | GC |
| rs9332360 | AAATCCCCTAGAAGCCTGGTGTCCG[C/T]ATGACCTCCCCCTAGGGCGGAGC |
| | CT |
| rs9332367 | GGTAGGGCCGCTGGACCCTGGACAC[A/G]GATTGGAAGGAACCAGCACTAG |
| | CAG |
| rs9332377 | AACCCCTCTCCTTGGGTGCCTCTCC[C/T]TCATAGGCCTGAGTTCCTGGCACTG |
| rs9332380 | CCTGACATGCTAACCTCTCTGAACT[A/G]CAACACTGGATTGTTCTTTTTTAAG |
| rs933271 | GTGGTTACTTTCTGGAGAGAGCATG[T/C]GGCATGCAGGAGCTGGAGGGGG |
| | GGT |

The most extensively studied COMT single nucleotide polymorphism (SNP), rs4680 or Val158met, is a G to A transition that encodes a valine (Val) to methionine (Met) substitution at amino acid 158 in the membrane form of the enzyme. The Val variant is 3-4 times more enzymatically active than the Met variant. The differences in enzymatic activity are inversely correlated to endogenous levels of dopamine and other COMT substrates including epinephrine, norepinephrine and catechol estrogens. A second COMT SNP, rs4818, encodes a synonymous C to G transversion in the same exon as rs4680 and the two SNPs are in partial linkage disequilibrium. One genotype of an rs4818 polymorphism is indicated by the presence of the C allele of rs4818. Rs4818 has been associated with differential stability of COMT mRNA secondary structure as well as a series of clinical outcomes some of which are shared with rs4680 associations.

### Placebo Effects

In some embodiments placebo effects include a patient's response to inert substances, such as sugar pills, as well as the therapeutic context that surrounds medical treatment. The therapeutic encounter can include a mixture of the symbols and rituals of health care, combined with the charged emotional reactions that arise when patients encounter healers, including trust, empathy, hope, fear, trepidation, and uncertainty. In certain embodiments, by using sugar pills, saline injections, or even sham surgery, placebo research isolates medicine's provision of care from the direct effects of genuine medications or procedures. In some embodiments a clinical encounter alone—without the provision of any therapeutic treatment—can alleviate pain, improve sleep, relieve depression, and improve the symptoms of a wide variety of illnesses, including irritable bowel syndrome, benign prostatic hypertrophy, asthma, Parkinson's disease, heart disease, and migraine. In some embodiments placebos can help patients experience less fatigue, nausea, pain, and anxiety that are associated with cancer. Placebo treatment may also promote more healthful behaviors. In some embodiments, placebos can behave like drugs and the placebo effect can sometimes make drugs more effective. In some embodiments the paraphernalia of care (pills, needles, etc.) and the patient-provider relationship can be added incrementally in a manner analogous to dose dependence (the higher the dose, the greater the effect). In some embodiments a placebo effect can boost the efficacy of many powerful medications. For example, when morphine is given by injection in full view of the patient, it is almost twice as effective as when it is given through an intravenous line without the patient knowing it is being administered.

A placebo effect can be variable in its magnitude and reliability both with subjectively, patient-reported symptoms and with objectively measured symptoms. As disclosed in the Examples, variations in the COMT (catechol-O-methyltransferase) gene are correlated to placebo effects among patients with irritable bowel syndrome participating in clinical trials. A gene often included introns, exons, translated and untranslated regions. In some embodiments a gene includes gene expression regulatory regions, non-limiting examples of which include promoters, enhancers, methylated or unmethylated nucleotides, TATA box, poly A regions, regions involved in RNA stability, the like or combinations thereof. In some embodiments a gene includes translational regulatory regions non-limiting examples of which include ribosome binding sites, initiation factor binding sites, Kozak sequences, 5′-cap, ATG, CTG, stop codons, hairpin loop, palindromic sequences, regions involved in RNA stability, the like or combinations thereof. Patients with a genotype of Met/Met (e.g., having two copies of the rs4680 methionine allele) were shown to be more likely to respond to a placebo treatment, while the genotype of Val/Val (e.g., having two copies of the valine allele) responded the least. The response of patients with one copy each of methionine and valine fell in the middle. Accumulation of higher concentrations of catecholamines, such as dopamine, in patients with the Met/Met variations is thought to link to reward and ‘confirmation bias’ which enhance the sense that the treatment is working.

A similar finding presents a study where pain sensation of subjects was influenced by the cue they received describing the pain versus the actual pain delivered in form of a local heat stimuli. The difference in subjective evaluation guided by the cue and the actual experience is the equivalent of a placebo effect. Subjects of the Met/Met genotype experienced a significantly larger placebo effect in this study compared to subjects of the Val/Val genotype. Val/Met patients fell statistically in the middle of the other two polymorphism groups. (Rongjun Yu et al., (2014) Placebo analgesia and reward processing: Integrating genetics, personality, and intrinsic brain activity. Human Brain Mapping. 35: 4583-4593)

Subjects having one or more COMT polymorphisms may indicate an increased likelihood the subjects would exhibit a placebo effect. In some cases, these subjects may be excluded from the clinical trials or included in clinical trials. Additionally, the presence of one or more COMT polymorphisms in the subjects may suggest that a treatment of the subjects with the COMT polymorphism may be modified. In another embodiment, the treatment may include identifying a subject with a COMT polymorphism, modulating the treatment for the subject based presence or absence of the COMT polymorphism, and administering the modulated treatment. The modulations of the treatment may include adjusting (e.g. increasing or decreasing) a dosage or strength of the treatment administered to the subject depending on the COMT polymorphism. The “dosage,” as used herein, refers to a specified quantity of a treatment and “strength,” as used herein, refers to the concentration of a dosage. A dosage often refers to the amount of an active ingredient (e.g., an API, active pharmaceutical ingredient) in a dosage form (e.g., a pill, capsule, tab) that is administered to a subject. A treatment dosage refers to a dosage administered to a subject for treatment or prevention of a disorder, disease or condition.

### Treatments, Studies and Clinical Trials

For many classes of medications, such as analgesics, antidepressants, angina treatments, antihistamines and nonsteroidal asthma prophylaxis, well-designed, randomized, placebo-controlled trials (RCTs) often show no difference between drug and placebo. As a consequence, RCTs commonly use various ‘enrichment’ strategies that can selectively exclude participants based on pretreatment response to placebo (placebo run-in) or include them if they respond to drug (predictive enrichment). The enriched subset of patients is then randomized to drug or placebo. For example, in some embodiments, a clinical trial is a placebo-controlled clinical trial where some subjects of the trial receive at least a treatment and other subjects of the trial receive at least a placebo treatment. By depleting placebo responders—or enriching for drug responders—it is expected that the trial will show a larger drug-placebo difference, thus increasing power while decreasing sample size. Many potential threats to the validity of enrichment strategies have been proposed. For example, placebo run-in subjects may experience unblinding side effects once shifted to active drug and, conversely, patients pre-treated with drug then randomized to placebo can experience withdrawal relapse creating a bias against placebo. Probably the most severe criticism is that placebo-run-in methodologies generally fail to adequately and consistently predict patients' predispositions to having a placebo response in the clinical trial.

Given the limitations of current enrichment strategies, coupled with the recent increases in clinical trial costs and placebo-response rates, identifying placebo-response biomarkers to guide enrichment could prove to be a valuable strategy. COMT genetic variants are an efficient and inexpensive way of potentially identifying a significant proportion of placebo responders, thus potentially greatly reducing the clinical time and resources involved in treatment-based enrichment strategies. For example, the exclusion of genetically designated high placebo responders—such as, COMT rs4680 Met/Met—and/or the inclusion of low placebo responders—such as, Val/Val can greatly reduce the clinical time and resources involved in treatment-based enrichment strategies. Heterozygous genetic variants may represent the largest group; and have an intermediate level of placebo response. Inclusion of COMT genetic variants would be expected to decrease effect sizes of clinical trials. The “effect size,” as used herein, refers to the number of subjects needed in a clinical trial to demonstrate statistical significance of active treatment efficacy over placebo response.

In one aspect, a method of selecting subjects to participate in a clinical trial includes identifying subjects with a catechol-O-methyltransferase (COMT) polymorphism, where the COMT polymorphism modulates a placebo effect in the subjects, and selecting subjects to participate in the clinical trial based on their COMT polymorphism. The COMT polymorphism can be at least one of rs4680, rs4818, rs6269, and rs4633. The COMT polymorphism can also encode a valine/methionine haplotype, a methionine/methionine haplotype, or a valine/valine haplotype. In one embodiment, the COMT polymorphism indicates an increased likelihood the subjects would exhibit a placebo effect. By identifying subjects with the COMT polymorphism, those subject may be excluded or included from a clinical trial or a treatment may be modified depending on the COMT polymorphism.

In another aspect patients with a certain disorder are evaluated in a clinical study for their placebo response and a correlation is established between high placebo responders and their respective COMT polymorphism haplotype. The COMT haplotype, e.g. COMT rs4680 Met/Met or COMT rs4680 Val/Val, that demonstrated the highest correlation to an observed placebo effect is effectively determined to be a predictor for placebo response in the evaluated indication. Patients of this COMT haplotype type can be excluded from future clinical studies for the purpose of reducing the placebo rate in RCTs. The COMT polymorphism can be at least one of rs4680, rs4818, rs6269, and rs4633. The COMT polymorphism can also encode a valine/methionine haplotype, a methionine/methionine haplotype, or a valine/valine haplotype.

In another aspect, after the exclusion of patients with a COMT polymorphism haplotype correlated to high placebo response, the remaining patients are randomized in the various, comparative treatment arms and placebo arms of the clinical trials in ways that result in an equal distribution of the remaining COMT haplotypes in each arm in order to achieve best possible equalization of treatment responders and placebo responders in all arms of the study. The COMT polymorphism can be at least one of rs4680, rs4818, rs6269, and rs4633. The COMT polymorphism can also encode a valine/methionine haplotype, a methionine/methionine haplotype, or a valine/valine haplotype.

In another aspect, patients are randomized in the various, comparative treatment arms and placebo arms of the clinical trials in ways that result in an equal distribution of the COMT haplotypes in each arm in order to achieve best possible equalization of treatment responders and placebo responders in all arms of the study. The COMT polymorphism can be at least one of rs4680, rs4818, rs6269, and rs4633. The COMT polymorphism can also encode a valine/methionine haplotype, a methionine/methionine haplotype, or a valine/valine haplotype.

In some embodiments a treatment is administered to a subject. In certain embodiments a treatment is administered to one or more subjects having, suspected of having or at risk of having a disorder, ailment, disease or condition. In certain embodiments a treatment is administered to a subject or group of subjects as part of a clinical trial. In certain embodiments a treatment is administered to a subject or group of subjects selected as a test group or study group. In some embodiments all subjects of a clinical trial, test group or study group are administered a treatment. In some embodiments some subjects, or selected subjects of a clinical trial, test group or study group are administered a treatment. A treatment can comprise administering one or more compounds and/or drugs to one or more subjects. A treatment can comprise administering a therapy to one or more subjects. Non-limiting examples of a therapy include a surgery (minor or major, invasive or non-invasive), physical therapy, administering a physical stimuli (e.g., acupuncture, electrical treatment, sonic treatment, radiation treatment and the like). Any suitable, new, known or experimental compound, drug or therapy can be administered to a sub-population, sub-group, study group or human subject selected by a method described herein. Suitable doses and methods of administering new, known or experimental compounds, drugs and/or therapies to human subjects are often determined (e.g., pre-determined) by scientists and/or medical professionals, which doses and methods are often specific for a particular disorder, ailment, disease or condition.

In some embodiments a subject or group is administered a treatment. Any suitable treatment or experimental treatment can be administered. In some embodiments a treatment comprises administering vitamin E and/or aspirin and the response is a clinical characteristic of cardiovascular disease. A clinical characteristic of cardiovascular disease can be selected from a frequency or degree of myocardial infarction and/or a frequency or degree of stroke. In some embodiments a clinical characteristic of cardiovascular disease is selected from one or more of a systolic blood pressure, diastolic blood pressure, or an amount of serum triglycerides, serum apolipoprotein B and serum soluble intracellular adhesion molecule I.

In some embodiments a treatment comprises administering an analgesic and the measured response is a sensation of pain.

In some embodiments a treatment comprises administering a pharmaceutical composition for the treatment of irritable bowel syndrome and the measured response is a clinical characteristic of irritable bowel syndrome. In certain embodiments a clinical characteristic of irritable bowel syndrome is selected from the group consisting of abdominal pain severity, abdominal pain frequency, abdominal distention severity, dissatisfaction with bowel habits, and disruption in quality of life.

In some embodiments a treatment comprises administering a drug that interacts with an opioid pathway involved with a placebo response. In certain embodiments a drug that interacts with an opioid pathway is naloxone, a pain medication, ketorolac, an opiate, or buprenorphine.

In some embodiments a treatment comprises administering a drug that interacts with a serotonin pathway. In some embodiments a drug that interacts with a serotonin pathway is a selective serotonin reuptake inhibitor or a tricyclic.

In some embodiments a treatment comprises administering a drug that binds directly to a COMT protein. In some embodiments a treatment comprises administering a drug that inhibits an activity or function of a COMT protein. In some embodiments a treatment comprises administering a drug is selected from quercetin, S-adenosylmethionine, tolcapone, entacapone, a beta-blocker, an alpha adrenergic receptor blocker, a beta-adrenergic receptor blocker, a agonist of alpha adrenergic receptor blocker, a agonist of beta-adrenergic receptor blocker, vitamin A, vitamin C, vitamin D, vitamin E and levodopa.

In some embodiments a treatment comprises administering a pharmaceutical composition for the treatment of irritable bowel syndrome, diabetes, an autoimmune disorder, inflammation, a neurological disorder, chronic and acute pain, cancer, allergies, depression, migraines, addiction, obesity or cardiovascular disease.

A treatment is sometimes an experimental treatment. In some embodiments an experimental treatment comprises evaluating a treatment (e.g., drug and/or therapy) that is new or experimental. In certain embodiments the efficacy of a treatment is determined according to the results (e.g., responses of subjects) obtained from testing a treatment. In certain embodiments the efficacy of a treatment is determined according to the results obtained from a clinical trial and/or experimental study. In certain embodiments the effective dosing, effective routes of administration, efficacy, and/or adverse effects (e.g., side effects) of a treatment are determined according to the results of a clinical trial and/or experimental study. A response to a treatment is often compared between two or more subjects or between two or more groups of subject, some of which are administered the treatment. A response to a treatment can be determined by any suitable method. In some embodiments, determining a response to a treatment comprises comparing a response of a first group of subjects who are administered a treatment to the response of a second group of subjects who are not administered the treatment. Comparing a response of multiple groups of subjects can be complicated and often involves sophisticated statistical analysis by methods known in the art. A response to a treatment can be a positive response, a neutral response or a negative response. For example, a positive response may be a response that is expected and/or a response that indicates a positive change in a subjects condition (e.g., an improvement of one or more symptoms or physiological responses). As described herein, a response of a subject to a treatment can often depend, at least in part, upon the genotype of the subject.

Efficacy of a treatment refers to the ability of a treatment (e.g., a drug) to produce one or more desired responses in a subject. For example, the efficacy of a drug is sometimes determined by measuring a response in two or more groups of subjects, where at least a first group is administered the drug and at least a second group does not receive the drug, and comparing the measured response for the at least first and second groups.

In certain embodiments, subjects of a study (e.g., a clinical trial) are arranged into subgroups or blocks according to genotype. In some embodiment, each genotype is determined according to the presence or absence of specific PAPs identified and defined herein.

In certain embodiments evaluating or assessing the results of a treatment comprises determining the presence, absence, description and/or degree of one or more subjective symptoms. The presence, absence, description and/or degree of one or more subjective symptoms can be determined prior to, during or after administration of a treatment. Testing a treatment often comprises evaluating the efficacy of a treatment to reduce or alleviate one or more subjective symptoms.

In certain embodiments a treatment is administered as part of a clinical trial, clinical study or medical study. In some embodiments, administering a treatment comprises administering an experimental drug or therapy. In certain embodiments a treatment is administered to one or more study groups. Some or all subjects of a sub-group or study group, or in certain embodiments some or all sub-groups or study groups may receive a treatment. In some embodiments, a treatment is not administered to all subjects, sub-groups or study groups of a clinical study or medical study.

In some embodiments a placebo treatment is administered. A placebo treatment can be any simulated or otherwise medically ineffectual treatment administered to a subject. A placebo can be any compound, inert ingredient, inert substance or composition that is known or expected to have little or no pharmacological effect on a disorder, disease or condition. In some embodiments a placebo comprises a dosage form (e.g., a pill, tablet, capsule or liquid). In some embodiments a placebo comprises a suitable pharmaceutical excipient or diluent. A placebo often does not contain an active pharmaceutical ingredient (API). In some embodiments a placebo comprises a medically ineffectual substance, non limiting examples of which include a sugar, a starch, one or more amino acids, a vitamin, a salt, water, a lipid, a food substance, the like or a combination thereof. A placebo can be administered at any suitable dosage, amount, weight or volume. In certain embodiments a placebo treatment is administered orally. In certain embodiments, a placebo treatment comprises administering a dosage form. In certain embodiments a placebo treatment comprises administering a placebo tablet, granule, liquid, capsule, pill, or the like wherein the tablet, granule, liquid, capsule or pill contains an inert ingredient (e.g., ineffective ingredient). A placebo can be administered by any suitable method which is often the same method used to administered an experimental drug or experimental compound that is being evaluated. A placebo can be self-administered or administered by another. In some embodiments a placebo treatment comprises a mock surgery or mock treatment.

In certain embodiments conducting a clinical or medical trial comprises testing a treatment (e.g., an experimental treatment). In some embodiments a method of testing a treatment comprises administering a treatment to some or all subjects, sub-groups, test groups or study groups and administering a placebo treatment to some or all subjects, sub-groups, test groups or study groups. In certain embodiments a method of testing a experimental treatment comprises administering a treatment to some subjects, sub-groups, test groups or study groups and administering a placebo treatment to other subjects, sub-groups, test groups or study groups where the subjects, sub-groups, test groups or study groups that receive the experimental treatment are different subjects, sub-groups, test groups or study groups than those that received the placebo treatment. In some embodiments a method of testing a treatment comprises excluding some subjects, sub-groups, test groups or study groups from receiving either a treatment or a placebo treatment. In certain embodiments conducting a clinical or medical trial comprises excluding some subjects, sub-groups, test groups or study groups from participating in a clinical or medical trial.

In certain embodiments a study (e.g., a medical study, a clinical trial) comprises testing a treatment (e.g., an experimental drug) where a sub-population of subjects is first selected for participation in the study. Subjects of such sub-population are often selected because they have a particular disorder, disease or condition and/or because they are in need of a treatment for a particular disorder, disease or condition. In certain embodiments a sub-population of subjects are selected for participation in a study because they have a particular disorder or disease and the study is designed to test a treatment for the particular disorder or disease. Sometimes subjects of a sub-population of subjects are selected by a random method. In some embodiments, subjects of a sub-population of subjects are selected for participation in a clinical trial according to having one or more genotypes of one or more COMT polymorphisms.

In certain embodiments, a sub-population of subjects are tested for having a specific genotype of one or more COMT polymorphisms. In certain embodiments, the genotype of a COMT polymorphism is detected for a group of human subjects (e.g., a sub-population). A sub-population of subjects can be further segregated or divided into two or more sub-groups and/or study groups according to a genotype of one or more COMT polymorphisms detected. In some embodiments, subjects of a sub-population that are determined to have one or more COMT polymorphisms are excluded from a study or clinical trial. In some embodiments, subjects of a sub-population that are determined to have a specific genotype of one or more COMT polymorphisms are excluded from one or more study groups or test groups of a study. For example, in certain embodiments a sub-group of subjects determined to have a specific genotype of a COMT polymorphism are excluded from one or more, or all study groups of a study (e.g., study groups of a medical or clinical trial). Subjects excluded from a study are often not administered a treatment of any kind (e.g., an experimental treatment or placebo treatment). In certain embodiments, subjects of a subgroup that are determined to have a specific genotype of a COMT polymorphisms are distributed among two or more study groups in an attempt to normalize or minimize placebo effects on a medical or clinical study. In certain embodiments, subjects of a subgroup that are determined to have a specific genotype of a COMT polymorphisms are distributed equally or about equally among at least two study groups in an attempt to normalize or minimize placebo effects on a medical or clinical study. In some embodiments, subjects of a sub-group that have a specific genotype of a COMT polymorphism and subjects that do not have a different genotype of a COMT polymorphism are distributed equally or about equally among at least two study groups in an attempt to normalize or minimize placebo effects. In some embodiments, distributing subjects about equally among one or more groups, sub-groups or study groups includes a random distribution of the subjects, where the distribution is not always an equal distribution. In some embodiments, distributing subjects about equally among groups comprises a randomization method and any suitable randomization method can be used (e.g., a blocked randomization method).

In certain embodiments, subjects that are homozygous and/or heterozygous for a specific genotype of a COMT polymorphism are excluded from a study. For example, in some embodiments subjects that are homozygous for an A allele of an rs4680 polymorphism are excluded from a study. In some embodiments subjects that are heterozygous for an A allele of an rs4680 polymorphism are excluded from a study. In some embodiments subjects that are heterozygous and subjects that are homozygous for an A allele of an rs4680 polymorphism are excluded from a study. In certain embodiments, subjects that are homozygous for the absence of a COMT polymorphism are excluded from a study.

In certain embodiments, subjects that are homozygous and/or heterozygous for the presence of a specific genotype of a COMT polymorphism are distributed among study groups of a study (e.g., study groups that receive a placebo treatment, study groups that receive a treatment, control groups and the like). For example, in some embodiments subjects that are homozygous for an A allele of an rs4680 polymorphism are identified and distributed among the study groups that are used for a study. In some embodiments subjects that are heterozygous for an A allele of an rs4680 polymorphism are identified and distributed among the study groups that are used for a study. In some embodiments subjects that are heterozygous and subjects that are homozygous for an A allele of an rs4680 polymorphism are identified and distributed among the study groups that are used for a study. Subjects of a sub-group can be distributed by any suitable method among study groups of a study. In certain embodiments, subjects that are homozygous for the absence of a COMT polymorphism are distributed among study groups of a study For example, subjects of a sub-group can be distributed randomly, evenly and/or equally among study groups of a study.

Each study group or test group of a study is often designated to receive one or more treatments. For example some study groups may be administered a treatment, some may receive a placebo treatment and some may not receive a treatment. In some embodiments, a first study group receives a first treatment and a second study group is administered the first treatment and a second treatment. The design of certain clinical and/or medical studies can be quite complex involving 2 or more, 3 or more, 5 or more, 10 or more or 20 or more study groups and may involve at least 1, 2, 3, 4, 5 or six or more treatments.

In the case of irritable bowel syndrome, diabetes, autoimmune disorders, inflammation, neurological disorders, acute pain, chronic pain, cancer, cancer treatments, allergies, depression, migraines, addiction, obesity, cardiovascular disorders and other disorders, syndromes, or diseases, the placebo responders identified by their COMT genotype could receive less of the active pharmacological treatment thus limiting their negative side-effects while still maintaining their placebo response. Alternatively patients identified as non-placebo responders may be candidates for a stronger more focused pharmacological intervention. In the case of clinical conditions with more objective outcomes, such as Parkinson's, asthma, addiction, arthritis, angina and diabetes treatment of placebo responders with drugs that interact with dopamine or other catecholamine pathways could be modified based on the interaction between the treatment and the patient's COMT polymorphism in order to mitigate potential negative interactions or promote positive interactions between the treatment and the catecholamine mediated pathway.

In some embodiments a treatment comprises administering a beta blocker. Beta blockers (β-blockers, beta-adrenergic blocking agents, beta antagonists, beta-adrenergic antagonists, beta-adrenoreceptor antagonists, or beta adrenergic receptor antagonists) comprise a class of drugs that are often used for the management of cardiac arrhythmias, protecting the heart from a second heart attack (myocardial infarction) after a first heart attack (secondary prevention), and, in certain cases, hypertension. Beta blockers often block the action of endogenous catecholamines epinephrine (adrenaline) and norepinephrine (noradrenaline)—in particular on adrenergic beta receptors, of the sympathetic nervous system, which mediates the fight-or-flight response. Some beta blockers block all activation of β-adrenergic receptors and others are selective. At least three types of beta receptors are known, designated β1, β2 and β3 receptors. β1-adrenergic receptors are located mainly in the heart and in the kidneys. β2-adrenergic receptors are located mainly in the lungs, gastrointestinal tract, liver, uterus, vascular smooth muscle, and skeletal muscle. β3-adrenergic receptors are located in fat cells. Beta receptors are often found on cells of the heart muscles, smooth muscles, airways, arteries, kidneys, and other tissues that are part of the sympathetic nervous system and lead to stress responses, especially when they are stimulated by epinephrine (adrenaline). Beta blockers often interfere with the binding to the receptor of epinephrine and other stress hormones, and weaken the effects of stress hormones.

In some embodiments a treatment comprises administering an alpha-blocker (e.g., α-blockers, or α-adrenergic-antagonists). Alpha-blockers are often pharmacological agents that act as receptor antagonists of α-adrenergic receptors (α-adrenoceptors). Alpha-blockers can be α1-blockers or antagonists that act at α1-adrenoceptors or α2-blockers or antagonists that act at α2-adrenoceptors. An alpha blocker can sometimes be an α1-blockers, an α2-blocker or an agents that act at both types of receptors. Non-limiting examples of non-selective alpha-blockers include Phenoxybenzamine, Phentolamine, Tolazoline, Trazodone, antipsychotics, Alfuzosin, Prazosin, Doxazosin, Tamsulosin, Terazosin, Silodosin, Atipamezole, Idazoxan, Mirtazapine, Yohimbine, carvedilol and labetalol. Alpha-Blockers are often used in the treatment of several conditions, such as Raynaud's disease, hypertension, and scleroderma. Alpha-blockers can also be used to treat anxiety and panic disorders, such as generalized anxiety disorder, panic disorder, or posttraumatic stress disorder (PTSD). While most commonly used to treat hypertension (usually in conjunction with diuretics when other treatments are ineffective), they are also often used to treat the symptoms of BPH (benign prostatic hyperplasia).

In some embodiments a treatment comprises administering an adrenergic alpha-agonist (e.g., an alpha-adrenergic agonists). Adrenergic alpha-agonists are a class sympathomimetic agents that selectively stimulates alpha adrenergic receptors. The alpha-adrenergic receptor has two subclasses α1 and α2. Alpha 2 receptors are associated with sympatholytic properties. Adrenergic alpha-agonists often have the opposite function of alpha blockers. Adrenergic alpha-agonists often mimic the action of epinephrine and norepinephrine signaling in the heart, smooth muscle and central nervous system.

In some embodiments a treatment comprises administering a beta-adrenergic agonists (e.g., beta-agonist). Beta-adrenergic agonists are often medications that relax muscles of the airways, which widens the airways and results in easier breathing. They are a class of sympathomimetic agents which act upon the beta adrenoceptors. Pure beta-adrenergic agonists often have the opposite function of beta blockers. Beta adrenoreceptor agonist sometimes mimic the action of epinephrine and norepinephrine signaling in the heart, lungs and smooth muscle tissue, with epinephrine being the highest affinity. The activation of β1, β2 and β3 can activate the enzyme, Adenylate cyclase. This in turn often leads to the activation of the secondary messenger Cyclic adenosine monophosphate and induces smooth muscle relaxation and contraction of the cardiac tissue. Activation of β1 receptors can induce positive inotropic, chronotropic output of the cardiac muscle, leading to increased heart rate and blood pressure, secretion of ghrelin from the stomach, and renin release from the kidneys. Activation of β2 receptors can induce smooth muscle relaxation in the lungs, gastrointestinal tract, uterus, and various blood vessels. Increased heart rate and heart muscle contraction is also associated with the β2 receptors. β3 receptors are often located in adipose tissue. Activation of the β3 receptors can induce the metabolism of some lipids. Beta-adrenergic agonists can be used to treat bradycardia (slow heart rate), asthma, chronic obstructive pulmonary disease (COPD), heart failure, allergic reactions, and hyperkalemia for example.

In some embodiments, methods for treating a subject are disclosed. One embodiment can include determining the presence or absence of a PAP (e.g., a catechol-O-methyltransferase (COMT) polymorphism) in the subject, identifying a treatment for the subject based on the presence or absence of the PAP, and administering the treatment to the subject. The method can also include obtaining a nucleic acid sample from the subject and analyzing the nucleic acid sample for a PAP. The method can include obtaining a sample, such as a blood, urine, ascites, cerebrospinal fluid, bronchial lavage, oral washings and sputum, Pap smears, tissue biopsies or organs, bile, fecal matter, or other bodily fluids, tissues or parts from the subject and analyzing the sample for the presence or absence of a PAP.

Also disclosed is a method for determining a treatment dosage by determining the presence of absence of a PAP (e.g., a catechol-O-methyltransferase (COMT) polymorphism) in a subject and modulating the treatment dosage administered to the subject based on the COMT polymorphism. The subject can be in a clinical trial and the dosage can be modulated depending on the presence or absence of a PAP. The method can also include obtaining a nucleic acid sample from the subject and analyzing the nucleic acid sample for the presence or absence of a PAP. The method can include obtaining a sample, such as a blood, urine, ascites, cerebrospinal fluid, bronchial lavage, oral washings and sputum, Pap smears, tissue biopsies or organs, bile, fecal matter, or other bodily fluids, tissues or parts from the subject and analyzing the sample for the presence or absence of a PAP.

Identification of a treatment can include modulating a treatment for a subject according to the presence or absence of a PAP (e.g., a COMT polymorphism) and administering the modulated treatment and/or identifying an alternative treatment for the subject according to the presence or absence of a PAP, and administering the treatment to the subject. In some embodiments, a treatment can be adjusted by administering a modulated treatment (e.g., increased, decreased treatment), by ceasing treatment or by including another treatment depending on the presence or absence of a PAP. An treatment can be increased or decreased. Increasing a treatment often refers to increasing an amount of an API administered to a subject. A treatment may be increased from 1% to 1000% or more. For example a treatment may be increased by 1%, 10%, 50%, 100%, 200%, 300%, 400%, 500%, 1000% or more. Decreasing a treatment often refers to decreasing an amount of an API administered to a subject. A treatment may be decreased by 1% to 1000% or more. For example a treatment may be decreased by 1%, 10%, 50%, 100%, 200%, 300%, 400%, 500%, 1000% or more. In some embodiments a treatment is increased or decreased relative to an amount of treatment prescribed or administered prior to a determination of the presence or absence of a PAP. In some embodiments a treatment is increased or decreased relative to a dosage previously prescribed or administered to a subject (e.g., a dosage prescribed or administered by a medical professional, e.g., a physician, nurse, physician's assistant) prior to a determination of the presence or absence of a PAP. In some embodiments a treatment is increased or decreased relative to a dosage indicated for a specific disorder, disease or condition. An API is often indicated (e.g., approved) for use to treat a specific disorder, disease or condition by the Food and Drug Administration within a specified dosage range and the API is said to be indicated for that particular disorder, disease or condition. However, the term “indicated for” as used herein also refers to an API and dosage amount thereof that is commonly used to treat a specific disorder, disease or condition, even though the use thereof is considered as an “off label” use.

In some embodiments one, two, or three genotypes of a placebo-associated polymorphism are detected in a sub-population of human subjects, wherein the human subjects have or are suspected of having a disorder or condition. Subjects can be distributed evenly, unevenly or randomly among two or more study group. In some embodiments subjects are distributed among two or more subgroups according to genotype where each subgroup consist of only subjects of a single genotype of a PAP. Sometimes subjects are distributed among two or more subgroups or study groups according to genotype where subjects with different genotypes of the same PAP are distributed evenly, unevenly or randomly among the study groups. One or more subjects of one or more study groups are often administered a treatment. Sometimes one or more subjects of one or more study groups are often administered a placebo treatment. Sometimes one or more subjects of one or more study groups receive no treatment. In some embodiments one or more treatments are combined and are evaluated independently and or together by administering the one or more treatments alone or in combination to one or more study groups. Responses are often determined and/or measured for subjects in one or more study groups receiving one or more treatments, no treatment or one or more placebo treatments. Responses are often compared and evaluated to determined efficacy and/or safety. Sometimes responses to a placebo are determined according to a disorder or condition of a subject. For example, a study may look for an improvement in clinical characteristics of a disease or improvement of one or more symptoms associated with a disease or disorder for subjects administered a placebo treatment, no treatment, a treatment or a combination thereof. Often when one or more subjects of a first study group having a first genotype of a PAP show an improvement in clinical characteristics of a disease or improvement of one or more symptoms associated with a disease when compared to another study group having a different genotype of a PAP, where the two study groups received the same treatments (e.g., a placebo treatment or treatment), the improved response indicates an enhanced response due to the genotype of the first study group compared to the genotype of the other study group.

Diseases

Studies investigating brain activity associated with placebo response in pain point to catecholamines such as dopamine as a possible integrator of the placebo response. Dopamine and other catecholamine are packaged into presynaptic vesicles and released into the synaptic cleft upon depolarization. Dopamine is cleared from the synapse either by the dopamine reuptake transporter (DAT), or degradation by monoamine oxidases A and B, or catechol-O-methyltransferase (COMT). Whereas reuptake is the primary mechanism of dopamine clearance in the striatum, in the prefrontal cortex, where monoamine oxidase and DAT is less abundant, COMT activity is critical in regulating prefrontal dopamine and other catecholamine signaling.

### Irritable Bowel Syndrome (IBS)

Irritable bowel syndrome (IBS) is a common gastrointestinal disorder affecting 10 to 15% of North Americans is characterized by abdominal pain or discomfort associated with altered bowel function, bloating, and a sensation of incomplete evacuation after bowel movements. IBS is a condition known to have a high placebo response rate and meta-analyses report an average placebo induced global improvement of approximately 40%.

The COMT allele, such as the Met/Met genotype, can be a potential marker for placebo responders in disease. The number of COMT major or minor alleles may correspond to COMT activity. This may, in turn, correspond to dopamine or other catecholamine availability in the prefrontal cortex that would relate to placebo responses. Further, a relationship may exist between number of a given type of COMT allele and placebo response. Furthermore the finding that particular genotypes are associated with a particular outcome, positive or negative, in groups administered a placebo treatment can be a predictor of a placebo effect.

In one aspect, a subject with irritable bowel syndrome, being of the COMT haplotype Met/Met may receive traditional pharmacological treatment with augmented placebo treatment facilitated by the therapeutic encounter with or without non-traditional, non-pharmacological interventions such as acupuncture, massage, placebo pills or similar.

Another aspect can include the tapering off of traditional pharmacological treatments during or after a placebo or non-pharmacological treatment administration in patients with the COMT polymorphism haplotype that responds favorably to placebo treatment.

In one aspect, treating a subject who has, is suspected of having, or is at risk for developing irritable bowel syndrome is disclosed. The method can include determining a catechol-O-methyltransferase (COMT) polymorphism in the subject, identifying a treatment for the subject based on the COMT polymorphism, and administering the treatment to the subject.

Another aspect can include determining a treatment dosage for a subject with, suspected of having, or at risk for developing irritable bowel syndrome. The catechol-O-methyltransferase (COMT) polymorphism in a subject can be determined, and the treatment dosage administered to the subject can be modulated based on the COMT polymorphism. The modulation can include increasing or decreasing the dosage for the subject with the COMT polymorphism, identifying an alternative treatment for subject with the COMT polymorphism.

Functional diseases such as IBS, chronic fatigue and Fibromyalgia; diseases related to the pathophysiology of dopamine such as depression and schizophrenia; and disease related to the pathophysiology of other catecholamines including cardiovascular and metabolic diseases such as coronary artery disease and diabetes; may be affected by the COMT mediated placebo response. Screening patients for their COMT polymorphism and selecting the group that responds most favorably to placebo enables the caregiver to augment the pharmacological treatment effect with placebo treatments or non-pharmacological treatments that would elicit an additive placebo effect in these patients. Drug dosages and treatment strengths may be tapered down in these patients.

### Acute or Chronic Pain

Acute or chronic pain as results of injury, surgery or disease is experienced at some point in life by every person. Effective analgesics are available, however they have limitations due to tolerability issues, side effects and dependency issues. For this reason many patients, especially those with chronic pain are underserved. The presented data suggest that pain sensation is highly susceptible to placebo with subjects of the Met-Met haplotype responding significantly more to placebo stimuli. Successful augmentation of pain treatment with placebo, including adjustment of the therapeutic encounter, or non-pharmacological treatments is therefore a feasible alternative to increasing doses or increasing strength of pain medication. Such placebo augmented treatment would spare the patients the side effects of higher doses or stronger pain medication and may even allow tapering of the medication.

### Other Disorders, Syndromes or Diseases

In one aspect, treating a subject with, suspected of having, or at risk for developing at least one of autoimmune disorders, inflammation, neurological disorders, chronic pain, cancer, cancer treatments, allergies, depression, migraines, schizophrenia, addiction, obesity, and any combination of other disorders, syndromes, and diseases is disclosed. Another aspect can include determining a treatment dosage for a subject with, suspected of having, or at risk for at least one of autoimmune disorders, inflammation, neurological disorders, schizophrenia, chronic pain, cancer, cancer treatments, allergies, depression, migraines, addiction, obesity, and any combination of other disorders, syndromes, and diseases. In some embodiments a subject has or is suspected of having a disorder selected from the group of irritable bowel syndrome, diabetes, an autoimmune disorder, inflammation, a neurological disorder, chronic and acute pain, cancer, allergies, depression, migraines, addiction, obesity, and cardiovascular disease. In some embodiment a neurological disorder comprises pain, depression, and/or psychosis.

### Detection Methods, Assays and Kits

In one aspect, methods and materials for a diagnostic kit and assay are included to determine the COMT polymorphism. The kit or assay can include materials for obtaining a nucleic acid sample from the subject and reagents for analyzing the nucleic acid sample for the COMT polymorphism. The kit can also include materials for obtaining a sample, such as a blood, urine, ascites, cerebrospinal fluid, bronchial lavage, oral washings and sputum, Pap smears, tissue biopsies or organs, bile, fecal matter, or other bodily fluids, tissues or parts from the subject and analyzing these samples for the COMT polymorphism.

The presence or absence of a COMT polymorphism can be detected by a suitable method, assay or by a method described herein. In some embodiments a COMT polymorphism is detected at the genetic level (e.g., detection of a COMT polymorphism in genomic DNA or mRNA) using a suitable technique. In some embodiments a COMT polymorphism is detected at the protein level (e.g., detection of a COMT polymorphism in COMT protein) using a suitable technique. For example, in some aspects, a protein or peptide assay for determining a COMT polymorphism in a subject can be used. In certain embodiments, detecting the presence or absence of a COMT polymorphism comprises detecting an amino acid mutation (e.g., an amino acid substitution, deletion or insertion) in a COMT polypeptide. In certain embodiments, detecting the presence of a COMT polymorphism comprises detecting the presence of methionine 158 in a COMT polypeptide and/or detecting the absence of valine 158 in a COMT polypeptide. In certain embodiments, detecting the absence of a COMT polymorphism comprises detecting the absence of methionine 158 in a COMT polypeptide and/or detecting the presence of valine 158 in a COMT polypeptide. In certain embodiments, a COMT polymorphism is detected with the use of at least one antibody specific for a COMT polymorphism. In certain embodiments a kit for detecting a COMT polymorphism comprises at least one antibody specific for detecting a COMT polymorphism. The assay can also include reagents for measuring or comparing catechol-O-methyltransferase enzymatic activity. The assay can further include reagents for analyzing catechol-O-methyltransferase protein. Non-limiting example of methods that can be used to detect a COMT polymorphism include immunohistochemistry, a suitable immune assay (e.g., an enzyme-linked immunosorbent assay (ELISA)), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APP I-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassay, microscopy, microfluidic chip-based assays, surface plasmon resonance, sequencing, Western blotting assay, the like or a combination thereof. Thus a kit or assay for detection of a COMT polymorphism can include reagents specific for any of immunohistochemistry, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassay, microscopy, microfluidic chip-based assays, surface plasmon resonance, sequencing, Western blotting assay, or a combination thereof. In some embodiments a COMT polymorphism is detected by analyzing the expression, modifications and/or conformational changes of a COMT protein or variant thereof (e.g., a variant resulting from a polymorph). For example, in certain embodiments an assay can be configured to detect phosphorylation, glycosylation, carboxylation, methylation, lipid modification, ubiquitination, myristoylation, conformation, conformational changes in protein folding, monomerization and/or polymerization, or other conformational states of a COMT protein. In some embodiments a kit or assay can comprise reagents for detecting modifications and/or conformational changes of COMT protein or other biomarkers, such as phosphorylation, glycosylation, methylation, lipid modification, ubiquitination, myristoylation state, conformational change in protein folding, monomerization and/or polymerization, and other conformational states of the protein.

A suitable sample can be obtained from a subject for analysis. The sample can include, for example, nucleic acids, proteins, peptides, precursors, lipids, carbohydrates, metabolites, and other COMT polymorphism biomarkers to be separated/isolated/purified from bodily fluids, cells, or tissues of a subject. Any suitable method can be used to isolate, separate, and/or purify a COMT protein, mRNA or DNA. Cell separation/isolation/purification methods can be used to isolate a diagnostic sample for use with a diagnostic kit. A skilled artisan can use any known cell separation/isolation/purification techniques to isolate the diagnostic sample from the subject's sample. Exemplar techniques include, but are not limited to, using antibodies, flow cytometry, fluorescence activated cell sorting, filtration, gradient-based centrifugation, elution, microfluidics, magnetic separation technique, fluorescent-magnetic separation technique, nanostructure, quantum dots, high throughput microscope-based platform, or a combination thereof.

In certain aspects described herein, analytes include nucleic acids, proteins, lipids, carbohydrates, metabolites, or any combinations of these. In certain aspects of the methods described herein, biomarkers include nucleic acids, proteins, precursors, lipids, carbohydrates, metabolites, or any combinations of these. As used herein, the term “nucleic acid” is intended to include DNA molecules (e.g., cDNA or genomic DNA), RNA molecules (e.g., mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNAs), microRNA (miRNAs), small nucleolar RNA (snRNAs), ribosomal RNA (rRNA), transfer RNA (tRNA), small interfering RNA (siRNA), heterogeneous nuclear RNAs (hnRNA), or small hairpin RNA (shRNA).

A kit (e.g., a diagnostic kit) can be used to detect the presence, absence, or a difference in genetic alleles. In some embodiments a kit can include suitable nucleic acids or suitable reagents (e.g., at least one primer or probe specific for a COMT polymorphism and hybridization reagents, e.g., one or more primers configured to amplify a portion of a gene or cDNA suspected of having a COMT polymorphism) to determine the presence or absence of a COMT polymorphism in a subject. In certain embodiments, a difference between different profiles detected can refer to different gene copy numbers, genetic alleles, different DNA, RNA, proteins, lipids, or carbohydrate expression levels, different DNA methylation states, different DNA acetylation states, and different protein modification states that result from different genetic profiles.

As used herein, a “profile” of a marker, e.g. COMT, of a disease or condition can broadly refer to any information concerning the marker. This information can be either qualitative (e.g., presence or absence) or quantitative (e.g., levels, copy numbers, or dosages). In some embodiments, a profile of a marker can indicate the absence of this marker. The profile can be a nucleic acid (e.g., DNA or RNA) profile, a protein profile, a lipid profile, a carbohydrate profile, a metabolite profile, or a combination thereof. A “marker” as used herein generally refers to an analyte which is differentially detectable and is indicative of the presence of a disease or condition. An analyte is differentially detectable if it can be distinguished quantitatively or qualitatively. Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

A nucleic acid profile can be, without limitation, a genotype, a genotypic profile, a single nucleotide polymorphism profile, a gene mutation profile, a gene copy number profile, a DNA methylation profile, a DNA acetylation profile, a chromosome dosage profile, a gene expression profile, or a combination thereof.

A COMT polymorphism and/or a nucleic acid profile can be determined by a suitable method known in the art to detect genotypes, single nucleotide polymorphisms, gene mutations, gene copy numbers, DNA methylation states, DNA acetylation states, chromosome dosages and the like. In certain embodiments, detecting the presence or absence of a COMT polymorphism comprises detecting a polymorphism in one or more alleles of a COMT gene. In certain embodiments, detecting the presence of a COMT polymorphism comprises detecting the presence or absence of one or more polymorphisms of Table 1 in a COMT gene. In certain embodiments, detecting the presence of a COMT polymorphism comprises detecting the presence or absence of rs4680, rs4818, rs6269, and/or rs4633 in a COMT gene. Non-limiting examples of methods that can be used to detect the presence or absence of a COMT polymorphism, COMT haplotype and/or COMT genotype profile include a polymerase chain reaction (PCR) analysis, sequencing analysis (e.g., next generation sequencing), electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, Hpall tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfate-driven conversion of non-methylated cytosine to uracil, methyl-binding PCR analysis, the like or combinations thereof.

As used herein, the term “sequencing” is used in a broad sense and refers to any technique known in the art that allows the order of at least some consecutive nucleotides in at least part of a nucleic acid to be identified, including without limitation at least part of an extension product or a vector insert. Non-limiting examples of sequencing techniques include direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD® sequencing, MS-PET sequencing, mass spectrometry, and a combination thereof. In some embodiments, sequencing comprises an detecting the sequencing product using an instrument, for example but not limited to an ABI PRISM® 377 DNA Sequencer, an ABI PRISM® 310, 3100, 3100-Avant, 3730, or 373Oxl Genetic Analyzer, an ABI PRISM.® 3700 DNA Analyzer, or an Applied Biosystems SOLiD™ System (all from Applied Biosystems), a Genome Sequencer 20 System (Roche Applied Science), or a mass spectrometer. In certain embodiments, sequencing comprises emulsion PCR. In certain embodiments, sequencing comprises a high throughput sequencing technique, for example but not limited to, massively parallel signature sequencing (MPSS).

In certain embodiments, methods of this invention further comprise comparing the identified difference of the disease or condition-specific markers to a repository of at least one markers known in the art. Such comparison can further confirm the presence of the disease or condition. In some embodiments, the repository of the known markers can be obtained by data mining. The term “data mining”, as used herein, refers to a process of finding new data patterns, relations, or correlations derived from the known data of the databases and of extracting practicable information in the future. Typically a computer-based system can be trained on data to perform the data mining, e.g., to classify the input data and then subsequently used with new input data to make decisions based on the training data. These systems include, but are not limited, expert systems, fuzzy logic, non-linear regression analysis, multivariate analysis, decision tree classifiers, and Bayesian belief networks.

In further embodiments of the invention, a protein profile can be a protein expression profile, a protein activation profile, or a combination thereof. In some embodiments, a protein activation profile can comprise determining a phosphorylation state, a glycosylation state, methylation state, lipid modification, an ubiquitination state, a myristoylation state, conformational change in protein folding, monomerization and/or polymerization, and a conformational state of the protein.

A protein profile can be detected by any methods known in the art for detecting protein expression levels, protein phosphorylation state, protein ubiquitination state, protein myristoylation state, or protein conformational state. In some embodiments, a protein profile can be determined by an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APP I-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassay, microscopy, microfluidic chip-based assays, surface plasmon resonance, sequencing, Western blotting assay, or a combination thereof.

### Pharmaceutical Applications

As used herein, “administering” or “administration of” a compound or an agent to a subject with a particular COMT genetic profile can be carried out using one of a variety of methods known to those skilled in the art. For example, a compound or an agent can be administered, intravenously, arterially, intradermally, intramuscularly, intraperitonealy, intravenously, subcutaneously, ocularly, sublingually, orally (by ingestion), intranasal (by inhalation), intraspinally, intracerebrally, rectally, vaginally, and transdermally (by absorbtion, e.g., through a skin duct). A compound or agent can also appropriately be introduced by rechargeable or biodegradable polymeric devices or other devices, e.g., patches and pumps, or formulations, which provide for the extended, slow, or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. In some embodiments, a compound or an agent is administered orally, e.g., to a subject by ingestion, or intravenously, e.g., to a subject by injection. In some embodiments, the orally administered compound or agent is in an extended release or slow release formulation, or administered using a device for such slow or extended release.

All publications, patents and patent applications cited herein, whether supra or infra, are hereby incorporated by reference in their entirety. As used in this specification and the appended claims, the singular forms “a,” “an,” and “the” include plural references unless the content clearly dictates otherwise. The terms used in this invention adhere to standard definitions generally accepted by those having ordinary skill in the art. In case any further explanation might be needed, some terms have been further elucidated below.

### Representative Sequences

Shown below is a representative nucleic acid sequence of mRNA transcript variant X1 of Homo sapiens catechol-O-methyltransferase (COMT)(NCBI Reference Sequence: XM_{—}005261229.1). This sequence was derived from genomic sequence NT 011520.13 annotated using a gene prediction method. The coding sequence is shown from nucleotide 502 to nucleotide 1317.

The location of the rs4680 polymorphism is at nucleotide number 973 where the G allele is shown. The location of the rs4633 polymorphism is at nucleotide number 687 where the C allele is shown. The location of the rs6269 polymorphism is at nucleotide 404 where the A allele is shown. The location of the rs4818 polymorphism is at nucleotide 909 where the C allele is shown.

The location of additional polymorphisms listed in Table 1 are described in NCBI Reference Sequence: XM_{—}005261229.1.

| COMT mRNA transcript variant X1 | |
|---|---|
| NCBI Reference Sequence: XM_005261229.1 | |
| LOCUS: XM_005261229 | |
| (SEQ. ID. NO: 76) | |
| 1 | gcgccaccgc cattgccgcc atcgtcgtgg ggcttctggg gcagctaggg ctgcccgccg |
| 61 | cgctgcctgc gccggaccgg ggcgggtcca gtcccgggcg ggccgtcgcg ggagagaaat |
| 121 | aacatctgct ttgctgccga gctcagagga gaccccagac ccctcccgca gccagagggc |
| 181 | tggagcctgc tcagaggtgc tttgaagagg atccctgggc tgcctgggga agcacccagg |
| 241 | gccagggagt gtgaccctgc aggctccaca caggactgcc agaggcacac acctgctctg |
| 301 | tctacccgag ggcaccagag ggcacgagaa ggctggctcc ctggcgctga cacgtcaggc |
| 361 | aactgaggca caaggctggc atttctgaac cttgcccctc tgcaaacaca agggggcgat |
| 421 | ggtggcactc caagcaaagg ggcgtgtggg tgctgcagga ggagcacaga gcactggcgc |
| 481 | ccctcccctc ccgccctgca gatgccggag gccccgcctc tgctgttggc agctgtgttg |
| 541 | ctgggcctgg tgctgctggt ggtgctgctg ctgcttctga ggcactgggg ctggggcctg |
| 601 | tgccttatcg gctggaacga gttcatcctg cagcccatcc acaacctgct catgggtgac |
| 661 | accaaggagc agcgcatcct gaaccacgtg ctgcagcatg cggagcccgg gaacgcacag |
| 721 | agcgtgctgg aggccattga cacctactgc gagcagaagg agtgggccat gaacgtgggc |
| 781 | gacaagaaag gcaagatcgt ggacgccgtg attcaggagc accagccctc cgtgctgctg |
| 841 | gagctggggg cctactgtgg ctactcagct gtgcgcatgg cccgcctgct gtcaccaggg |
| 901 | gcgaggctca tcaccatcga gatcaacccc gactgtgccg ccatcaccca gcggatggtg |
| 961 | gatttcgctg gcgtgaagga caaggtcacc cttgtggttg gagcgtccca ggacatcatc |
| 1021 | ccccagctga agaagaagta tgatgtggac acactggaca tggtcttcct cgaccactgg |
| 1081 | aaggaccggt acctgccgga cacgcttctc ttggaggaat gtggcctgct gcggaagggg |
| 1141 | acagtgctac tggctgacaa cgtgatctgc ccaggtgcgc cagacttcct agcacacgtg |
| 1201 | cgcgggagca gctgctttga gtgcacacac taccaatcgt tcctggaata cagggaggtg |
| 1261 | gtggacggcc tggagaaggc catctacaag ggcccaggca gcgaagcagg gccctgactg |
| 1321 | cccccccggc ccccctctcg ggctctctca cccagcctgg tactgaaggt gccagacgtg |
| 1381 | ctcctgctga ccttctgcgg ctccgggctg tgtcctaaat gcaaagcaca cctcggccga |
| 1441 | ggcctgcgcc ctgacatgct aacctctctg aactgcaaca ctggattgtt cttttttaag |
| 1501 | actcaatcat gacttcttta ctaacactgg ctagctatat tatcttatat actaatatca |
| 1561 | tgttttaaaa atataaaata gaaattaaga atctaaatat ttagatataa |

Shown below is a representative polypeptide sequence of Homo sapiens catechol-O-methyltransferase (COMT) as translated from NCBI Reference Sequence XM_{—}005261229.1. The amino acid sequence is similar to UniProt P21964. Amino acids 1 to 271 are shown.

The location of the rs4680 polymorphism is at amino acid number 158 where the Val158 allele is shown. The location of the rs6267 polymorphism is at amino acid number 72 where the Alanine allele is shown. The presence of the rs6267 polymorphism results in a substitution of Alanine at position 72 with Serine at position 72. The Serine 72 variant is correlated with reduced COMT enzyme activity. Amino acids 1-50 are missing in the soluble form of this protein. The position of additional polymorphisms (amino acid substitutions) can be found online at UniProt (URL: http://www.uniprot.org/uniprot/P21964, accessed Oct. 13, 2014). polymorphisms listed in Table 1 are described in NCBI Reference Sequence: XM_{—}005261229.1.

COMT Polypeptide

(SEQ. ID. NO: 77)

MPEAPPLLLA AVLLGLVLLV VLLLLLRHWG WGLCLIGWNE
FILQPIHNLL MGDTKEQRIL NHVLQHAEPG NAQSVLEAID
TYCEQKEWAM NVGDKKGKIV DAVIQEHQPS VLLELGAYCG
YSAVRMARLL SPGARLITIE INPDCAAITQ RMVDFAGVKD
KVTLVVGASQ DIIPQLKKKY DVDTLDMVFL DHWKDRYLPD
TLLLEECGLL RKGTVLLADN VICPGAPDFL AHVRGSSCFE
CTHYQSFLEY REVVDGLEKA IYKGPGSEAG P*

### EXAMPLES

### Example 1

### Irritable Bowel Syndrome

### Materials and Methods

A randomized clinical trial investigating placebo effects in IBS patients (Trial registration—NCT00065403) was conducted. Details of the design and outcomes of the trial are provided elsewhere. The 3-week trial enrolled 262 patients (75% women) ≧18 years and diagnosed by IBS Rome II criteria score of >150 on the Irritable Bowel Syndrome Symptom Severity Scale (IBS-SSS).

Patients were randomized to one of three treatment arms: (1) no-treatment control (“waitlist); (2) placebo acupuncture (“limited”); (3) placebo acupuncture plus a supportive patient-provider (“augmented”). A validated sham acupuncture device was used to deliver placebo acupuncture in 20 minute sessions, twice weekly for three weeks.

A subgroup of patients (n=112) gave consent for genetic analysis from blood samples included in this study. The Institutional Review Board at Beth Israel Deaconess Medical Center (Boston, Mass.) approved the main study and the genetic follow-up study presented here. All studies were conducted in accordance with the Declaration of Helsinki. Participants provided written consent for this genetic study. The ethics committee approved this procedure.

Three validated IBS research measures from the previous trial, which assessed clinical outcomes, were used in this study. The primary outcome measure was the IBS-SSS, which consists of five 100-point scales (abdominal pain severity, abdominal pain frequency, abdominal distention severity, dissatisfaction with bowel habits, and quality of life disruption) that contribute equally to the final score, yielding a theoretical range of 0-500. Higher scores reflected a more severe condition. IBS-SSS was measured at baseline and after 3 weeks of treatment. Change in IBS-SSS was determined by subtracting 3-week IBS-SSS score from baseline IBS-SSS. IBS-SSS was selected as a primary outcome because the two secondary measures, Adequate Relief (AR) and the Global Improvement Scale (GIS) did not have baseline measures. Adequate Relief was assessed by a single dichotomous question at 3-weeks, which asked: “Over the past week have you had adequate relief of your IBS symptoms?”. The GIS asked: “Compared to the way you felt before you entered the study, have your IBS symptoms over the past 7 days been: (1)=substantially worse, (2)=moderately worse, (3)=slightly worse, (4)=no change, (5)=slightly improved, (6)=moderately improved, or (7)=substantially improved”. These latter two measures were considered secondary because they do not have baseline assessments, thus opening the door to regression artifacts. To mitigate this problem, IBS-SSS baseline scores were controlled in analyses of AR and GIS.

Of the 262 original study participants, 112 gave consent for genetic screening. Eight patients missing IBS-SSS data at 3-weeks were excluded from the analyses. Two additional patients were missing data for AR and GIS and were excluded from analysis of AR and GIS. Genomic DNA was extracted from whole blood using Qiagen Blood kit (Valencia, Calif.) following the manufacturer's protocol. Based on the association of COMT SNP rs4633 with COMT expression and activity, this SNP was genotyped in addition to rs4680 (Val158met). TaqMan SNP Genotyping assays for rs4680 (Val158met) and rs4633 were purchased from Applied Biosystems, (Foster City, Calif.). Quantitative PCR was performed at the Biopolymers Facility at Harvard Medical School, (Boston, Mass.) following the manufacturer's protocol on an Applied Biosystems 7900HT instrument, using SDS version 2.4 software.

Hardy-Weinberg Equilibrium (HWE) and Linkage Disequilibrium were calculated using the Online Encyclopedia for Genetic Epidemiology studies. Statistical analyses were performed using IBM SPSS Statistics version 20 (Chicago, Ill.).

An additive genetic model was used to investigate the linear effect of increases in the presence of the COMT met allele. A variable was created, “COMT genotype”, that coded each patient's Val158met genotype as follows: 1=Met/Met; 0=Val/Met; −1=Val/Val. Using standard coding for polynomial trends, multiple regression was used to examine linear and quadratic effects of COMT genotype (number of met alleles) on placebo responses as measured by changes from baseline IBS-SSS, and on AR and GIS. Initial disease severity was controlled for by including baseline IBS-SSS as a covariate in the regression models. In addition, variables were created to test for linear and quadratic effects of treatment, conceiving these as ascending “doses” of non-specific effects (waitlist, limited placebo, augmented placebo) to test for interactions between COMT genotype effects and treatment received.

The clinical and demographic characteristics of the subset of genotyped patients (n=104) relative to the original clinical trial (n=262) are shown in Table 2. Age, gender, race and marital status of the genotyped patients did not differ significantly from the distribution in the original study; duration, IBS type and baseline IBS-SSS were also similar. Eighty percent of the genotyped patients were women and 94% were white. Furthermore there were no significant differences in demographics and disease characteristics of genotyped patients across the COMT Val158met genotypes. The number of patients genotyped and analyzed from each treatment arm (waitlist, 29%; limited, 32%; and augmented, 39%) was similar to the overall distribution in the original trial (waitlist, 33%; limited, 34%; and augmented, 33%).

TABLE 2
Baseline characteristics of genotyped IBS patients compared
to participants in the parent randomized clinical trial

| | All IBS | Genotyped | | | | | |
|---|---|---|---|---|---|---|---|
| | patients* | patients | p | COMT rs4680 Genotype | | | p |
| Characteristics | (n = 262) | (n = 104) | value | Met/Met | Met/Val | Val/Val | value |

Demographics

| | | | | | | | |
|---|---|---|---|---|---|---|---|
| Age in years (SD) | 38 (14) | 37 (13) | 0.53 | 40 (12) | 36 (13) | 38 (14) | 0.44 |
| Women (%) | 218 (75) | 83 (80) | 0.51 | 16 (80) | 45 (81) | 22 (76) | 0.82 |
| White (%) | 252 (88) | 98 (94) | 0.17 | 20 (100) | 52 (95) | 26 (89) | 0.20 |
| Married/living | 101 (45) | 32 (39) | 0.42 | 8 (42) | 16 (41) | 8 (33) | 0.79 |
| together (%) | | | | | | | |

IBS symptoms and baseline characteristics

| | | | | | | | |
|---|---|---|---|---|---|---|---|
| Type of IBS | | | 0.56 | | | | 0.80 |
| Constipation (%) | 74 (27) | 35 (34) | | 5 (25) | 21 (39) | 9 (31) | |
| Diarrhea (%) | 65 (23) | 18 (17) | | 4 (20) | 8 (15) | 6 (21) | |
| Mixed (%) | 139 (50) | 40 (49) | | 11 (55) | 25 (46) | 14 (48) | |
| Disease for | 103 (35) | 63 (61) | 0.18 | 12 (60) | 34 (62) | 17 (59) | 0.96 |
| >5 years (%) | | | | | | | |
| Baseline IBS-SSS | 273 (73) | 270 (66) | 0.72 | 291 (78) | 261 (64) | 272 (61) | 0.23 |
| (SD) | | | | | | | |

Based on its association with rs4680, COMT expression and enzymatic activity rs4633 was also genotyped. Rs4633 was found to be in strong linkage disequilibrium with rs4680 (D′=0.94 and r2=0.9), such that the two SNPs were almost perfectly correlated: Met/Met, Val/Met and Val/Val of rs4680 corresponded to the T/T, T/C and C/C of rs4633. The minor allele frequency of COMT Val158met was 0.46 and the SNP was in Hardy-Weinberg Equilibrium (p=0. 502). See Table 3.

TABLE 3
Characterization of Selected Polymorphisms

| SNP | Alleles | Minor Allele | MAF | Mutation Type | HWE p |
|---|---|---|---|---|---|
| rs4680 | A/G | A | 0.46 | Mis-sense Mutation | >.05 |
| | | | | Transition Substitution | |
| rs4633 | C/T | T | 0.47 | Silent Mutation | >.05 |
| | | | | Transition Substitution | |
| rs4818 | G/C | G | 0.44 | Transversion | >.05 |
| | | | | Substitution | |

In this study, IBS Symptom Severity Scale (IBS-SSS) was a priori primary clinical outcome. IBS-SSS is a multidimensional measure that captures the full spectrum of IBS disease including abdominal pain severity, abdominal pain frequency, abdominal distention severity, dissatisfaction with bowel habits, and disruption in quality of life and has a theoretical range of 0-500.

Both linear and quadratic effects of COMT alleles and treatment arm (waitlist, limited, augmented) were tested. As there were no significant main effects or interactions involving quadratic tests, results reported here are for a trimmed model with only linear effects and interactions included.

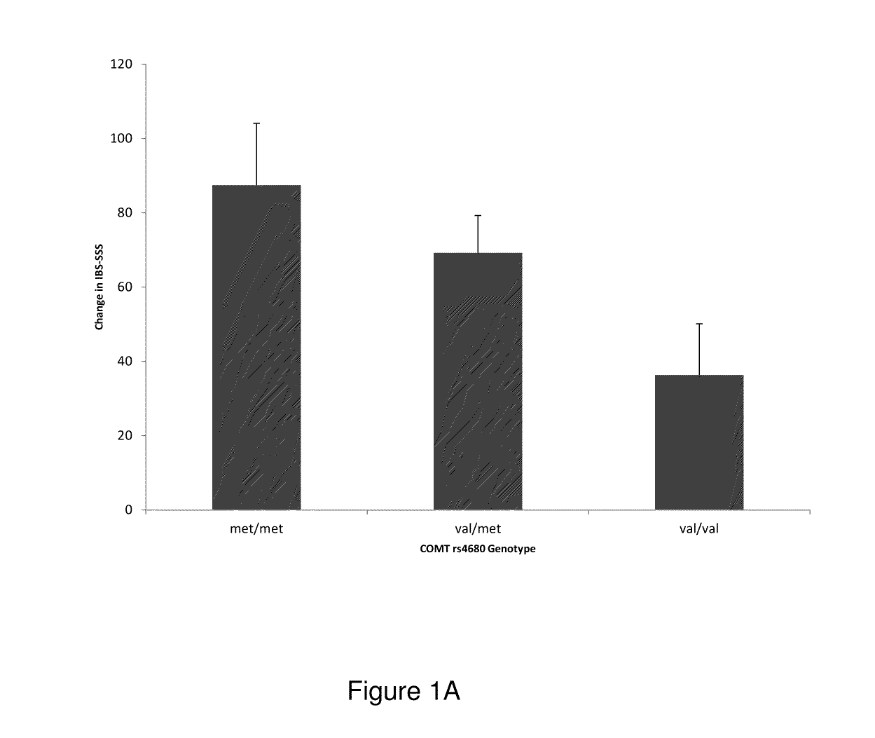

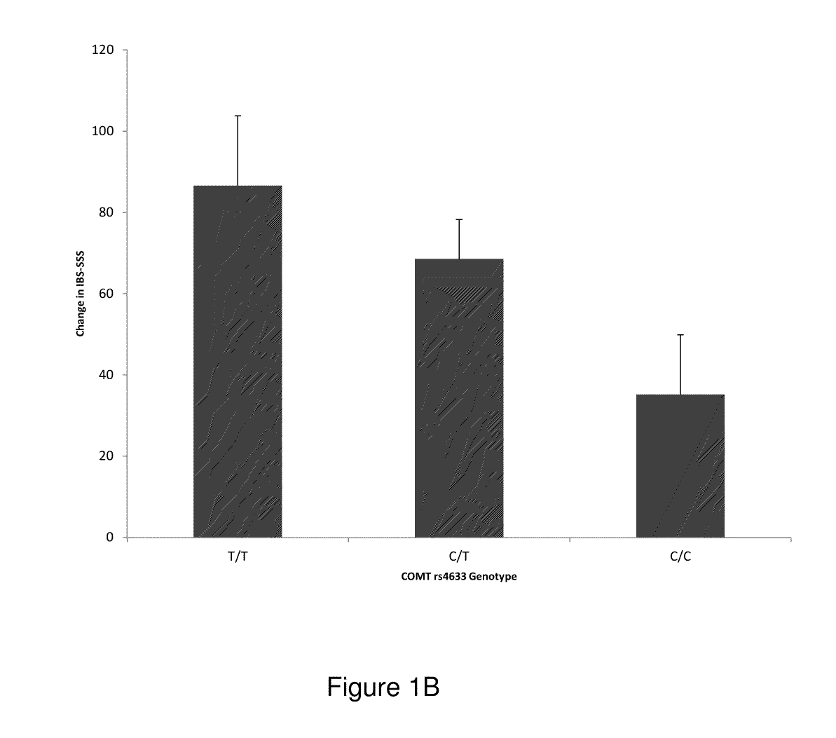

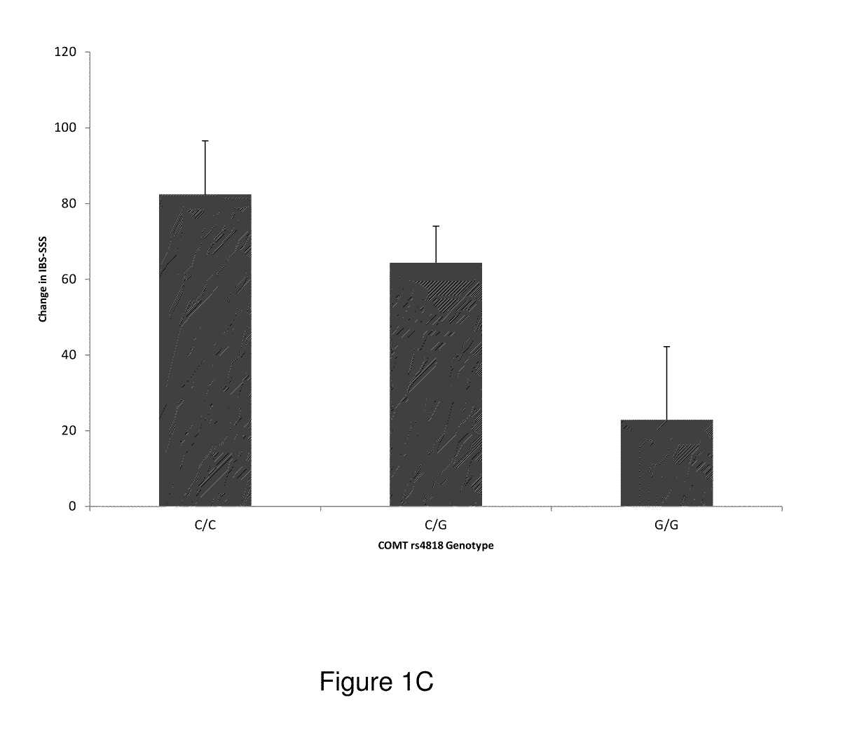

Change in IBS-SSS was associated with a main effect of COMT genotype (number of met alleles) (FIGS. 1A-1C). Number of COMT val158met met alleles showed a significant linear effect on IBS-SSS (beta=0.17; p=0.032). Patients with the Met/Met genotype had the greatest level of improvement whereas Val/Val patients had the least, and Val/Met patients were intermediate (FIG. 1A). As in the original study, there was a main effect of treatment arm on improvement, with patients in the augmented treatment improving most and those on waitlist improving least (beta=0.19, p =0.019).

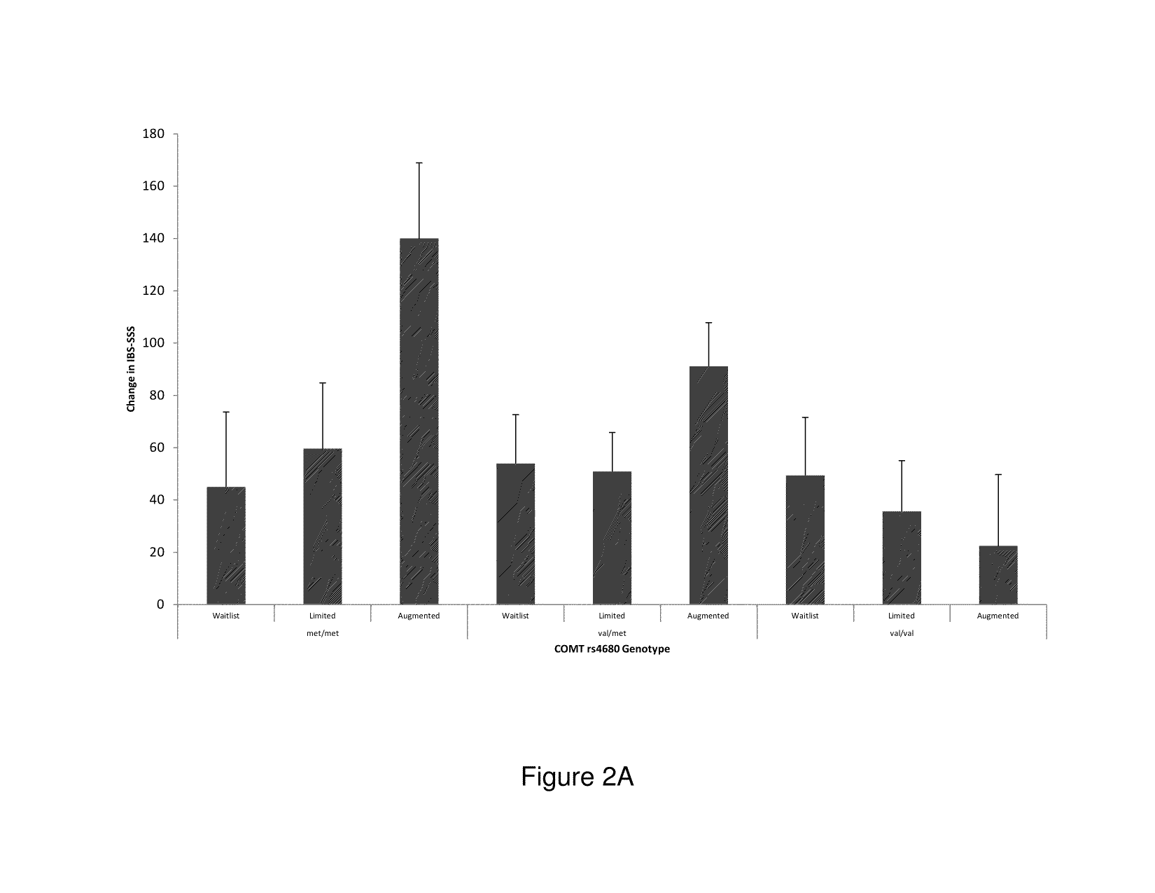

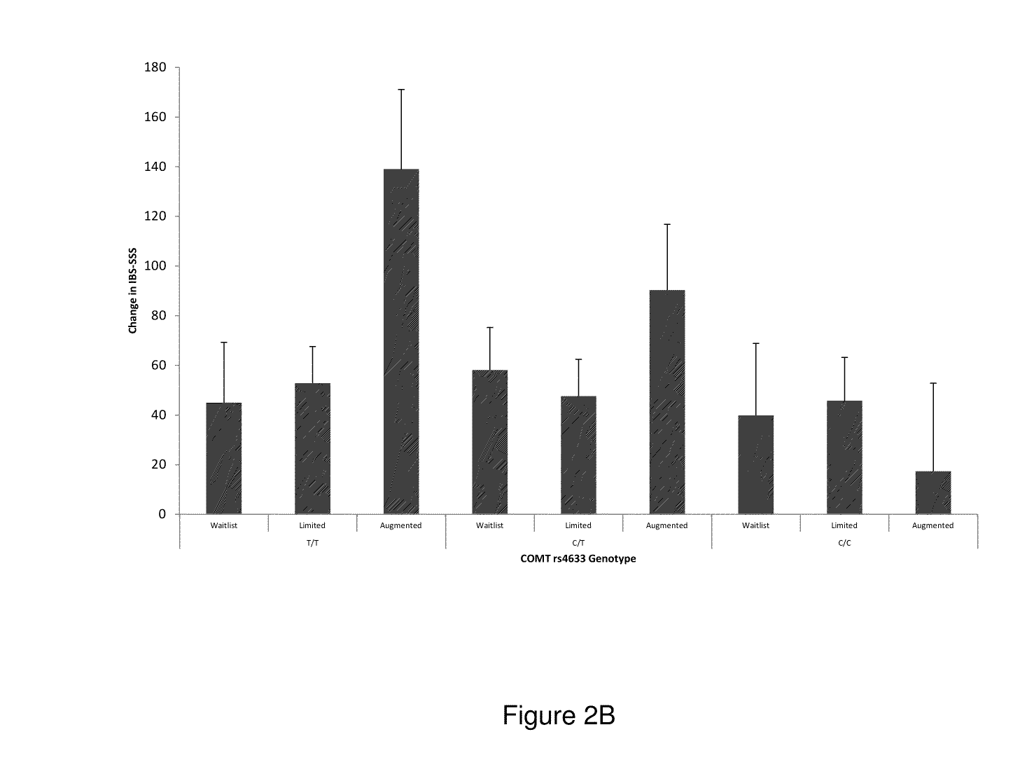

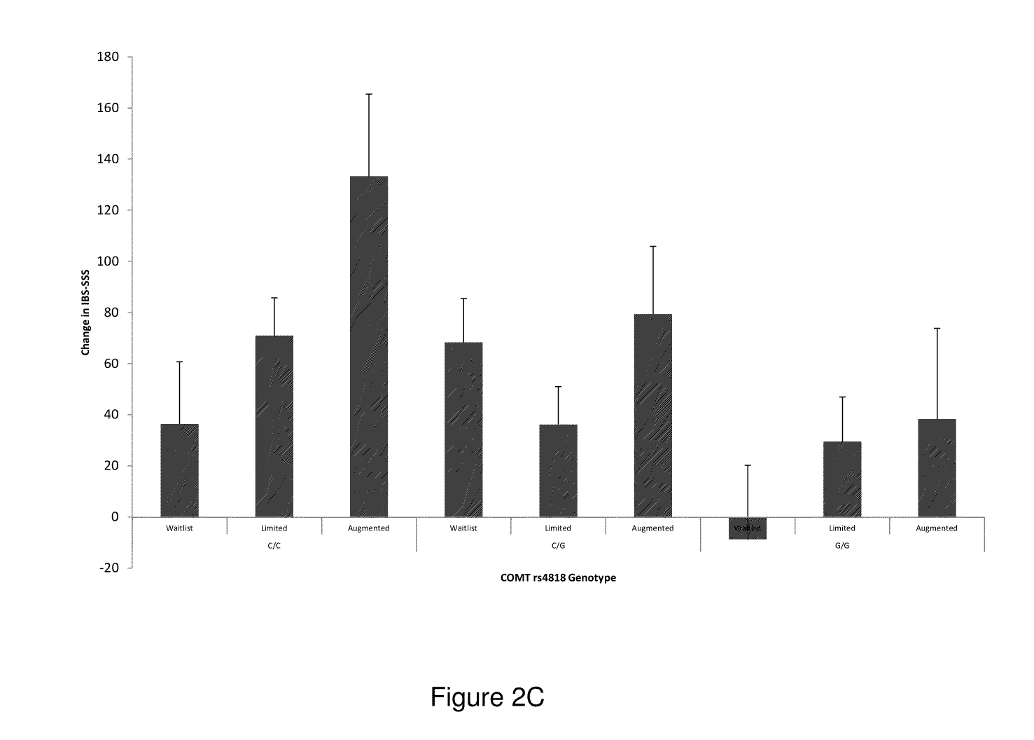

These linear effects on IBS-SSS were qualified by significant interactions between COMT genotype and treatment arm (beta=0.17; p=0.035) (FIGS. 2A-2C). COMT predicted responses in only the augmented placebo arm. In response to a high dose placebo treatment, patients homozygous for met alleles (Met/Met), showed the greatest improvement; Val homozygotes (Val/Val) showed the least improvement and heterozygote (Val/Met) patients had an intermediate response (FIG. 2A).

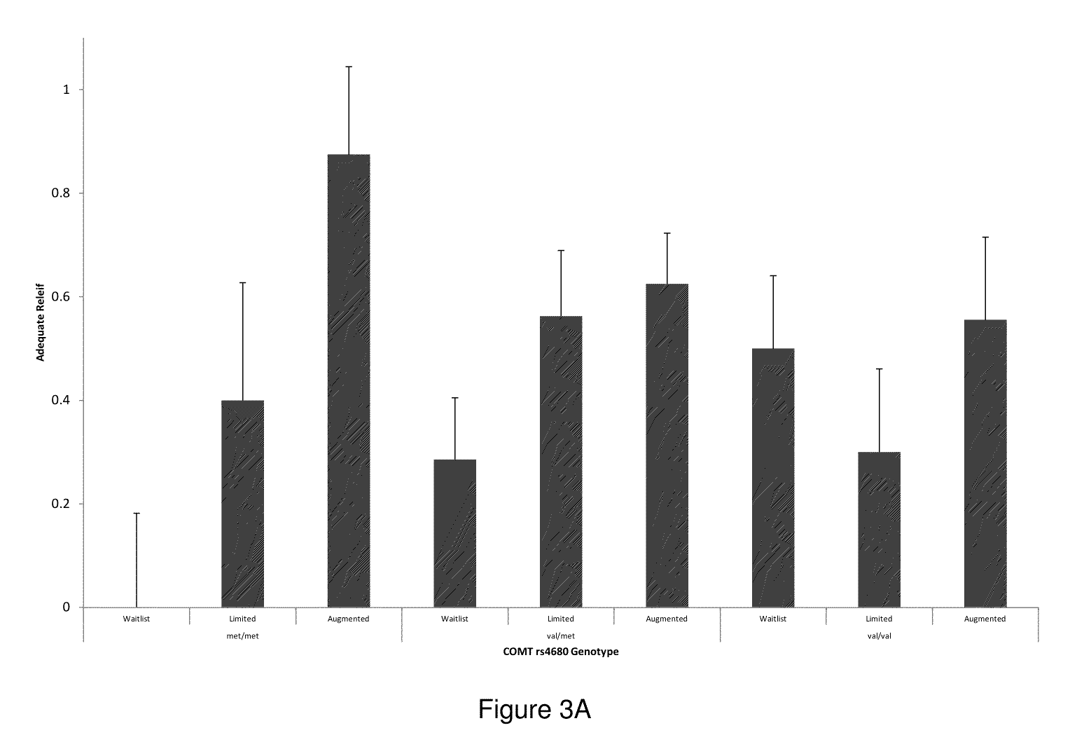

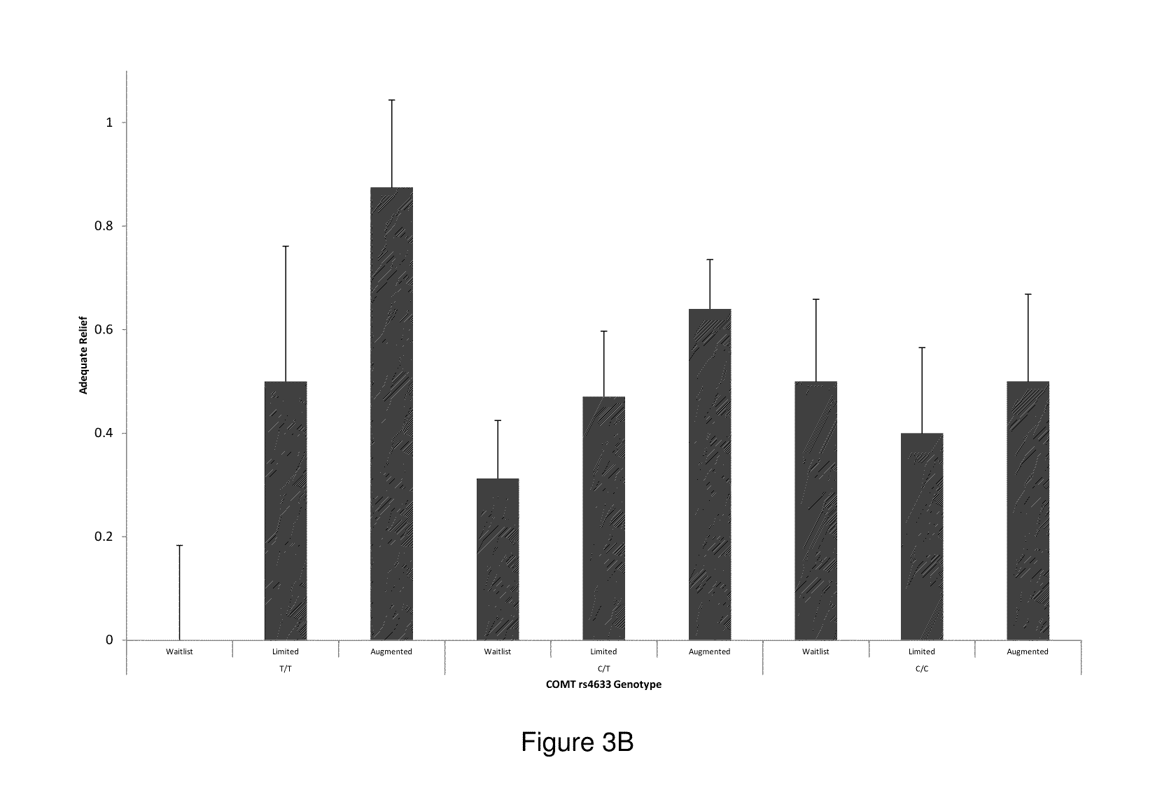

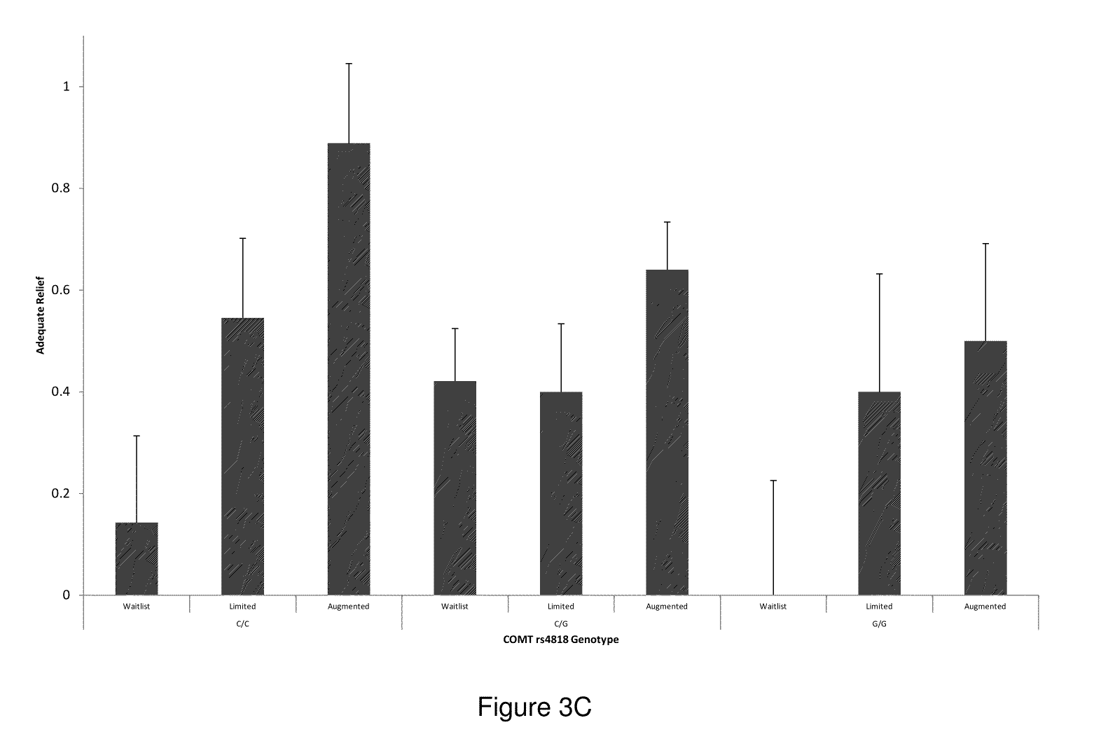

The secondary validated measure of Adequate Relief uses a single dichotomous question at 3-weeks which asked: “Over the past week have you had adequate relief of your IBS symptoms?”. The responses were coded as 0=No, I did not have Adequate Relief and 1=Yes, I had Adequate Relief. As expected from the previous study, there was a significant linear effect for treatment arm (beta=0.32, p=0.001) (FIGS. 3A-3C), in which patients in the augmented placebo arm showed most improvement and those in the waitlist arm showed the least. Although there was no main effect for COMT genotype, there was a significant interaction between COMT genotype and treatment arm (beta=0.25, p=0.009) (FIGS. 3A-3C). In the augmented placebo arm, the Met/Met patients reported an average Adequate Relief score of 0.88 compared to 0.63 for Val/Met patients and 0.56 for Val/Val patients (FIG. 3A). Conversely, in the waitlist group, Val/Val patients had an average score of 0.50, whereas the Met/Met patients had an average score of 0.00, that is no Met/Met patients on the waitlist arm reported Adequate Relief.

The other secondary validated measure was Global Improvement Scale (GIS) which asked: “Compared to the way you felt before you entered the study, have your IBS symptoms over the past 7 days been: (1)=substantially worse, (2)=moderately worse, (3)=slightly worse, (4)=no change, (5)=slightly improved, (6)=moderately improved, or (7) =substantially improved”. As in the case of IBS-SSS and Adequate Relief, there was an expected significant linear effect for treatment arm (beta=0.31, p=0.002), in which patients in the augmented placebo arm showed most improvement and those in the waitlist arm showed the least. There was a trend in improvement associated with COMT genotype (beta=0.18, p=0.063) but no interaction of COMT genotype with treatment arm (beta=0.07, p=0.477).

### Example 2

### Association of COMT Genotype with a Placebo Response to Pain

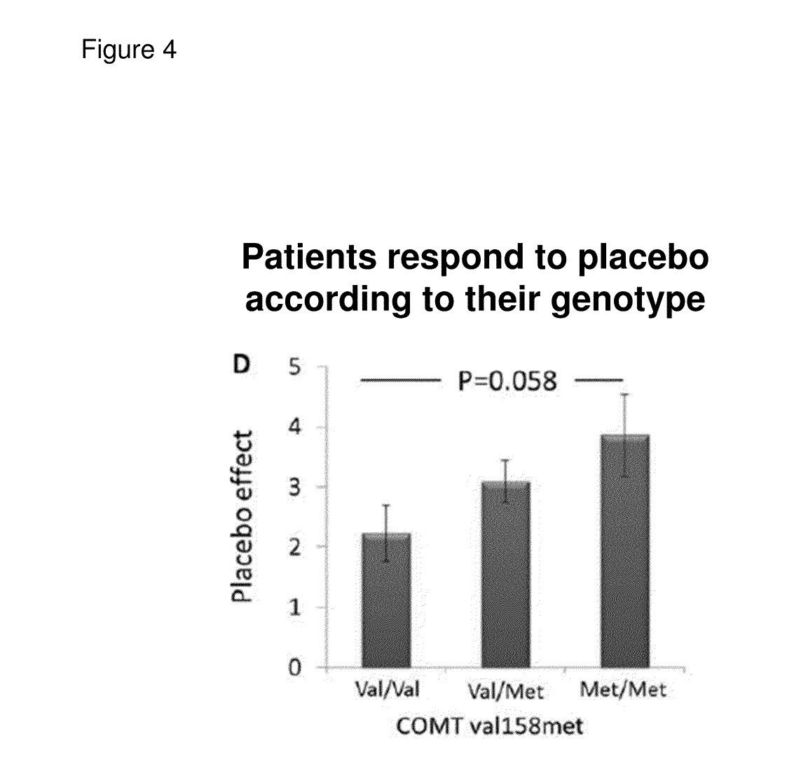

FIG. 4 is a bar graph showing the response of subjects to a pain stimulus in the presence of an analgesic placebo cue. Subjects were exposed to low or high, local heat stimulus on their forearm. During a conditioning phase the subjects learned to distinguish between a high heat and a low heat pain stimulus which was accompanied by a cue card describing the pain sensation that should be expected as “low” or “high”. In the next phase, subjects received low or high pain stimuli. In a randomized manner some of the high pain administrations were accompanied by a cue card predicting a “low” pain stimulus. This is the equivalent of a placebo analgesic. The bar graph shows the correlation of a positive response to this placebo analgesic correlated to the rs4818 COMT genotypes of the tested subjects. The ρ value in the 46 subjects tested is 0.058.

### Example 3

### Clinical Trial with Placebo Prescreen

Thase and colleagues (Arch Gen Psychiatry, vol 53, pp. 777-784, 1996) found that sertraline produced a clinical improvement in 59% of the 416 dysthymic patients and that the corresponding placebo treatment produced improvement in 44% of the 416 patients. The required numbers of patients for statistically significant demonstration of efficacy over placebo can be largely reduced, compared to non-prescreened trials. Prescreening patients for Met/Met and their subsequent exclusion from the clinical trial can result in placebo reduction. Using Fisher's exact test (two-tailed with α=5%), a total of 366 patients would be needed to have 80% power to detect a significant effect of medication over placebo. This example is based on the Thase sertraline (Zoloft) study. However, by identifying likely placebo responders and reducing the proportion of placebo responders to 24%, a study of only 72 patients would be needed to achieve 80% power. By identifying likely placebo responders and reducing the proportion of placebo responders to 34%, a study of only 140 patients would be needed to achieve 80% power.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### Example 4

### Prophetic Example—Altering or Modulating a Treatment

A patient is treated for a symptom with a commercially available treatment (e.g. drug, device etc.). A medical professional tests the patient for one or more specific COMT polymorphism genotypes. The medical professional detects the presence of a COMT polymorphism genotype that indicates the patient is likely or highly likely to exhibit a placebo response if administered a placebo treatment. For example the patient tests positive for a Met158 allele and is heterozygous (e.g., Val/Met) or homozygous (e.g., Met/Met) for the polymorphism. If the patient is of high predisposition for a placebo response genotype, then the medical professional administers an additional non-pharmacological or non-device placebo treatment to the commercially available treatment response. Such “placebo” treatment could include: improvement of the therapeutic encounter (e.g., spend more “quality” time with patient (e.g., this was done during the IBS study in the group called “augmented”)), administer a placebo pill, administer non-traditional treatments for which no evidence of pharmacological efficacy is provided such as massage therapy, acupuncture, relaxation therapy and the like. Lets call all these additional options “placebo”. The formula for a response is then:

COMT placebo responder haplotype (most likely MM)+treatment (drug or device)+placebo>COMT placebo responder haplotype+treatment, or COMT non-placebo haplotype+treatment.

In addition, when the patient has an augmented treatment response from treatment+placebo, the doctor may choose to taper the drug/device dose in order to reduce side-effects.

### Example 5

### Placebo-Associated Polymorphisms

### Biomarkers of the Placebo Response

### Towards a Physiology of the Placebo Response

A growing list of neurotransmitters and neurological pathways mediating the placebo response provide a framework for candidate gene analyses. Indeed, treatment outcomes in the placebo arms of trials that have assessed genetic variation in the dopaminergic, opioid, cannabinoid, and serotonergic pathways suggest that genetic variation in the synthesis, signaling, and metabolism of these neurotransmitters may contribute to variation in the placebo response (Table 4).

### Genetic Variation in the Dopamine Pathway

The emergence of dopamine-mediated reward centers as central to the underlying physiology of the placebo response make genetic variation in dopamine metabolism and signaling pathway genes prime candidates for placebo response biomarkers. Catechol-O-methyltransferase (COMT) is an enzyme that metabolizes dopamine and other catecholamines. The rs4680 single nucleotide polymorphism (SNP) is in the COMT gene. Rs4680 encodes a valine (val)-to-methionine (met) change at codon 158 (val158met) resulting in a three-to four fold reduction in enzymatic activity. Homozygotes of the less-active met allele are associated with higher levels of dopamine in the pre-frontal cortex, a region implicated in the placebo response pathway. The prevalence of the less frequent met allele or minor allele frequency (MAF) is reported as 0.37 in Caucasians, but varies by race/ethnicity. The MAF of rs4680 translates to an estimated 20-25% of met/met individuals in Caucasian populations.

One candidate genetic association study that included a no treatment control (NTC) and examined the effect of genetic variation in COMT on the placebo response used an RCT designed to test whether placebo treatment could incrementally combine three components related to placebos: diagnosis and observation (NTC arm), therapeutic apparatus (placebo acupuncture), and apparatus plus a supportive patient-practitioner relationship (placebo acupuncture plus a warm-caring provider). The RCT was a three-week trial in patients with irritable bowel syndrome (IBS), and the main outcome was reduction in IBS symptom severity. Patients in the arm that combined all the components, the strongest placebo treatment, reported the greatest symptom relief. The candidate genetic analysis performed on a subset of these patients, who gave genetic informed consent, looked at the association of rs4680 with IBS symptom severity, adequate relief, and quality of life in each of the treatment arms. Patients homozygous for the rs4680 low-activity met allele (met/met), known to have the high levels of dopamine, had the greatest placebo response. The high-activity val allele homozygous (val/val) patients had the lowest placebo response. The val/met heterozygotes had an intermediate response. Similar results were reported for another COMT SNP, rs4633, which is closely linked to rs4680.

A subsequent small acute-pain model placebo neuroimaging study in healthy volunteers looked at genetic variation in COMT in relation to brain activity in the reward system using resting-state functional magnetic resonance imaging. These researchers showed that placebo response to pain in healthy volunteers supported the IBS results such that the number of rs4680 met alleles was linearly correlated with suppression of pain in the placebo expectation laboratory paradigm. While not having a NTC, the pain stimulation in this experiment was momentary, precise, and calibrated, so one can assume that spontaneous remission and waxing and waning of illness are not potential confounders.

Interestingly, a recent laboratory study found that the rs4680 high-activity val allele was associated with a higher frequency of nocebo effects (negative placebo side-effect) using a model of learned immunosuppression. Similarly, in the IBS placebo study discussed previously, the rs4680 high-activity val allele was associated with a higher frequency of complaint reporting. This association of nocebo effect with the high-activity rs4680 val allele is not necessarily unexpected given that in the absence of any significant improvements in symptoms derived from a placebo response, val/val individuals may have more complaints or experience more side-effects.

In addition to COMT there are several other polymorphisms in the dopamine pathway that are potential PAP candidates. Monoamine oxidase A (MAO-A) has been implicated in reward pathways through its role in catalyzing the oxidation of monoamines including dopamine. MAO-A also metabolizes serotonin and has been shown to affect serotonergic availability and signaling. The MAO-A gene is X-linked, and a common rs6323 (G to T) SNP results in a 75% reduction in enzymatic activity in females homozygous for the T allele, and males hemizygous with one T allele. The association of MAO-A with treatment response to placebo was examined in a candidate gene analysis of patients with clinical depression from four combined small placebo-controlled RCT's of three selective serotonin reuptake inhibitor antidepressants (SSRIs), venlaxafine, sertraline, or fluoxetine. The primary outcome was determined by the 17-item Hamilton Depression Rating Scale (HAM-D17). Consistent with the findings described above for COMT, individuals with the low-activity MAO-A genotypes and, therefore, higher basal dopamine tone had a higher placebo response than those with the high-activity MAO-A genotypes. The COMT rs4680 association with placebo response was also examined in this study, but the results were not significant. It is unclear whether the non-significant results with COMT were due to lack of power, a basic difference in the subject population, or other factors.

One of the largestest studies of genetic variation in RCT patients randomized to placebo treatment examined 34 candidate genes (500 polymorphisms) in four trials of bupropion for major depressive disorder. Although results for rs4680 were not reported in this trial, several other COMT SNPs were associated with placebo response and placebo remission (although these associations did not survive correction for multiple comparisons). The placebo response association with MAO-A rs6609257, a SNP associated with dopamine basal tone, was one of the associations with treatment response in the placebo arm that was significant after correction, supporting the candidacy of MAO-A as a PAP.

Genetic variations in dopamine receptor genes which modify dopaminergic signaling also modify the function of the brain reward circuit. Rs6280 is a common serine-to-glycine coding polymorphism in dopamine receptor 3 (DRD3) that results in the DRD3 glycine form having a higher affinity for dopamine than the serine form. A recent placebo-controlled RCT of a novel drug for treating symptoms of schizophrenia (ABT-925) examined the effects of genetic variation in DRD3 on the Positive and Negative Syndrome Scale (PANSS). Subjects homozygous for rs6280 serine allele (S/S) had significantly better outcomes in the placebo arm than when they were treated with increasing doses of ABT-95. Consistent with other studies, this study also showed that the COMT rs4680 met/met subjects had a higher placebo response.

Genetic variation in dopamine beta-hydroxylase (DBH), an enzyme that converts dopamine to norepinephrine, like COMT, has been associated with variation in blood pressure and psychiatric disease. In studies of alcohol dependence, individuals homozygous for the CC genotype of the rs1611115 DBH polymorphism appeared to do better on placebo and worse on naltrexone. DBH was also one of the genes examined in the largest 54-candidate gene analysis of the placebo arm of the bupropion trial discussed above. The DBH SNP rs2873804 survived the correction for multiple comparisons reinforcing DBH as a potential candidate for a placebo response gene.

Brain-derived neurotrophic factor (BDNF) plays an important role in learning and memory, mediating and maintaining turnover of dopamine. BDNF's functions in neuroadaptive change and response to reward stimuli make it another plausible candidate for a PAP. The rs6265 SNP in BDNF encodes a valine-to-methionine substitution at codon 66. This functional polymorphism is hypothesized to reduce activity-dependent BDNF release due to inefficient BDNF trafficking to secretory granules. Genetic variation at rs6265 was associated with greater placebo-induced dopamine D2 and D3 activation in rs6265 val allele homozygotes compared to met allele carriers; however, these differences in neuronal activation did not translate into differences in placebo analgesia as assessed by the pain ratings reported.

These data show a consistent association of outcomes in patients and healthy volunteers treated with placebo with genes involved in dopamine metabolism and signaling such that individuals with higher levels of dopamine or higher dopaminergic activity tended to be more likely to respond to placebo in the studies examined. Taken together, these associations provide support for dopamine pathway SNPs as placebo response genetic markers. More research in other conditions, dopamine pathway SNPs, and with larger samples with NTCs would help to make these associations more definitive.

### Genetic Variation in the Opioid Signaling Pathway

Endogenous opioids signal through opioid receptors, and genetic variation in the p-opioid receptor gene (OPRM1) has been shown to modify treatment outcomes in pain studies. The analgesic effects of placebo have been shown to be mediated through activation of endogenous opioid as well dopaminergic mechanisms. In a small experimental placebo study performed on healthy volunteers, signaling in the dopamine pathway was linked to opioid receptor signaling in anti-nociceptive responses to placebo. (Nociceptive pain is thought to be caused by stimulation of pain receptors in response to pressure, temperature or irritating substances which send pain signals to the brain in response to injury or the possibility of injury. Anti-nociceptive treatments are designed to reduce such pain.)

Rs1799971 is a functional polymorphism in the OPRM1 gene that results in an asparagine-to-aspartic acid change at codon 40. The aspartic acid variant of the receptor was found to reduce receptor function across several studies. The association of rs1799971 with placebo response in healthy volunteers was studied in an experimental model of placebo-induced analgesia . In this study, placebo-induced activation of dopamine neurotransmission in the nucleus accumbens was greater in asparagine homozygotes compared to aspartic acid-allele carriers, suggesting that genetic variation in OPRM1 could also contribute to variability in the placebo response.

Whether or not the association of OPRM1 with placebo-induced analgesia is generalizable to other non-pain paradigms of placebo response remains to be determined. Indeed, work on genetic variation in OPRM1 has examined associations with the reward-based addictive effects of psychostimulants (e.g. amphetamine) and opioid drugs (e.g. morphine). Several of these studies have shown differential outcomes in the placebo and drug treatment arms as a function of genetic variation in OPRM1; but, again, it is impossible to determine if the effect modification of treatment outcomes in the placebo arm is a function of placebo response or genetic variation effects at baseline in the absence of a NTC.

### Genetic Variation in Endocannabinoids and Serotonin Signaling Pathways

The two other neurological pathways implicated in the placebo response involve endocannabinoid and serotonergic signaling. Endocannabinoids are neurotransmitters that signal through the cannabinoid receptors, CB1 and CB2, and have been implicated in analgesia. Placebo laboratory studies have further implicated endocannabinoids in placebo analgesia, providing a rational for considering genetic variation in the endocannabinoid pathway for identifying PAPs. The effects of genetic variation in fatty acid amide hydrolase (FAAH), the major degradative enzyme of endocannabinoids, was examined in a small study that used some of the same subjects as the OPRM1 placebo experiment described above . This study found that homozygotes for the FAAH Pro129 allele (known to increase chronically endocannabinoid levels in the brain in response to pain) reported more placebo-induced analgesia, supporting the endocannabinoid pathway genes as loci worth exploring for identifying PAPs.

Serotonin is a neurotransmitter that is important in regulating mood, appetite, and sleep. Given the high rates of placebo responses in RCTs of treatments for mood disorders, the serotonin pathway is important to examine for possible placebo response-related genes. SSRIs are antidepressants thought to block the uptake of serotonin. There is some evidence from candidate gene studies that serotonin pathway genes are associated with placebo responses of depression and anxiety. The previously mentioned study that examined 34 candidate genes for placebo response in depression included several genes in the serotonergic pathway and reported significant association between placebo remission with 5-hydroxytryptamine (serotonin) transporter SLC6A4 rs4251417, HTR2A rs2296972, and rs622337. Unfortunately, of the largest GWAS conducted to determine the effectiveness of different treatments for people with major depression, the Sequenced Treatment Alternatives to Relieve Depression (STAR*D) Study, did not include a placebo control.

Serotonin-mediated placebo response genes have also been examined in a small RCT of social anxiety disorder (SAD). In this small candidate gene PET study of SAD, reduction in anxiety symptoms in response to placebo was accompanied by a reduction in stress-related amygdala activity. This reduction was limited to subjects homozygous at two serotonin pathway-related polymorphisms, rs4570625 in the tryptophan hydroxylase-2 (TPH2) gene promoter and the long allele of the serotonin transporter-linked polymorphic region (5-HTTLPR). Although this study was limited by its small size and by not having an NCT, these findings, absent other evidence, suggest that genetic variation in serotonin pathway polymorphisms TPH2 and 5-HTTLPR might be potential biomarkers of placebo response in SAD.

Given the complex interplay of behavior, expectation, neurotransmitter signaling, disease, and the context of the medical treatment ritual, the molecular pathways and genes involved in contributing to placebo responses is unfolding as a potentially complex network.

### Placebo Associated Polymorphisms (PAPs): Main and Interaction Effects in RCT Design

It is prudent to consider issues that might arise with PAPs and the potential impact on RCT design. In general, the placebo arm is considered to be an adequate control for outcomes in the active treatment arm of RCTs. However, since placebo responses do vary by genotype, one might expect challenges with confounding, potential gene-drug-placebo effect modification and disease specific effects.

The term “efficacy” refers to the ability of a treatment to provide a clinically beneficial effect an is assessed by determining a response of a subject to the treatment. The term “drug efficacy” refers to the ability of a drug to produce a clinically beneficial effect. In a RCT, drug efficacy is determined by subtracting the primary clinical outcome in the placebo arm from outcome in the drug treatment arm.The efficacy of a drug is determined by the difference between the aggregate outcomes of individuals randomized to drug versus placebo treatment. The accuracy of the estimate of drug efficacy, especially in smaller trials, therefore depends on the randomization balancing the numbers of placebo responders by genotype across treatment arms. If by chance, in trials where the placebo response is known to be high (such as IBS), there are more genetically predisposed placebo responders in the placebo arm than in the drug arm, the estimate of drug efficacy will be confounded by genotype and the results biased towards the null. If this imbalance is not accounted for, it would be expected to be more of a problem in smaller trials than larger trials. Ideally RCTs would be designed such that the randomization balanced genetically predisposed placebo responders across all arms of a trial.

To date pharmacogenomic research has concentrated on gene-drug interactions in the context of the drug treatment. However, since many of the putative placebo genes or pathways are also drug targets, there is the possibility that these drugs could interact with the placebo response and thus compromise the assumption drug and placebo responses are additive. Furthermore, the effect of genetic variation on placebo and/or drug response, a combined gene-drug-placebo interaction, could result in differential outcomes in the placebo and drug treatment arms as a function of genotype. Although three-way interactions are considered extremely unlikely, there are several reports in the COMT literature that provide reasonable supporting evidence. For example, in a small RCT of tolcapone (a COMT inhibitor used to treat Parkinson's Disease), individuals homozygous for the low-activity COMT rs4680 met allele performed better when treated with placebo than when treated with drug. Conversely, high-activity val allele homozygotes improved with tolcapone treatment compared to placebo. These findings were interpreted as the drug “not working” for met allele homozygotes, but a gene-placebo-drug interaction hypothesis could also be applied to these differential outcomes. Although most of these studies are small and focused on mental performance outcomes, a COMT-drug-placebo effect modification was also observed in the Women's Genome Health Study, a large placebo controlled RCT of aspirin and vitamin E for the primary prevention of cardiovascular disease. In this study, not only did clinical outcomes in both the placebo and drug treatment arms vary as a function of COMT genotype, an association with baseline cardiovascular disease was also reported. Of course, without a NTC interpretation of results from the placebo arm should be approached with an abundance of caution.

The diversity of diseases associated with COMT is striking, and ranges from dopamine-associated disorders such as Parkinson's and schizophrenia, to epinephrine and norepinephrine-related disorders hypertension, pre-eclampsia, and major cardiovascular disease. COMT enzymatic activity has been shown to be inhibited by several drugs including tolcapone, quercetin and vitamin E. This potential intersection of disease, drug and placebo effects suggest that COMT is an excellent model for the sophisticated network analyses that may be necessary to fully appreciate the potential complexity of PAPs. Large-scale integration of genomic effects from proteomic, metabolomic, and small molecule-induced genome-wide transcriptional studies have greatly increased the power to identify and examine complex perturbations in these molecular networks that can compromise or enhance drug efficacy and safety. Despite the importance of placebo controls in drug development, these systems biology and pharmacology studies do not provide any data on the placebo condition. This is partly because these studies are derived in cellular model systems and partly because the concept of interaction effects between drug and placebo treatment is novel and remains to be proven. As large-scale placebo response-omics data become available, it may then be possible to identify disease and or drug specific placebo modules by mapping these molecules and their relationships to systems biology frameworks such as the interactome. An interactome often refers to the entire set of molecular interactions within a cell. The interactome therefore seeks to define the physical and biochemical influences that gene, protein, small molecule drugs, microRNA and other biomolecular networks exert on each other across normal or disease states.

The potential complexity of this network is rapidly escalated when one considers that different diseases and different placebo pathways may produce different responses in different patients. Consider, for instance, an individual who is dopaminergic-dominant and tends to be more responsive to placebo in pain studies, their placebo response in a depression trial might differ significantly depending on whether they were serotonergic-dominant or recessive. This possibility may help explain why it has been so difficult to identify consistent and reliable placebo responders. Therefore understanding the net-effect of PAPs and how this varies in the context of specific diseases and treatments may be an important consideration in personalized medicine.

While studies have not as yet been conducted to identify genes and drugs that modify placebo response, hypothetically there may even be situations in which one might opt to intentionally use a drug to modify the placebo response. For instance, purposefully using a drug to inhibit the placebo response in clinical trials could minimize the placebo response and allow for a more accurate measurement of the drug effect. In this case the placebo modifying drug would be administered to both the drug treatment and placebo arm of the trial, and any potential drug-drug or gene-drug interactions would have to be well characterized.

### Clinical Considerations

Information on whether a patient is likely to be a placebo responder or non-responder is not a disease or condition that would warrant automatic consideration in routine clinical care. PAPs seems less critical than knowing whether a singular genetic variant of a cancer will respond to particular tailored pharmaceutical interventions, yet, there may be important clinical implications in routine care. For example, compelling evidence suggests that persons homozygous for the low-activity met allele at COMT rs4680 (met/met) are more likely to respond to morphine than those homozygous for the val allele (val/val). An individual difference in morphine metabolism is the usual interpretation; however, this research is based on cohort studies of patients without placebo controls. If replication of these studies with proper placebo controls demonstrate that, in fact, this difference is due to differential placebo responses or even placebo-drug interactions, a COMT profile could be helpful in determining an initial dose for morphine treatment (and possibly other pain medications). This question of personalizing drug doses based on genetic placebo profiles is likely to be significant in conditions other than pain that are known to have high variability in both drug and placebo responses, such as functional urinary and bowel conditions and symptoms of fatigue, nausea, hot flashes, depression, and anxiety. Furthermore, the usefulness of a recently proposed strategy of open-label honest placebo treatments in such conditions as irritable bowel syndrome, acute episodic migraine attack, and depression could prove more feasible with knowledge of a patients' PAPs.

### Limitations

The ability to predict the placebo response assumes that it is a stable trait that is not influenced by the many individual states, for example personal and cultural beliefs, conscious and non-conscious expectations, previous experiences with health care, severity of illness, history of illness, and research design factors such as treatment duration, number of active arms in the trial, practitioner characteristics variables, and their interaction factors such as the quality of the entire therapeutic encounter. Therefore, these individual, contextual or situational variables present an important limitation on any simplistic or reductionist genetic model developed to predict placebo response. Although it seems plausible that genetic factors are predictive of a relative disposition to interact with such state and environmental influences, there may be epigenetic effects that are also critical to placebo responses. Furthermore, given the potential for different placebo pathways, in different classes of diseases and disorders, consideration needs to be given to developing disease or treatment specific placebo panels for specific PAP genotypes. The number of genes required to build an effective placebo response screening panel remains to be determined. With small candidate genes studies lacking NTCs, there are significant limitations to available data on PAPs. Future studies will have to be large to account for the many environmental, genetic, and drug interactions. Since in the absence of definitive studies the potential of drug treatments to interact with placebo response genes remains hypothetical, the size of these interaction effects relative to placebo effects is not known, and it remains to be seen how large a trial would have to be to measure this effect modification. In the case where interactions are significant, refinement of RCT design might be a real possibility.

### Concluding Remarks

The placebo response is a complex phenotype with an unfolding physiology. Based on the evidence summarized herein, PAPs consists of multiple intersecting pathways that have upstream or downstream effects on dopamine and opioid function, depending on the disease or disorder being treated. The endocannabinoid and serotonin pathways may also be involved, but the evidence is more limited. The potential overlap between placebo, drug treatment and disease add to the complexity of PAPs and underscore the importance of understanding how it fits into larger more complex biological networks. An important next step in describing PAPs would be to include a NTC in placebo-controlled RCTs that plan to capture-omics data. This approach might be cost-effective and allow for a broad view of placebo response genes and other molecules across varying conditions and treatments. Knowledge of PAPs has the potential to guide development of novel strategies for identifying placebo responders and clinical trial design. However numerous attendant regulatory, ethical and clinical questions would need to be addressed before such innovations could be integrated into drug development and clinical care (Box 1). Given the potential benefits in terms of research design, reduction in the cost of clinical trials, and safer more effective personalized medicines, continued PAP research is justified.

TABLE 4
Polymorphisms in candidate genes of PAPs.

| | | | Chromosomal | |
|---|---|---|---|---|
| Placebo pathway | Gene name | Symbol | location | Placebo SNPs |
| Dopamine | catechol-O-methyltransferase | COMT | 22q11.2 | rs4680 |
| | monoamine oxidase | MAO-A | Xp11.3 | rs6323, rs6609257 |
| | dopamine B hydroxylase | DBH | 9q34 | rs2873804 |
| | dopamine receptor 3 | DRD3 | 3q13.31 | rs6280 |
| | brain derived neurotropic factor | BDNF | 11p14.1 | rs6265 |
| Serotonin | tryptophan hydroxylase-2 | TPH2 | 12q21.1 | rs4570625 |
| | 5-hydroxytryptamine transporter | SLC6A4 | 17q11.2 | rs4251417 |
| | 5-hydroxytryptamine receptor 2A | HTR2A | 13q14.2 | rs2296972, rs622337 |
| | serotonin transporter gene-linked | 5-HTTLPR | 17q11.2 | Variable tandem |
| | polymorphic region | | | nucleotide repeat |
| Opioid | opioid receptor | OPRM1 | 6q25.2 | rs510769 |
| Endocannabinoid | fatty acid amide hydrolase | FAAH | 1p33 | rs324420 |

TABLE 5
PAPs

| PAP | Variants | Gene Associated With |
|---|---|---|
| rs4680 | G/A | COMT |
| rs4818 | G/C | COMT |
| rs6269 | G/A | COMT |
| rs4633 | C/T | COMT |
| rs1055503 | A/G | COMT |
| rs1109436 | C/T | COMT |
| rs11544670 | G/T | COMT |
| rs11569716 | C/T | COMT |
| rs12814 | A/T | COMT |
| rs13055002 | C/T | COMT |
| rs13306281 | A/G | COMT |
| rs1544325 | A/G | COMT |
| rs165599 | A/G | COMT |
| rs165631 | C/T | COMT |
| rs165722 | T/C | COMT |
| rs165728 | T/C | COMT |
| rs165737 | C/T | COMT |
| rs165774 | A/G | COMT |
| rs174675 | C/T | COMT |
| rs174695 | C/T | COMT |
| rs174696 | C/T | COMT |
| rs174697 | A/G | COMT |
| rs174699 | T/C | COMT |
| rs2239393 | A/G | COMT |
| rs28562050 | A/C | COMT |
| rs28565095 | A/G | COMT |
| rs28621910 | C/G | COMT |
| rs2904564 | C/G | COMT |
| rs3218737 | C/T | COMT |
| rs34068292 | C/T | COMT |
| rs34234839 | G/T | COMT |
| rs34300220 | C/T | COMT |
| rs34446512 | C/G | COMT |
| rs34450201 | A/G | COMT |
| rs34691071 | C/T | COMT |
| rs34819182 | G/T | COMT |
| rs34966851 | A/G | COMT |
| rs35478083 | C/T | COMT |
| rs35481270 | C/T | COMT |
| rs35593000 | A/C | COMT |
| rs35617967 | C/G | COMT |
| rs35777291 | A/T | COMT |
| rs35919169 | A/G | COMT |
| rs36082074 | A/G | COMT |
| rs3788319 | A/G | COMT |
| rs3788320 | A/G | COMT |
| rs3810595 | C/G | COMT |
| rs45454096 | A/G | COMT |
| rs4646312 | T/C | COMT |
| rs4646315 | C/G | COMT |
| rs4646316 | C/T | COMT |
| rs4986871 | C/T | COMT |
| rs5992500 | C/T | COMT |
| rs5993882 | T/G | COMT |
| rs5993883 | G/T | COMT |
| rs5993889 | G/T | COMT |
| rs61910731 | G/T | COMT |
| rs6267 | G/T | COMT |
| rs6268 | C/T | COMT |
| rs6270 | G/C | COMT |
| rs72549343 | —/A | COMT |
| rs737864 | C/T | COMT |
| rs737865 | A/G | COMT |
| rs737866 | C/T | COMT |
| rs739368 | A/G | COMT |
| rs740601 | G/T | COMT |
| rs740602 | A/G | COMT |
| rs740603 | G/A | COMT |
| rs769224 | A/G | COMT |
| rs9306235 | A/G | COMT |
| rs9332360 | C/T | COMT |
| rs9332367 | A/G | COMT |
| rs9332377 | C/T | COMT |
| rs9332380 | A/G | COMT |
| rs933271 | T/C | COMT |
| rs6323 | G/T | monoamine oxidase |
| rs6609257 | A/G | monoamine oxidase |
| rs2873804 | T/C | dopamine B |
| | | hydroxylase |
| rs6280 | C/T | dopamine receptor 3 |
| rs6265 | A/G | brain derived |
| | | neurotropic factor |
| rs4570625 | G/T | tryptophan |
| | | hydroxylase-2 |
| rs4251417 | A/G | 5- |
| | | hydroxytryptamine |
| | | transporter |
| rs2296972 | G/T | 5- |
| | | hydroxytryptamine |
| | | receptor 2A |
| rs622337 | A/G | 5- |
| | | hydroxytryptamine |
| | | receptor 2A |
| rs510769 | A/G | opioid receptor |
| rs324420 | A/C | fatty acid amide |
| | | hydrolase |
| rs1611115 | C/T | dopamine beta- |
| | | hydroxylase |
| rs1799971 | A/G | OPRM1 |
| rs4485648 | C/T | COMT |
| rs740601 | A/C | COMT |

TABLE 6

| | | Placebo | |
|---|---|---|---|
| Indication | COMT PAP | Allele | Other allele |
| Pain related disorders: Irritable | rs4680 | A | G |
| Bowel Syndrome, Pain, Arthritis, | rs4633 | T | C |
| Functional Disorders | rs4818 | G | C |
| Affective Disorders: Depression, | rs4680 | A | G |
| Anxiety, | rs4633 | T | C |
| | rs4818 | G | C |
| | rs740601 | G | C |
| | rs4485648 | G | T |
| Cardiometabolic diseases: | rs4680 | G | A |
| Hypertension, Diabetes, Coronary | rs4633 | C | T |
| Artery Disease, Myocardial | rs4818 | C | G |
| Infarction, Metabolic Syndrome | | | |

### Example 6

### Prophetic

A patient suffers from one of inflammatory bowel disease (IBS), depression or pain. A physician treats the patient with standard medicines/therapies. The patient has some efficacy response and some side effects. It would be beneficial if efficacy could be increased and/or side effects could be reduced. The physician tests the patient for a COMT PAP (or other PAP) and the patient tests positive for being a predicted high-placebo responder. The physician administers a placebo treatment to the patient in addition to standard therapy. The patient's efficacy response increases. The physician can now decide whether to keep the patient on standard therapy plus placebo in order to maximize treatment effect, or the physician can lower the dosage of the standard therapy in the presence of the placebo treatment to achieve adequate treatment effect and lower the severity of side effects, which is associated with the standard therapy.

Example 7

### Embodiments

A1. A method of selecting subjects to participate in a clinical trial comprising:
   identifying subjects with a catechol-O-methyltransferase (COMT) polymorphism, wherein the COMT polymorphism modulates a placebo effect in the subjects; and
   selecting subjects to participate in the clinical trial based on their COMT haplotype.
A2. The method of identifying subjects of embodiment 1, wherein the COMT polymorphism is at least one of rs4680, rs4818, rs6269, and rs4633.
A3. The method of identifying subjects of embodiment A1, wherein the COMT polymorphism encodes a valine/methionine haplotype.
A4. The method of identifying subjects of embodiment A1, wherein the COMT polymorphism encodes a methionine/methionine haplotype.
A5. The method of identifying subjects of embodiment A1, wherein the COMT polymorphism encodes a valine/valine haplotype.
A6. The method of identifying subjects of embodiment A1, wherein the COMT polymorphism indicates an increased likelihood the subject would exhibit a placebo effect.
A7. The method of identifying subjects of embodiment A1, wherein the subjects with the COMT polymorphism are excluded from the clinical trial.
A8. The method of identifying subjects of embodiment A1, wherein the subjects with the COMT polymorphism are included in the clinical trial.
A9. The method of identifying subjects of embodiment A1 further comprises modifying a treatment of the subjects with the COMT polymorphism are administered and administering the treatment.
A10. The method of identifying subjects of embodiment A1 further comprises increasing a number of subjects with the COMT polymorphism participating in the clinical trial, wherein the increase of subjects with the COMT polymorphism decreases a number of subjects needed for statistical significance of active treatment efficacy or intervention efficacy over placebo response.
A11. The method of identifying subjects of embodiment A1 further comprises a method of enriching high placebo responders in clinical trials for the purpose of achieving or demonstrating a synergistic treatment effect of active treatment efficacy or intervention efficacy and placebo response.
A12. A method for treating a subject in a clinical trial comprising:
   determining a catechol-O-methyltransferase (COMT) polymorphism in the subject;
   identifying a treatment for the subject based on the COMT polymorphism; and
   administering the treatment to the subject.
A13. The method of treating the subject of embodiment A12, wherein the step of determining the COMT polymorphism comprises:
   obtaining a sample from the subject and
   analyzing the sample for the COMT polymorphism.
A14. The method of treating the subject of embodiment A12, wherein the COMT polymorphism comprises at least one of rs4680, rs4818, rs6269, and rs4633.
A15. The method of treating the subject of embodiment A12, wherein the subject has an increased or decreased likelihood of exhibiting a placebo response.
A16. The method of treating the subject of embodiment A12, wherein the step of identifying the treatment comprises modulating the treatment for the subject with the COMT polymorphism and administering the modulated treatment.
A17. The method of treating the subject of embodiment A12, wherein the step of identifying the treatment comprises identifying an alternative treatment for the subject with the COMT polymorphism, and administering the treatment to the subject.
A18. The method of treating the subject of embodiment A12, wherein the step of administering the treatment comprises adjusting a dosage of the treatment administered to the subject depending on the COMT polymorphism.
A19. The method of treating the subject of embodiment A18, wherein the step of adjusting the treatment dosage comprises modulating the dosage to the subject with the COMT polymorphism.
A20. The method of treating the subject of embodiment A12, wherein the COMT polymorphism encodes a valine/methionine haplotype.
A21. The method of treating the subject of embodiment A12, wherein the COMT polymorphism encodes a methionine/methionine haplotype.
A22. The method of treating the subject of embodiment A12, wherein the COMT polymorphism encodes a valine/valine haplotype.
A23. A method of determining a treatment dosage for a subject in a clinical trial comprising:
   determining a catechol-O-methyltransferase (COMT) polymorphism in the subject;p and
   modulating the treatment dosage administered to the subject based on the COMT polymorphism.
A24. The method of determining the treatment dosage of embodiment A23, wherein the step of determining the COMT polymorphism comprises:
   obtaining a sample from the subject and
   analyzing the sample for the COMT polymorphism.
A25. The method of determining the treatment dosage of embodiment A23, wherein the COMT polymorphism is at least one of rs4680, rs4818, rs6269, and rs4633.
A26. The method of determining the treatment dosage of embodiment A23, wherein the step of modulating the treatment dosage administered comprises identifying an alternative treatment for the subject with the COMT polymorphism.
A27. The method of determining the treatment dosage of embodiment A23, wherein the subject with the COMT polymorphism has an increased likelihood of exhibiting a placebo response.
A28. The method of determining the treatment dosage of embodiment A27, wherein the step of modulating the treatment dosage comprises increasing the dosage for the subject with the COMT polymorphism.
A29. The method of determining the treatment dosage of embodiment A27, wherein the step of modulating the treatment dosage comprises decreasing the dosage for the subject with the COMT polymorphism.
A30. The method of determining the treatment dosage of embodiment A27, wherein the step of modulating the treatment dosage comprises identifying an alternative treatment for the subject with the COMT polymorphism.
A31. A protein or peptide assay for determining a catechol-O-methyltransferase (COMT) polymorphism in a subject.
A32. The assay of embodiment A31 comprising at least one antibody specific for the COMT polymorphism.
A33. The assay of embodiment A31 comprising reagents for measuring catechol-O-methyltransferase enzymatic activity.
A34. The assay of embodiment A31 comprising reagents for analyzing catechol-O-methyltransferase protein.
B1. A method of initiating and/or conducting a clinical trial comprising:
   a) obtaining one or more samples from each of a plurality of human subjects considered as participants for the clinical trial;
   b) analyzing the one or more samples obtained, wherein the analyzing comprises determining the presence or absence of a catechol-O-methyltransferase polymorphism for each of the plurality of human subjects;
   c) segregating the plurality of human subjects into two or more subgroups according to the analysis in b), wherein at least one of the subgroups is designated to receive an experimental treatment.
C1. A method of selecting a sub-group of human subjects likely to have a placebo response comprising:
   a) detecting the presence or absence of at least one catechol-O-methyltransferase polymorphism in a plurality of human subjects, wherein the presence of the at least one catechol-O-methyltransferase polymorphism in a subject indicates the subject is likely to have a placebo response, and
   c) selecting a sub-group of human subjects from the plurality of human subjects wherein the sub-group of human subjects comprises the presence of the catechol-O-methyltransferase polymorphism.
C2. The method of embodiment C1, further comprising evaluating the efficacy of an experimental treatment to reduce or alleviate at least one subjective symptom for each of the subgroup of human subjects selected.
C3. The method of embodiment C1 or C2, wherein the plurality of human subjects consists of candidates for a placebo-controlled clinical trial.
C4. The method of any one of embodiments C1 to C3, wherein the presence of the at least one catechol-O-methyltransferase polymorphism is associated with a placebo effect.
C5. The method of any one of embodiments C1 to C4, wherein the catechol-O-methyltransferase polymorphism comprises a single nucleotide polymorphism in a catechol-O-methyltransferase gene.
C6. The method of any one of embodiments C1 to C5, wherein the catechol-O-methyltransferase polymorphism comprises at least one of an rs4680, rs4818, rs6269 and rs4633 polymorphism.
C7. The method of any one of embodiments C1 to C6, wherein detecting the presence or absence of a catechol-O-methyltransferase polymorphism comprises determining the presence or absence of the polymorphism in one or more alleles of a catechol-O-methyltransferase gene.
C8. The method of any one of embodiments C1 to C7, wherein detecting the presence or absence of a catechol-O-methyltransferase polymorphism comprises determining an amino acid substitution in a catechol-O-methyltransferase protein.
C9. The method of embodiment C8, wherein the amino acid substitution is a valine 158 to methionine substitution.
C10. The method of any one of embodiments C1 to C7, wherein the catechol-O-methyltransferase polymorphism comprises an rs4680 polymorphism.
D1. A method of determining a treatment dosage comprising:
   determining a catechol-O-methyltransferase (COMT) polymorphism in a subject; and
   modulating the treatment dosage administered to the subject according to the COMT polymorphism.
D2. The method of determining the treatment dosage of embodiment D1, wherein the step of determining the COMT polymorphism comprises:
   obtaining a sample from the subject and
   analyzing the sample for the COMT polymorphism.
D3. The method of determining the treatment dosage of embodiment D1, wherein the step of modulating the treatment dosage administered comprises identifying an alternative treatment for the subject with the COMT polymorphism.
D4. The method of determining the treatment dosage of embodiment D1, wherein the subject with the COMT polymorphism has an increased or decreased likelihood of exhibiting a placebo response.
D5. The method of determining the treatment dosage of embodiment D4, wherein the step of modulating the treatment dosage comprises increasing the dosage for the subject with the COMT polymorphism.
D6. The method of determining the treatment dosage of embodiment D4, wherein the step of modulating the treatment dosage comprises decreasing the dosage for the subject with the COMT polymorphism.
D7. The method of determining the treatment dosage of embodiment D1, wherein the subject is suspected of having, or is at risk for developing at least one of irritable bowel syndrome, diabetes, and cardiovascular disease.
D8. The method of determining the treatment dosage of embodiment D1, wherein the COMT polymorphism comprises at least one of rs4680, rs4818, rs6269, and rs4633.
D9. A method for treating a subject comprising:
   determining a catechol-O-methyltransferase (COMT) polymorphism in the subject;
   identifying a treatment for the subject based on the COMT polymorphism; and
   administering the treatment to the subject.
D10. The method of treating the subject of embodiment D9, wherein the subject has, is suspected of having, or is at risk for developing a disorder of at least one of irritable bowel syndrome, diabetes, and a cardiovascular disease.
D11. The method of treating the subject of embodiment D9, wherein the step of determining the COMT polymorphism comprises:
   obtaining a sample from the subject and
   analyzing the sample for the COMT polymorphism.
D12. The method of treating the subject of embodiment D9, wherein the COMT polymorphism comprises at least one of rs4680, rs4818, rs6269, and rs4633.
D13. The method of treating the subject of embodiment D9, wherein the subject has an increased or decreased likelihood of exhibiting a placebo effect.
D14. The method of treating the subject of embodiment D9, wherein the step of identifying the treatment comprises modulating the treatment for the subject with the COMT polymorphism and administering the modulated treatment.
D15. The method of treating the subject of embodiment D9, wherein the step of identifying the treatment comprises identifying an alternative treatment for the subject with the COMT polymorphism, and administering the treatment to the subject.
D16. The method of treating the subject of embodiment D9, wherein the step of administering the treatment comprises adjusting a dosage of the treatment administered to the subject depending on the COMT polymorphism.
D17. The method of treating the subject of embodiment D16, wherein the step of adjusting the dosage treatment comprises increasing or decreasing the dosage to the subject with the COMT polymorphism.
D18. The method of treating the subject of embodiment D9, wherein the COMT polymorphism encodes a valine/methionine haplotype.
D19. The method of treating the subject of embodiment D9, wherein the COMT polymorphism encodes a methionine/methionine haplotype.
D20. The method of treating the subject of embodiment D9, wherein the COMT polymorphism encodes a valine/valine haplotype.
D21. A protein or peptide assay for determining a catechol-O-methyltransferase (COMT) polymorphism in a subject.
D22. The assay of embodiment D21 comprising at least one antibody specific for the COMT polymorphism.
D23. The assay of embodiment D21 comprising reagents for measuring catechol-O-methyltransferase enzymatic activity.
D24. The assay of embodiment D21 comprising reagents for analyzing catechol-O-methyltransferase protein.
D25. The method of any one of embodiments D1 to D20, wherein the treatment comprises administering a drug that interacts with an opioid pathway involved with a placebo response.
D26. The method of D25, wherein the drug is naloxone, a pain medications, ketorolac, an opiate, or buprenorphine.
D27. The method of any one of embodiments D1 to D20, wherein the treatment comprises administering a drug that interact in a serotonin pathway.
D28. The method of D25, wherein the drug is selective serotonin reuptake inhibitor or a tricyclic.
E1. A method of conducting a randomized clinical trial comprising:
   a) detecting the presence or absence of a placebo-associated polymorphism in a sub-population of human subjects, wherein the human subjects are candidates for a clinical trial, thereby providing a first sub-group of subjects comprising the absence of a placebo-associated polymorphism and a second sub-group of subjects comprising the presence of placebo-associated polymorphism;
   b) distributing the first sub-group into at least a first study group and a second study group, wherein at least the first study group is administered a first treatment;
   c) measuring a response in the subjects of the first and second study groups;
   d) evaluating the efficacy of the first treatment by comparing the measured response of one or more subjects of the first and second study groups.
E2. The method of embodiment E1, wherein the second study group is administered a placebo treatment.
E3. The method of embodiment E1 or E2, wherein the first and second study groups are administered a second treatment.
E4. The method of any one of embodiments E1 to E3, wherein a subject comprising the presence of the placebo-associated polymorphism is homozygous for the presence of the placebo-associated polymorphism.
E5. The method of any one of embodiments E1 to E3, wherein a subject comprising the presence of the placebo-associated polymorphism is heterozygous for the presence of the placebo-associated polymorphism.
E6. The method of any one of embodiments E1 to E5, wherein the second subgroup is distributed evenly and/or randomly among at least the first and second study groups.
E7. The method of any one of embodiments E1 to E6, wherein the second subgroup is tracked within the first and second study groups.
E8. The method of any one of embodiments E1 to E5, wherein the second subgroup is removed from the clinical trial.
E9. The method of any one of embodiments E1 to E8, wherein the placebo-associated polymorphism comprises a COMT polymorphism.
E10. The method of embodiment E9, wherein the COMT polymorphism is selected from the group consisting of rs4680, rs4818, rs6269 and rs4633.
E11. The method of any one of embodiments E1 to E8, wherein the placebo-associated polymorphism is selected from the group consisting of rs6323, rs6609257, rs2873804, rs6280, rs6265, rs4570625, rs4251417, rs2296972, rs622337, rs510769, rs324420, rs1611115, and rs1799971.
E12. The method of any one of embodiments E1 to E11, wherein the first experimental treatment comprises administering a pharmaceutical composition for the treatment of irritable bowel syndrome, diabetes, an autoimmune disorder, inflammation, a neurological disorder, chronic and acute pain, cancer, allergies, depression, migraines, addiction, obesity or cardiovascular disease.
E13. The method of any one of embodiments E1 to E12, wherein the subpopulation of human subjects has a disorder selected from the group of irritable bowel syndrome, diabetes, an autoimmune disorder, inflammation, a neurological disorder, chronic and acute pain, cancer, allergies, depression, migraines, addiction, obesity, and cardiovascular disease.
E14. The method of any one of embodiments E1 to E13, wherein the first experimental treatment comprises administering vitamin E and/or aspirin and the measured response is a clinical characteristic of cardiovascular disease.
E15. The method of embodiment E14, wherein the clinical characteristic of cardiovascular disease is selected from a frequency of myocardial infarction and stroke.
E16. The method of embodiment E14, wherein the clinical characteristic of cardiovascular disease is selected from one or more of a systolic blood pressure, diastolic blood pressure, serum triglycerides, serum apolipoprotein B and serum soluble intracellular adhesion molecule I.
E17. The method of any one of embodiments E1 to E13, wherein the first experimental treatment comprises administering an analgesic and the measured response is a sensation of pain.
E18. The method of any one of embodiments E1 to E13, wherein the first experimental treatment comprises administering a pharmaceutical composition for the treatment of irritable bowel syndrome and the measured response is a clinical characteristic of irritable bowel syndrome.
E19. The method of embodiment E18, wherein the clinical characteristic of irritable bowel syndrome is selected from the group consisting of abdominal pain severity, abdominal pain frequency, abdominal distention severity, dissatisfaction with bowel habits, and disruption in quality of life.
E20. The method of any one of embodiments E1 to E19, wherein the first treatment comprises administering a drug that interacts with an opioid pathway involved with a placebo response.
E21. The method of embodiment E20, wherein the drug is naloxone, a pain medication, ketorolac, an opiate, or buprenorphine.
E22. The method of any one of embodiments E1 to E20, wherein the first treatment comprises administering a drug that interacts with a serotonin pathway.
E23. The method of embodiment E22, wherein the drug is a selective serotonin reuptake inhibitor or a tricyclic.
E24. The method of any one of embodiments E1 to E23, wherein the measured response comprises a reduction in a symptom selected from pain, depression, and psychosis.
E25. The method of any one of embodiments E1 to E23, wherein the measured response comprises a reduction in blood pressure.
E26. The method of any one of embodiments E1 to E20, wherein the first treatment comprises administering a drug that binds directly to a COMT protein.
E27. The method of any one of embodiments E1 to E20, wherein the first treatment comprises administering a drug that inhibits an activity or function of a COMT protein.
E28. The method of embodiment E26 or E27, wherein the drug is selected from quercetin, S-adenosylmethionine, tolcapone, entacapone, a beta-blocker, an alpha adrenergic receptor blocker, a beta-adrenergic receptor blocker, a agonist of alpha adrenergic receptor blocker, a agonist of beta-adrenergic receptor blocker, vitamin A, vitamin C, vitamin D, vitamin E and levodopa.
E30. The method of embodiment E1, wherein the placebo-associated polymorphism is selected from the placebo-associated polymorphisms in Table 5.
F1. A method of treating a subject having or suspected of having a disorder comprising:
   a) determining a presence or absence of a placebo-associated polymorphism in the subject, wherein the presence of the polymorphism is associated with an increased likelihood of the subject having a placebo response;
   b) administering an amount of a treatment to the subject wherein said treatment is indicated for the disorder, and
   c) administering a placebo treatment to the subject.
F2. The method of embodiment F1, wherein the subject is suspected of having, or is at risk of having at least one of irritable bowel syndrome, diabetes, pain or cardiovascular disease.
F3. The method of embodiment F1 or F2, wherein the treatment comprises administering a drug that interacts with an opioid pathway involved with a placebo response.
F4. The method of embodiment F3, wherein the drug is naloxone, a pain medications, ketorolac, an opiate, or buprenorphine.
F5. The method of embodiment F1 or F2, wherein the treatment comprises administering a drug that interacts with a serotonin pathway.
F6. The method of embodiment F5, wherein the drug is a selective serotonin reuptake inhibitor or a tricyclic.
F7. The method of embodiment F1 or F2, wherein the treatment comprises administering a drug that binds directly to a COMT protein.
F8. The method of embodiment F1 or F2, wherein the treatment comprises administering a drug that inhibits an activity or function of a COMT protein.
F9. The method of embodiment F7 or F8, wherein the drug is selected from quercetin, S-adenosylmethionine, tolcapone, entacapone, a beta-blocker, an alpha adrenergic receptor blocker, a beta-adrenergic receptor blocker, a agonist of alpha adrenergic receptor blocker, a agonist of beta-adrenergic receptor blocker, vitamin A, vitamin C, vitamin D, vitamin E and levodopa.
F10. The method of embodiment F1, wherein the placebo-associated polymorphism is selected from the placebo-associated polymorphisms listed in Table 5.
G1. A method of identifying a placebo-associated polymorphism associated with a differential response to a treatment comprising:
   a) detecting the presence or absence of a placebo-associated polymorphism in a sub-population of human subjects, wherein the human subjects have, or are at risk of having a disorder, thereby providing a first sub-group of subjects comprising the presence of the placebo-associated polymorphism and a second sub-group of subjects comprising the absence of the placebo-associated polymorphism;
   b) distributing the first sub-group evenly among at least a first study group and a second study group;
   c) distributing the second sub-group evenly among at least the first study group and the second study group;
   d) administering the treatment to the first study group and a control treatment to the second study group;
   e) determining a response to the treatment, wherein the determining comprises comparing the response of one or more subjects of the first study group with the response of one or more subjects of the second study groups; and
   f) comparing the response determined in (e) between i) one or more subjects of the first subgroup in the first study group with ii) or more subjects of the second subgroup in the first study group, wherein a statistically significant difference in the response between i) and ii) indicates the polymorphism is associated with a differential response to the treatment.
G2. A method of identifying a polymorphism associated with a differential response to a treatment comprising:
   a) identifying a first sub-group of human subjects comprising the presence of a placebo-associated polymorphism and a second sub-group of human subjects comprising the absence of a placebo-associated polymorphism, wherein the human subjects have, or are at risk of having a disorder;
   b) administering a treatment to the first sub-group and the second sub-group;
   c) determining a response to the treatment for i) one or more subjects of the first sub-group and for ii) one or more subjects of the second sub-group; and
   f) comparing the response between i) and ii), wherein a statistically significant difference in the response between i) and ii) indicates the polymorphism is associated with a differential response to the treatment.
G3. The method of any one of embodiments G1 to G2, wherein a subject comprising the presence of the placebo-associated polymorphism is homozygous for the presence of the placebo-associated polymorphism.
G4. The method of any one of embodiments G1 to G2, wherein a subject comprising the presence of the placebo-associated polymorphism is heterozygous for the presence of the placebo-associated polymorphism.
G5. The method of embodiment G1, wherein the distributing in b) is random.
G6. The method of embodiment G1, wherein the distributing in c) is random.
G7. The method of any one of embodiments G1 to G6, wherein the method comprises blocked randomization.
G8. The method of any one of embodiments G1 to G7, wherein the placebo-associated polymorphism comprises a COMT polymorphism.
G9. The method of embodiment G8, wherein the COMT polymorphism is selected from the group consisting of rs4680, rs4818, rs6269 and rs4633.
G10. The method of any one of embodiments G1 to G7, wherein the placebo-associated polymorphism is selected from the group consisting of rs6323, rs6609257, rs2873804, rs6280, rs6265, rs4570625, rs4251417, rs2296972, rs622337, rs510769, rs324420, rs1611115, and rs1799971.
G11. The method of any one of embodiments G1 to G10, wherein the treatment is an experimental treatment.
G12. The method of any one of embodiments G1 to G11, wherein the treatment comprises administering a pharmaceutical composition for the treatment of irritable bowel syndrome, diabetes, an autoimmune disorder, inflammation, a neurological disorder, chronic and acute pain, cancer, allergies, depression, migraines, addiction, obesity or cardiovascular disease.
G13. The method of any one of embodiments G1 to G12, wherein the human subjects in the first subgroup and second subgroup have a disorder selected from the group of irritable bowel syndrome, diabetes, an autoimmune disorder, inflammation, a neurological disorder, chronic and acute pain, cancer, allergies, depression, migraines, addiction, obesity, and cardiovascular disease.
G14. The method of any one of embodiments G1 to G13, wherein the treatment comprises administering vitamin E and/or aspirin and the response is a clinical characteristic of cardiovascular disease.
G15. The method of embodiment G14, wherein the clinical characteristic of cardiovascular disease is selected from a frequency of myocardial infarction and stroke.
G16. The method of embodiment G14, wherein the clinical characteristic of cardiovascular disease is selected from one or more of a systolic blood pressure, diastolic blood pressure, serum triglycerides, serum apolipoprotein B and serum soluble intracellular adhesion molecule I.
G17. The method of any one of embodiments G1 to G13, wherein the treatment comprises administering an analgesic and the measured response is a sensation of pain.
G18. The method of any one of embodiments G1 to G13, wherein the treatment comprises administering a pharmaceutical composition for the treatment of irritable bowel syndrome and the measured response is a clinical characteristic of irritable bowel syndrome.
G19. The method of embodiment G18, wherein the clinical characteristic of irritable bowel syndrome is selected from the group consisting of abdominal pain severity, abdominal pain frequency, abdominal distention severity, dissatisfaction with bowel habits, and disruption in quality of life.
G20. The method of any one of embodiments G1 to G19, wherein the treatment comprises administering a drug that interacts with an opioid pathway involved with a placebo response.
G21. The method of embodiment G20, wherein the drug is naloxone, a pain medication, ketorolac, an opiate, or buprenorphine.
G22. The method of any one of embodiments G1 to G19, wherein the first treatment comprises administering a drug that interacts with a serotonin pathway.
G23. The method of embodiment G22, wherein the drug is a selective serotonin reuptake inhibitor or a tricyclic.
G24. The method of any one of embodiments G1 to G23, wherein the measured response comprises a reduction in a symptom selected from pain, depression, and psychosis.
G25. The method of any one of embodiments G1 to G23, wherein the measured response comprises a reduction in blood pressure.
G26. The method of any one of embodiments G1 to G19, wherein the treatment comprises administering a drug that binds directly to a COMT protein.
G27. The method of any one of embodiments G1 to G26, wherein the treatment comprises administering a drug that inhibits an activity or function of a COMT protein.
G28. The method of embodiment G26 or G27, wherein the drug is selected from quercetin, S-adenosylmethionine, tolcapone, entacapone, a beta-blocker, an alpha adrenergic receptor blocker, a beta-adrenergic receptor blocker, a agonist of alpha adrenergic receptor blocker, a agonist of beta-adrenergic receptor blocker, vitamin A, vitamin C, vitamin D, vitamin E and levodopa.
G29. The method of embodiment G1, wherein the placebo-associated polymorphism is selected from the placebo-associated polymorphisms listed in Table 5.
H1. A method of identifying a placebo-associated polymorphism associated with a differential response to a placebo treatment comprising:
   a) detecting the presence or absence of a placebo-associated polymorphism in a sub-population of human subjects, wherein the human subjects have, or are at risk of having a disorder or condition, thereby providing a first sub-group of subjects comprising the presence of the placebo-associated polymorphism and a second sub-group of subjects comprising the absence of the placebo-associated polymorphism;
   b) distributing the first sub-group evenly among at least a first study group and a second study group;
   c) administering a treatment to the first and second study groups, wherein the treatment is indicated for the disorder or condition, the treatment is administered in substantially the same amount and substantially in the same way, and wherein the efficacy of the treatment can be assessed by measuring at least one response;
   d) administering a placebo treatment to the second study group;
   e) measuring the at least one response in one or more subjects of the first and second study groups;
   f) comparing the response measured in e) between i) one or more subjects of the first the first study group and ii) one or more subjects of the second subgroup, wherein a statistically significant difference in the response between i) and ii) indicates the placebo-associated polymorphism is associated with a differential response to the placebo treatment.

What is claimed is:

1. A method of conducting a randomized clinical trial comprising:
a) detecting a genotype of a placebo-associated polymorphism in a sub-population of human subjects, wherein the human subjects are candidates for a clinical trial, thereby providing a first sub-group of subjects comprising a first genotype of the polymorphism and a second sub-group of subjects comprising a second genotype of the polymorphism, wherein the first and second genotypes are not the same;
b) distributing the first sub-group evenly, unevenly and/or randomly into at least a first study group and a second study group, wherein at least the first study group is administered a first treatment and the second study group is administered a placebo treatment.

2. The method of claim 1, wherein after administering the first treatment and the placebo treatment, a response is measured from the subjects of the first and second study groups, and wherein the safety and/or efficacy of the first treatment is evaluated by comparing the measured response between one or more subjects of the first and second study groups.

3. The method of claim 1, wherein the second subgroup is excluded from at least the first and second study groups.

4. The method of claim 1, wherein the second subgroup is excluded from participating in the randomized clinical trial.

5. The method of claim 1, wherein the second genotype is associated with an enhanced placebo response.

6. The method of claim 1, wherein the second subgroup is distributed evenly, unevenly and/or randomly into at least the first study group and the second study group.

7. The method of claim 1, further providing a third sub-group of subjects comprising a third genotype of the polymorphism, wherein the third genotype is different than the first and the second genotype.

8. The method of claim 7, wherein the third sub-group is associated with an enhanced placebo response and wherein the third sub-group is excluded from at least the first and second study groups.

9. The method of claim 7, wherein the third sub-group is distributed evenly, unevenly and/or randomly into at least the first study group and the second study group.

10. The method of claim 1, wherein the first genotype comprises a homozygous variant of the polymorphism or a heterozygous variant of the polymorphism.

11. The method of claim 1, wherein the second genotype comprises a homozygous variant of the polymorphism or a heterozygous variant of the polymorphism.

12. The method of claim 1, wherein the placebo-associated polymorphism is selected from a placebo-associated polymorphisms in Table 5.

13. The method of claim 12, wherein the placebo-associated polymorphism is a COMT polymorphism.

14. The method of claim 13, wherein the COMT polymorphism is selected from the group consisting of rs4680, rs4818, rs6269, rs4633, rs4485648 and rs740601.

15. The method of claim 12, wherein the placebo-associated polymorphism is selected from the group consisting of rs6323, rs6609257, rs2873804, rs6280, rs6265, rs4570625, rs4251417, rs2296972, rs622337, rs510769, rs324420, rs1611115, and rs1799971.

16. The method of claim 1, wherein the first experimental treatment comprises administering a pharmaceutical composition for the treatment of a disorder or a condition selected from the group consisting of irritable bowel syndrome, diabetes, an autoimmune disorder, inflammation, a neurological disorder, chronic or acute pain, cancer, allergies, depression, migraines, addiction, obesity and cardiovascular disease.

17. The method of claim 2, wherein the response is a symptom or clinical characteristic of the disorder or condition.

18. The method of claim 1, wherein the first experimental treatment comprises administering an analgesic, a drug that interacts with an opioid pathway, a drug that interacts with a serotonin pathway, a drug that binds directly to a COMT protein, a drug that inhibits an activity or function of a COMT protein, a beta-blocker, an alpha-blocker, an agonist of alpha adrenergic receptor, or a agonist of beta-adrenergic receptor blocker.

19. The method of claim 1, wherein the second genotype comprises a placebo allele listed in Table 6.

20. The method of claim 1, wherein the genotype is selected from an A/A homozygote, A/G heterozygote and G/G homozygote of an rs4680 COMT polymorphism.

21. A method of treating a subject having a placebo-associated polymorphism with a placebo comprising:
a) administering a treatment to a human subject having, or are at risk of having a disorder or condition, and wherein the treatment is indicated for the disorder or condition;
b) determining an efficacy of the treatment according to a response of the human subject to the treatment, wherein the response is a clinical characteristic or symptom of the disease or disorder;
c) determining a genotype for a placebo-associated polymorphism in the subject; and
d) administering the treatment and a placebo treatment to the subject if the genotype is associated with an enhanced placebo effect.

22. The method of claim 21, comprising after d), repeating the determining step of b), and if the efficacy determined is substantially greater than the efficacy determined in b), then either i) continue administering the treatment and placebo treatment to the subject as needed or ii) continue administering the placebo treatment and administer a lower dosage of the treatment.

23. The method of claim 21, wherein the placebo-associated polymorphism comprises a COMT polymorphism.

24. The method of claim 23, wherein the COMT polymorphism is selected from the group consisting of rs4680, rs4818, rs6269, rs4633, rs4485648 and rs740601.

25. The method of claim 21, wherein the placebo-associated polymorphism is selected from the group consisting of rs6323, rs6609257, rs2873804, rs6280, rs6265, rs4570625, rs4251417, rs2296972, rs622337, rs510769, rs324420, rs1611115, and rs1799971.

26. The method of claim 21, wherein the treatment comprises administering a pharmaceutical composition for the treatment of a disorder or a condition selected from the group consisting of irritable bowel syndrome, diabetes, an autoimmune disorder, inflammation, a neurological disorder, chronic or acute pain, cancer, allergies, depression, migraines, addiction, obesity and cardiovascular disease.

27. A method of treating a subject having a placebo-associated polymorphism with a placebo comprising:
a) administering a treatment to a human subject having, or are at risk of having a disorder or condition, and wherein the treatment is indicated for the disorder or condition;
b) determining an efficacy, and a presence or degree of an adverse effect of the treatment according to one or more responses of the human subject to the treatment;
c) determining a genotype for a placebo-associated polymorphism in the subject; and
d) if the genotype is associated with an enhanced placebo effect, administering the treatment and a placebo treatment to the subject, wherein the amount or dosage of the treatment is reduced compared to the amount administered in a).

28. The method of claim 27, comprising after d), repeating the determining step of b), and
if i) the efficacy determined is the same or greater than the efficacy determined in b), and
ii) the presence or degree of the adverse effect is eliminated or reduced compared to the presence or degree of the adverse effect determined in b), then continue administering the reduced treatment and placebo treatment to the subject as needed.

29. A method of identifying a genotype of a placebo associated polymorphism that is associated with an enhanced placebo response comprising:
a) detecting two or more genotypes of a placebo-associated polymorphism in a sub-population of human subjects, wherein the human subjects have or are suspected of having a disorder or condition, thereby providing a first sub-group and second sub-group, the first sub-group of subjects comprising a first genotype of the polymorphism and a second sub-group of subjects comprising a second genotype of the polymorphism, wherein the first and second genotypes are not the same;
b) administering a substantially same placebo treatment to one or more subjects of the first and second sub-groups;
c) measuring a response of one or more subjects in the first and second sub-groups, wherein the response is a clinical characteristic or symptom of the disorder or condition; and
d) comparing the response to the placebo treatment between one or more subjects of the first and second sub-groups, wherein an improvement in the response of one or more subjects in the first sub-group compared to the second sub-group indicates the genotype of the placebo associated polymorphism of the first sub-group is associated with an enhanced placebo response.

30. The method of claim 29, wherein an improvement in the response is a reduction or elimination of one or more adverse symptoms.

31. A method of identifying a genotype of a placebo associated polymorphism that is associated with an enhanced treatment response comprising:
a) detecting two or more genotypes of a placebo-associated polymorphism in a sub-population of human subjects, wherein the human subjects have or are suspected of having a disorder or condition that is treatable by a first treatment, thereby providing a first sub-group and second sub-group, the first sub-group of subjects comprising a first genotype of the polymorphism and a second sub-group of subjects comprising a second genotype of the polymorphism, wherein the first and second genotypes are not the same;
b) administering a substantially same treatment to one or more subjects of the first and second sub-groups;
c) measuring a response of one or more subjects in the first and second sub-groups, wherein the efficacy and/or safety of the treatment is determined by measuring the response; and
d) comparing the efficacy and/or safety of the treatment between one or more subjects of the first and second sub-groups, wherein an increase in the efficacy and/or safety of one or more subjects in the first sub-group compared to the second sub-group indicates the genotype of the placebo associated polymorphism of the first sub-group is associated with an enhanced treatment response to the first treatment.

32. The method of claim 31, wherein the subjects of the first and second sub-groups are distributed evenly, unevenly or randomly among two or more study groups.

33. The method of claim 32, wherein the response of each subject of the first and second sub-groups is individually tracked and/or monitored.


SEQUENCE LISTING (77 sequences)

SEQ ID NO: 1 - DNA, 52 bp, Homo sapiens
aagggaggggagggggccccgacggaggccacagtaaaggagtggaggggcc

SEQ ID NO: 2 - DNA, 52 bp, Homo sapiens
tgcaattcaaaatcaagggctgcttctgaggaggcctctccaccgggctgct

SEQ ID NO: 3 - DNA, 52 bp, Homo sapiens
tgccggaggccccgcctctgctgttgtgcagctgtgttgctgggcctggtgc

SEQ ID NO: 4 - DNA, 52 bp, Homo sapiens
tccgtgtctgcttctgtattttgtgctggttttagaggatccctgggctgcc

SEQ ID NO: 5 - DNA, 52 bp, Homo sapiens
cccggcccccctctcgggctctctcatcccagcctggtactgaaggtgccag

SEQ ID NO: 6 - DNA, 52 bp, Homo sapiens
tggccccaggggctaggcacaggcgctggtgccgtggcctagtgaggagcac

SEQ ID NO: 7 - DNA, 52 bp, Homo sapiens
cgagcagaaggagtgggccatgaacagtgggcgacaagaaaggtggggtccg

SEQ ID NO: 8 - DNA, 52 bp, Homo sapiens
gagacaaattagaaatgtcagtctgagagagagtggtaggtagccagatact

SEQ ID NO: 9 - DNA, 52 bp, Homo sapiens
tgttagccccatggggacgactgccaggcctgggaaacgaagaggagtcagc

SEQ ID NO: 10 - DNA, 52 bp, Homo sapiens
accggtacctgccggacacgcttctctttggaggtgagccccaaccaggatg

SEQ ID NO: 11 - DNA, 52 bp, Homo sapiens
cttccctgttctcttctgctctgtctctctggtgccctgaggctggcctcca

SEQ ID NO: 12 - DNA, 52 bp, Homo sapiens
agtgaggatgcagtgctggtttctgtcccacctacacctagagctgtcccca

SEQ ID NO: 13 - DNA, 52 bp, Homo sapiens
ttgggacacccaccctcacggcctcctccacctggtgctcgctcacctgcag

SEQ ID NO: 14 - DNA, 52 bp, Homo sapiens
aaactggacactgctgttagcagccaggactaggagcacgaggggcacagcc

SEQ ID NO: 15 - protein, 52 aa, Homo sapiens
GAAATGCTGTCAGAAGCCTACCCCACTGGTACCTGTCATGGGCCTTTTCATT

SEQ ID NO: 16 - DNA, 52 bp, Homo sapiens
gggacaggcgggcactgggtgcctcctttgcaccagccaggcccagcctgca

SEQ ID NO: 17 - DNA, 52 bp, Homo sapiens
ctgcgtccggccgtattccagctttctaaaacaacaaaaaacaacaaaaact

SEQ ID NO: 18 - DNA, 52 bp, Homo sapiens
gggttctgtgagcatcggaggcacgagggggtgaggggctcaggagcaggtt

SEQ ID NO: 19 - DNA, 52 bp, Homo sapiens
ctcctctgacactgtcgcttctccatcggcattagattttcagtcctgctca

SEQ ID NO: 20 - DNA, 52 bp, Homo sapiens
ttacaggagaagctgttatcaccccagtttccagggggctgggaaccctggg

SEQ ID NO: 21 - DNA, 52 bp, Homo sapiens
gaggtttctcccgatagtggaccccaccgggccatgccctatggctgcgtgt

SEQ ID NO: 22 - DNA, 52 bp, Homo sapiens
tggggagccagctgcctgtgcaggtagcaaaatgggtggcagaagtggggtg

SEQ ID NO: 23 - DNA, 52 bp, Homo sapiens
ccatgcaggtctggcccatggaaggcgaggggagggggccccgacggggcca

SEQ ID NO: 24 - DNA, 52 bp, Homo sapiens
cggactaggagcacgaggggcacagcgccccatgcctggctaggtagggccg

SEQ ID NO: 25 - DNA, 52 bp, Homo sapiens
cgtgggcgacaagaaaggtggggtcctgggccagcaggtgctcagctctggg

SEQ ID NO: 26 - DNA, 52 bp, Homo sapiens
agccactgctcgtgcaaagaaagcactgtgtctcctgcagatcttcctcctg

SEQ ID NO: 27 - DNA, 52 bp, Homo sapiens
tcctctggagtcccagggtgccagggttccctgatgacccctgcaggccctg

SEQ ID NO: 28 - DNA, 52 bp, Homo sapiens
ctgggcccaagcaatcctcccacttctagcctcctgagtagctgcagctata

SEQ ID NO: 29 - DNA, 52 bp, Homo sapiens
gctctgggcccagcctgctctcccccgaagcaagccactgctcgtgcaaaga

SEQ ID NO: 30 - DNA, 52 bp, Homo sapiens
gccttctgctgtctcttctgaggccaggaaaggcgaagcactgccctcgccc

SEQ ID NO: 31 - DNA, 52 bp, Homo sapiens
gagctggagggggggtcttcctgggctggcctgtgtagcagcagctccaggc

SEQ ID NO: 32 - DNA, 52 bp, Homo sapiens
ctctgggacagggacccaggaccaggtcatcaaagcccttacaggagaagct

SEQ ID NO: 33 - DNA, 52 bp, Homo sapiens
atctggatttgatggcatttttattagtggtgttgttttagagacagtgtct

SEQ ID NO: 34 - DNA, 52 bp, Homo sapiens
gcagcaccagcccctctgggccccactgtggcacagagtggaagcatctcct

SEQ ID NO: 35 - DNA, 52 bp, Homo sapiens
agaggcaatggctcagaccagctccctgcatccctgtagttgcctccctggc

SEQ ID NO: 36 - DNA, 52 bp, Homo sapiens
ttgcttacagaagaggcaatggctcacgaccagctcccgcatccctgtagtt

SEQ ID NO: 37 - DNA, 52 bp, Homo sapiens
gggctctggcctttggccttttcctcgtgagctgcatgttggccactgtgac

SEQ ID NO: 38 - DNA, 52 bp, Homo sapiens
ctgaaaacatcttgtaagcttttttatggccaatatattatcaaacgcaatt

SEQ ID NO: 39 - DNA, 52 bp, Homo sapiens
ggaccttggtcatcgccaggttaggagtttatgttgatgattatccaaaggc

SEQ ID NO: 40 - DNA, 52 bp, Homo sapiens
ctggggacaggtggaccccgcagcaagcaccagcccctctgggccccatgtg

SEQ ID NO: 41 - DNA, 52 bp, Homo sapiens
gtgaagtgatctgacgttgggtgggaggtctccggacttggggtggggaatc

SEQ ID NO: 42 - DNA, 52 bp, Homo sapiens
gagggccttggtgacttctccaacaaggctccctgttggaggccttggggta

SEQ ID NO: 43 - DNA, 52 bp, Homo sapiens
tacagcctctcctttggccacccgtcgactacccccaactccgggccatggg

SEQ ID NO: 44 - DNA, 52 bp, Homo sapiens
actgggatagggtgtggggaattcgagaccgctgtgaagtgatctgacgttg

SEQ ID NO: 45 - DNA, 52 bp, Homo sapiens
ccaaggagcagcgcatcctgaaccactgtgctgcagcatgcggagcccggga

SEQ ID NO: 46 - DNA, 52 bp, Homo sapiens
tggtttgtgtatgttcttggtaaactcagcccttggtcttacacatcatttc

SEQ ID NO: 47 - DNA, 52 bp, Homo sapiens
ttcagtcagcctcagcctctccaaacgagccaggcattccagtagagccctg

SEQ ID NO: 48 - DNA, 52 bp, Homo sapiens
cccagaccagacaccagggcagaaactggcacaggaccaaggagatggggtg

SEQ ID NO: 49 - DNA, 52 bp, Homo sapiens
ccagcggatggtggatttcgctggcagtgaaggacaaggtgtgcatgcctga

SEQ ID NO: 50 - DNA, 52 bp, Homo sapiens
gcctgctgtcaccaggggcgaggctcgatcaccatcgagatcaaccccgact

SEQ ID NO: 51 - DNA, 52 bp, Homo sapiens
accatcgagatcaaccccgactgtgctcgccatcacccagcggatggtggat

SEQ ID NO: 52 - DNA, 52 bp, Homo sapiens
tgccatggtcggatgagagcagccactggacaatgtctgtgcaattgtgcgt

SEQ ID NO: 53 - DNA, 52 bp, Homo sapiens
aaagttacgcttaataatgaatgtttgcagcactttcttctcttcaggtatt

SEQ ID NO: 54 - DNA, 52 bp, Homo sapiens
gttgaaagttactgaaaacatcttggtaagcttttttaggccaatatattat

SEQ ID NO: 55 - DNA, 52 bp, Homo sapiens
gtaagcaaacccttttctgatagcagtagataggcaagcatcctatgaggtt

SEQ ID NO: 56 - DNA, 52 bp, Homo sapiens
tccccaaccctgcacaggcaagatcgttggacgccgtgattcaggagcacca

SEQ ID NO: 57 - DNA, 52 bp, Homo sapiens
gctgcagcatgcggagcccgggaacgtcacagagcgtgctggaggccattga

SEQ ID NO: 58 - DNA, 52 bp, Homo sapiens
gcaccagagggcacgagaaggctggcttccctggcgctgacacgtcaggcaa

SEQ ID NO: 59 - DNA, 52 bp, Homo sapiens
gcatttctgaaccttgcccctctgcgaaacacaagggggcgatggtggcact

SEQ ID NO: 60 - DNA, 52 bp, Homo sapiens
ctgaggcactggggctggggcctgtgcccttatcggctggaacgagttcatc

SEQ ID NO: 61 - DNA, 51 bp, Homo sapiens
tcctgcagcccatccacaacctgctacatgggtgacaccaaggagcagcgc

SEQ ID NO: 62 - DNA, 52 bp, Homo sapiens
gttgctttttattgtttaaagtgacctctccaagcctgagggaccgtaggag

SEQ ID NO: 63 - DNA, 52 bp, Homo sapiens
gctttttggatttttccagccagggagtttttgtgtcctgttgctttttatt

SEQ ID NO: 64 - DNA, 52 bp, Homo sapiens
gctaacagacctgctttttggatttcttccagccagggatttttgtgtcctg

SEQ ID NO: 65 - DNA, 52 bp, Homo sapiens
actgtgtgctccggttgttgcatccagagggttgatcggatggtggttccca

SEQ ID NO: 66 - DNA, 52 bp, Homo sapiens
cagggccccaaggtgggcggttcgggtgattcagagagggcagctctgtgtt

SEQ ID NO: 67 - DNA, 52 bp, Homo sapiens
agcatgcggagcccgggaacgcacaagagcgtgctggaggccattgacacct

SEQ ID NO: 68 - DNA, 52 bp, Homo sapiens
tgtgaggcactgaggatgccctcacgacgtgcatctgcatgtggcgtgcatg

SEQ ID NO: 69 - DNA, 52 bp, Homo sapiens
accactggaaggaccggtacctgccaggacacgcttctcttggaggtgagcc

SEQ ID NO: 70 - DNA, 52 bp, Homo sapiens
gcccatcccagaggcatgtggggtcaggataccagtgtttcaaggcacctgc

SEQ ID NO: 71 - DNA, 52 bp, Homo sapiens
aaatcccctagaagcctggtgtccgctatgacctccccctagggcggagcct

SEQ ID NO: 72 - DNA, 52 bp, Homo sapiens
ggtagggccgctggaccctggacacaggattggaaggaaccagcactagcag

SEQ ID NO: 73 - DNA, 52 bp, Homo sapiens
aacccctctccttgggtgcctctcccttcataggcctgagttcctggcactg

SEQ ID NO: 74 - DNA, 52 bp, Homo sapiens
cctgacatgctaacctctctgaactagcaacactggattgttcttttttaag

SEQ ID NO: 75 - DNA, 52 bp, Homo sapiens
gtggttactttctggagagagcatgtcggcatgcaggagctggagggggggt

SEQ ID NO: 76 - DNA, 1610 bp, Homo sapiens
gcgccaccgccattgccgccatcgtcgtggggcttctggggcagctagggctgcccgccgcgctgcctgcgccggaccggggcgggtccagtcccgggcgggccgtcgcgggagagaaataacatctgctttgctgccgagctcagaggagaccccagacccctcccgcagccagagggctggagcctgctcagaggtgctttgaagaggatccctgggctgcctggggaagcacccagggccagggagtgtgaccctgcaggctccacacaggactgccagaggcacacacctgctctgtctacccgagggcaccagagggcacgagaaggctggctccctggcgctgacacgtcaggcaactgaggcacaaggctggcatttctgaaccttgcccctctgcaaacacaagggggcgatggtggcactccaagcaaaggggcgtgtgggtgctgcaggaggagcacagagcactggcgcccctcccctcccgccctgcagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaagaatctaaatatttagatataa

SEQ ID NO: 77 - protein, 271 aa, Homo sapiens
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP